(19) United States
(12) Patent Application Publication
(10) Pub. No.: US 2024/0247019 A1
(43) Pub. Date: Jul. 25, 2024

(54) METHOD FOR PRODUCING BIPYRIDINE DERIVATIVE, METHOD FOR PRODUCING MACROCYCLIC COMPOUND, METHOD FOR PRODUCING METAL COMPLEX CONTAINING MACROCYCLIC COMPOUND AS LIGAND, METAL COMPLEX, ELECTRODE FOR AIR BATTERY, AND AIR BATTERY

(71) Applicant: SUMITOMO CHEMICAL COMPANY, LIMITED, Tokyo (JP)

(72) Inventors: Kentaro MASE, Osaka-shi (JP); Norifumi KOBAYASHI, Tsukuba-shi (JP); Koji ISHIWATA, Osaka-shi (JP)

(73) Assignee: SUMITOMO CHEMICAL COMPANY, LIMITED, Tokyo (JP)

(21) Appl. No.: 18/563,340

(22) PCT Filed: May 20, 2022
(86) PCT No.: PCT/JP2022/020996

§ 371 Date: Nov. 21, 2023

(30) Foreign Application Priority Data
May 24, 2021 (JP) 2021-087108

(51) Int. Cl. C07F 15/06; C07D 471/04; C07D 471/22; C07F 3/06; H01M 4/90; H01M 12/06; H01M 12/08

(52) U.S. Cl. CPC C07F 15/065; C07D 471/04; C07D 471/22; C07F 3/06; H01M 4/9008; H01M 12/06; H01M 12/08

(57) ABSTRACT

A method for producing a bipyridine derivative represented by the following formula (3) includes a step of obtaining a metal complex as an intermediate. In the formula (3), R¹³ to R²⁰ each represent a hydrogen atom or a substituent, the plurality of R¹³ to R²⁰ may be the same or different, at least one of six Rs consisting of two R¹⁴s, two R¹⁵s, and two R¹⁶s represents a substituent, at least one of two R¹⁷s represents a hydrogen atom, any two substituents of R¹³ to R²⁰ may be bonded to each other to form a ring, each of R¹⁷ to R²⁰ may contain a halogen atom or a pyrrolyl group optionally having a substituent, and at least one of R¹⁴ to R¹⁶ contains a halogen atom or a pyrrolyl group optionally having a substituent.

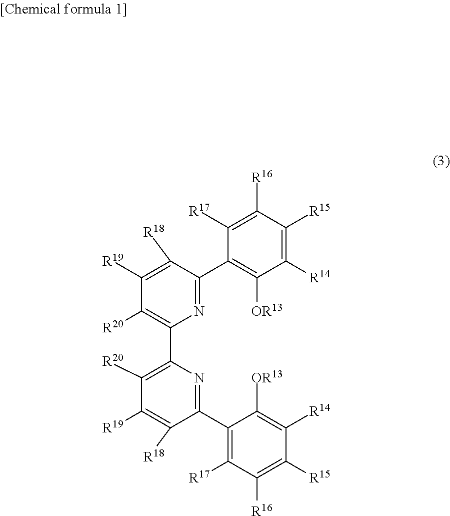

### TECHNICAL FIELD

The present invention relates to a method for producing a bipyridine derivative, a method for producing a macrocyclic compound, a method for producing a metal complex containing a macrocyclic compound as a ligand, a metal complex, an electrode for air battery, and an air battery.

### BACKGROUND ART

A bipyridine derivative has been developed as a ligand of a metal complex working as a catalyst, an electron transport material, a light-emitting material, and raw materials thereof, and use thereof is diverse.

Patent Document 1 discloses that a metal complex having a metal atom and a ligand represented by the following formula (G-5) is preferably used for a solid polymer electrolyte fuel cell, an agent for preventing deterioration of an ion-conducting film used for water electrolysis, and an antioxidant for medicines, agrochemicals, and foods, and the like. Here, the ligand represented by the following formula (G-5) is produced by the following reaction scheme.

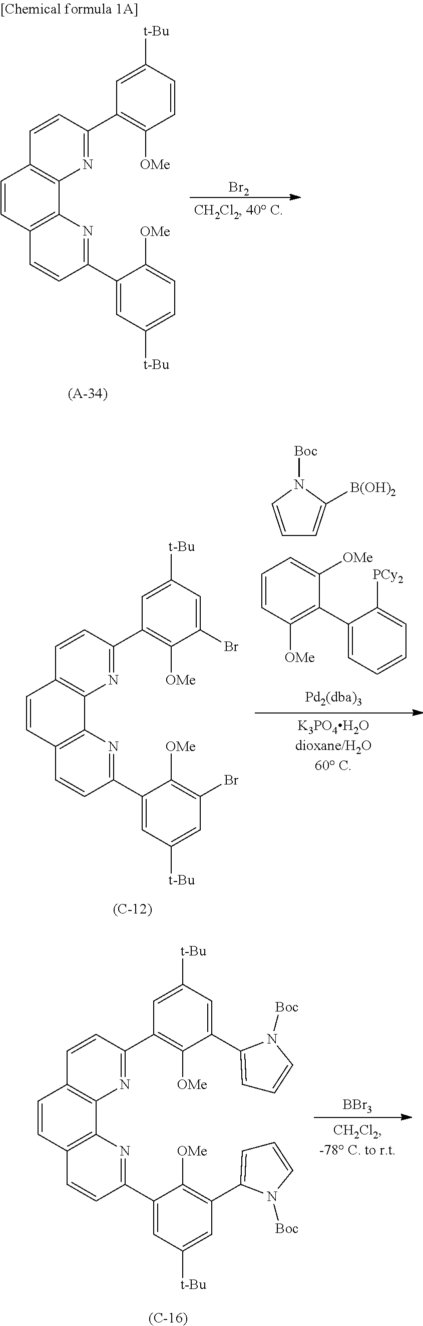

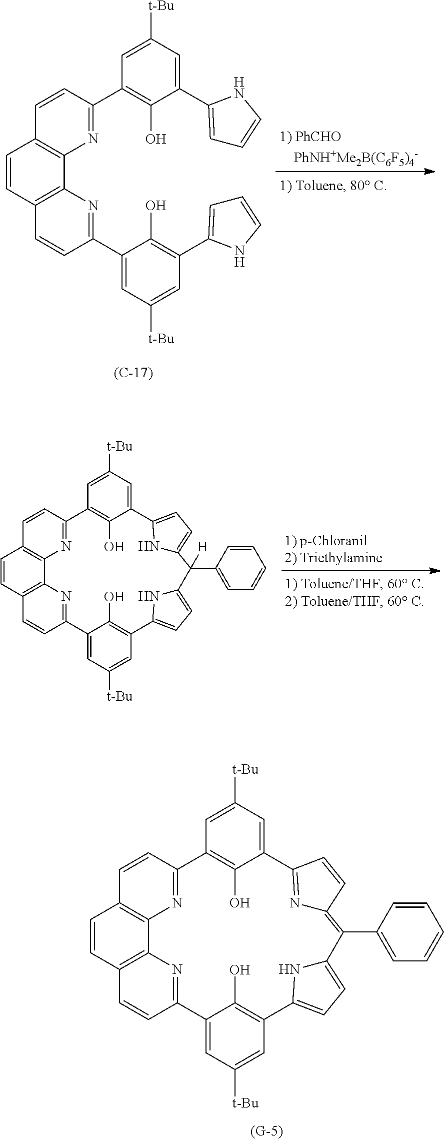

Patent Document 1 and Non-Patent Document 1 describe that the ligand represented by the formula (G-5) is obtained by brominating a compound represented by the formula (A-34) to obtain a compound represented by the formula (C-12), reacting the compound with pyrrole group to obtain a compound represented by the formula (C-16), deprotecting the compound to obtain a compound represented by the formula (C-17), and cyclizing the compound, as in the scheme indicated above.

Furthermore, Non-Patent Document 1 describes that the compound represented by the formula (A-34) is obtained by the following reaction.

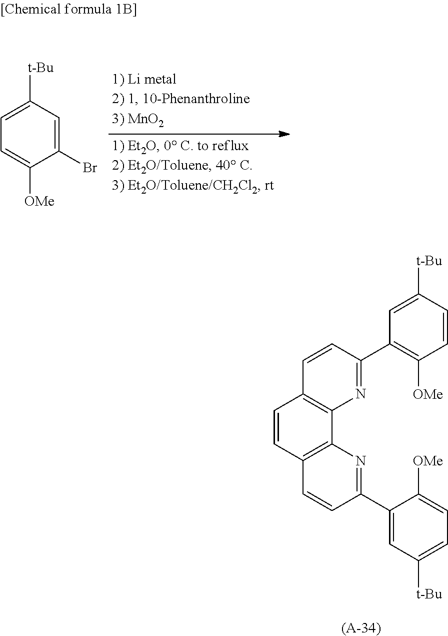 [SMILES: COc1ccc(C(C)(C)C)cc1Br ; COc1ccc(C(C)(C)C)cc1-c1ccc2ccc3ccc(-c4cc(C(C)(C)C)ccc4OC)nc3c2n1 ; "(A-34)"]

### PRIOR ART DOCUMENTS

### Patent Document

Patent Document 1: JP-B2-5422159

### Non-Patent Document

Non-Patent Document 1: Fung Lam, MaoQi Feng, Kin Shing Chan Synthesis of Dinucleating Phenanthroline-Based Ligands, Tetrahedron, 55, (1999), 8377-8384

### SUMMARY OF THE INVENTION

### Problems to be Solved by the Invention

In the above reaction scheme, the compound represented by the formula (A-34), the compound represented by the formula (C-12), the compound represented by the formula (C-16), and the compound represented by the formula (C-17) have low crystallinity, and purification thereof by crystallization is difficult even when conditions are optimized. For this reason, purification by column chromatography is required to obtain the high-purity compound and the like, a process is complicated, cost thereof is high, and it is difficult to apply the process to mass production to such an extent that the process can be used industrially. Furthermore, in the above reaction scheme, there is also a problem that a yield of the ligand represented by the formula (G-5) is low.

The present invention has been made in view of the above circumstances, and an object of the present invention is to provide a method for producing a bipyridine derivative, capable of obtaining a high-purity target product (including an intermediate) without purification by column chromatography, having a high yield, and being industrially advantageous, a method for producing a macrocyclic compound using the bipyridine derivative as a raw material, a method for producing a metal complex containing a macrocyclic compound using the macrocyclic compound as a raw material as a ligand, and a metal complex used in the method for producing a bipyridine derivative. Another object of the present invention is to provide an air electricity electrode containing the metal complex, and an air battery.

### Means for Solving the Problems

As a result of intensive studies to solve the above problems, the present inventors have focused on a fact that an intermediate of a bipyridine derivative is a multidentate ligand, and have found an industrially advantageous method for producing a bipyridine derivative via a metal complex formed by adding a metal salt to the multidentate ligand as an intermediate, thereby completing the present invention.

The present invention is the following [1] to [11].
[1] A method for producing a bipyridine derivative, including: a first step of obtaining a metal complex 1 represented by the following formula (2) from a compound represented by the following formula (1); and a second step of obtaining a bipyridine derivative represented by the following formula (3) from the metal complex 1, in which the second step includes one or both step of a halogenation reaction and a pyrrole group introducing reaction on the metal complex 1 to obtain a metal complex 2, and a demetalation step of the metal complex 2, and the number of halogen atoms contained in the bipyridine derivative is larger than the number of halogen atoms contained in the compound, or the number of pyrrolyl groups optionally having a substituent, contained in the bipyridine derivative is larger than the number of pyrrolyl groups optionally having a substituent, contained in the compound.

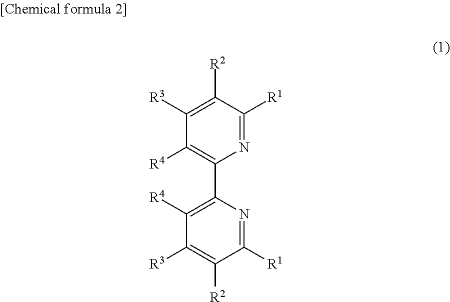

(In the formula (1), R¹ to R⁴ each independently represent a hydrogen atom or a substituent, R¹ to R⁴ may be the same or different, two R¹s may be the same or different, two R²s may be the same or different, two R¹s may be the same or different, two R⁴s may be the same or different, any two substituents of R¹ to R⁴ may be bonded to each other to form a ring, and each of R¹ to R⁴ may contain a halogen atom or a pyrrolyl group optionally having a substituent.)

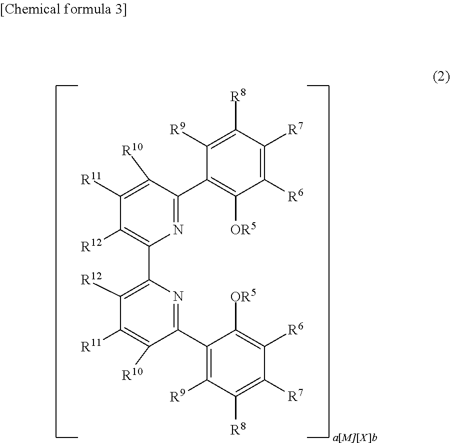

(In the formula (2), R⁵ to R¹² each independently represent a hydrogen atom or a substituent, R⁵ to R¹² may be the same or different, two R¹s may be the same or different, two R⁶s may be the same or different, two R¹s may be the same or different, two R⁸s may be the same or different, two R¹s may be the same or different, two R¹⁰s may be the same or different, two R¹¹s may be the same or different, two R¹²s may be the same or different, at least one of six Rs consisting of two R⁶s, two R⁷s, and two R⁸s represents a substituent, at least one of two R⁹s represents a hydrogen atom, any two substituents of R⁵ to R¹² may be bonded to each other to form a ring, R⁶ to R¹² may each contain a halogen atom or a pyrrolyl group optionally having a substituent, M represents any metal belonging to Groups 4 to 12 in fourth period of periodic table, X represents an anion species, a represents an integer of 1 to 3, and b represents 0 or more.)

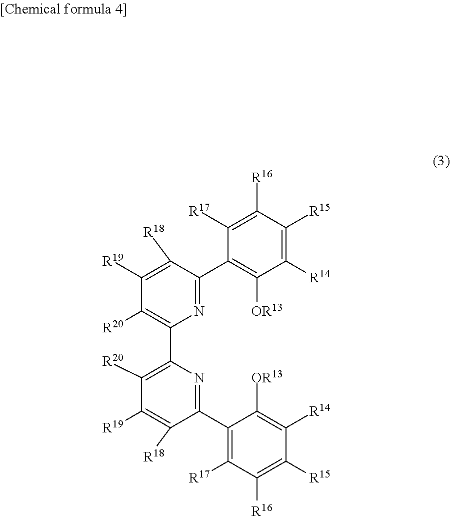

(In the formula (3), R¹³ to R²⁰ each independently represent a hydrogen atom or a substituent, R¹³ to R²⁰ may be the same or different, two R¹³s may be the same or different, two R¹⁴s may be the same or different, two R¹⁵s may be the same or different, two R¹⁶s may be the same or different, two R¹⁷s may be the same or different, two R¹⁸s may be the same or different, two R¹⁹s may be the same or different, two R²⁰s may be the same or different, at least one of six Rs consisting of two R¹⁴s, two R¹⁵s, and two R¹⁶s, at least one of two R¹⁷s represents a hydrogen atom, any two substituents of R¹³ to R²⁰ may be bonded to each other to form a ring, each of R¹⁷ to R²⁰ may contain a halogen atom or a pyrrolyl group optionally having a substituent, and at least one of R¹⁴ to R¹⁶ contains a halogen atom or a pyrrolyl group optionally having a substituent.)
[2] The method for producing a bipyridine derivative according to [1], in which the demetalation step is performed by using an amine represented by the following formula (4) to react.

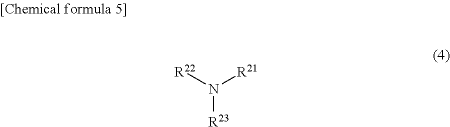

(In the formula (4), R²¹ to R²³ each independently represent a hydrogen atom or a substituent.)
[3] The method for producing a bipyridine derivative according to [1] or [2], in which the first step includes a step for reacting the compound and a metal represented by the M and an anion species represented by the X.
[4] The method for producing a bipyridine derivative according to any one of [1] to [3], in which the second step includes a deprotection step after the demetalation step.
[5] The method for producing a bipyridine derivative according to any one of [1] to [4], including a step of isolating the metal complex 1, the metal complex 2, or the bipyridine derivative by crystallization.
[6] A method for producing a macrocyclic compound, including ring-closing reaction of the bipyridine derivative having two or more pyrrolyl groups optionally having a substituent, which has been produced by the method for producing a bipyridine derivative according to any one of [1] to [5] to obtain a macrocyclic compound represented by the following formula (5).

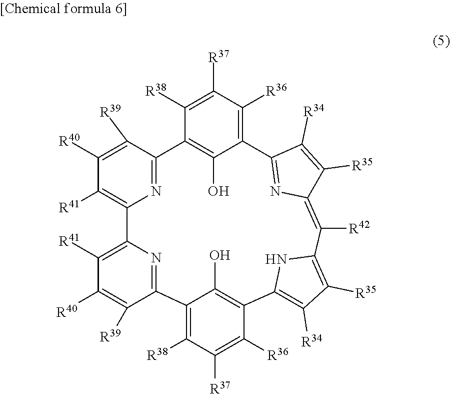

(In the formula (5), R³⁴ to R⁴² each independently represent a hydrogen atom or a substituent, R³⁴ to R⁴² may be the same or different, two R³⁴s may be the same or different, two R³⁵s may be the same or different, two R³⁶s may be the same or different, two R³⁷s may be the same or different, two R³⁸s may be the same or different, two R³⁹s may be the same or different, two R⁴⁰s may be the same or different, two R⁴¹s may be the same or different, and any two substituents of R³⁴ to R⁴² may be bonded to each other to form a ring.)
[7] A method for producing a metal complex containing a macrocyclic compound as a ligand, the method comprising the reaction of the macrocyclic compound produced by the method for producing a macrocyclic compound according to [6] as a ligand with a metal salt containing a metal belonging to fourth to sixth periods of periodic table.
[8] A metal complex represented by the following formula (6).

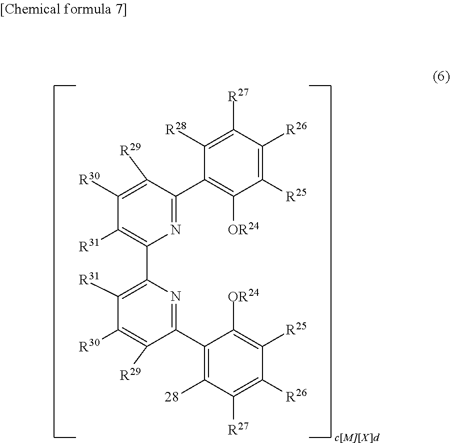

(In the formula (6), R²⁴ represents a substituent, R²⁵ to R³¹ each independently represent a hydrogen atom or a substituent, R²⁴ to R³¹ may be the same or different, two R²⁴s may be the same or different, two R²⁵s may be the same or different, two R²⁶s may be the same or different, two R²⁷s may be the same or different, two R²⁸s may be the same or different, two R³⁰s may be the same or different, two R³¹s may be the same or different, at least one of six Rs consisting of two R²⁵s, two R²⁶s, and two R²⁷s represents a substituent, at least one of two R²⁸s represents a hydrogen atom, any two substituents of R²⁴ to R³¹ may be bonded to each other to form a ring, M represents any metal belonging to Groups 4 to 12 in fourth period of periodic table, X represents an anion species, c represents an integer of 1 to 3, and d represents 0 or more.)
[9] An electrode for air battery, including a catalyst layer containing an electrode catalyst containing the metal complex according to [8], a conductive material, and a binder.
[10] An air battery including: the electrode for air battery according to [9]; and an anode, in which the anode contains a anode active material, and the anode active material contains one or more selected from the group consisting of zinc, iron, aluminum, magnesium, lithium, hydrogen, and ions thereof.
[11] The air battery according to [10], in which the anode active material contains one or more selected from the group consisting of magnesium and magnesium ions.

### Effect of the Invention

According to the present invention, it is possible to provide a method for producing a bipyridine derivative, capable of obtaining a high-purity target product (including intermediates) without purification by column chromatography, having a high yield, and being industrially advantageous, a method for producing a macrocyclic compound using the bipyridine derivative as a raw material, a method for producing a metal complex containing a macrocyclic compound using the macrocyclic compound as a raw material as a ligand, and a metal complex used in the method for producing a bipyridine derivative. Furthermore, it is possible to provide an air electricity electrode containing the metal complex and an air battery.

### BRIEF DESCRIPTION OF THE DRAWINGS

FIG. 1 is a schematic configuration diagram illustrating an example of an air battery of the present embodiment.

### MODE FOR CARRYING OUT THE INVENTION

### <<Method for Producing Bipyridine Derivative>>

A method for producing a bipyridine derivative of the present embodiment includes a first step of obtaining a metal complex 1 represented by the following formula (2) from a compound represented by the following formula (1), and a second step of obtaining a bipyridine derivative represented by the following formula (3) from the metal complex 1. The second step includes one or both step of a halogenation reaction and a pyrrole group introducing reaction on the metal complex 1 to obtain a metal complex 2, and a demetalation step of demetallizing a metal from the metal complex 2.

The number of halogen atoms contained in the bipyridine derivative is larger than the number of halogen atoms contained in the compound, or the number of pyrrolyl groups optionally having a substituent, contained in the bipyridine derivative is larger than the number of pyrrolyl groups optionally having a substituent, contained in the compound.

Hereinafter, the compound represented by the following formula (1), the metal complex 1 represented by the following formula (2), the metal complex 2, and the bipyridine derivative represented by the following formula (3) in the present embodiment will be described. In addition, conditions of the first step and the second step will be described.

Note that the compound or the metal complex represented by any one of the formulas (1) to (3) is not included in a macrocyclic compound described later. Definition of the macrocyclic compound will be described later.

### <Compound Represented by Formula (1)>

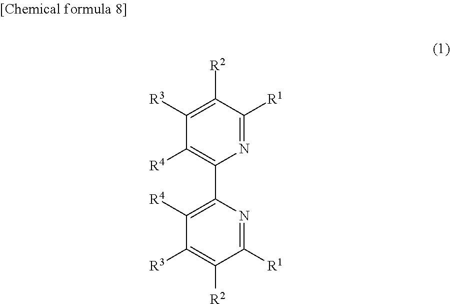

In the formula (1), R¹ to R⁴ each independently represent a hydrogen atom or a substituent, R¹ to R⁴ may be the same or different, two R¹s may be the same or different, two Res may be the same or different, two R¹s may be the same or different, two R⁴s may be the same or different, any two substituents of R¹ to R⁴ may be bonded to each other to form a ring, and each of R¹ to R⁴ may contain a halogen atom or a pyrrolyl group optionally having a substituent.

When each of R¹ to R⁴ is a substituent, the substituent is a hydrocarbyl group or a monovalent group having a hetero element (an element other than carbon and hydrogen), and a hydrocarbyl group is preferable. Examples of the hydrocarbyl group include an alkyl group, an aryl group, and an aralkyl group, and an alkyl group and an aryl group are preferable. The monovalent group having a hetero atom is preferably, for example, a halogen atom, a pyrrolyl group, a hydroxy group, a carbonyl group, a carboxyl group, a carbamoyl group, an amino group, a sulfonic acid group, a nitro group, a phosphonic acid group, a boronic acid group, a boronate group, a silyl group, an alkoxy group, a heteroaryl group, an aryloxy group, an aralkyloxy group, or a silyloxy group.

Each of these substituents may further have a substituent, or does not have to further have a substituent. Hereinafter, a substituent included in a substituent as R¹ to R⁴ and the like is referred to as a “substituent (1)” in order to be distinguished from the substituent as R¹ to R⁴ and the like. In the present specification, inclusion of the substituent (1) in a substituent means that one or more hydrogen atoms in the substituent are replaced with groups (substituents (1)) other than a hydrogen atom. Examples of the substituent (1) include an alkyl group, an aryl group, an aralkyl group, a halogen atom, a pyrrolyl group, a hydroxy group, a carbonyl group, a carboxyl group, a carbamoyl group, an amino group, a sulfonic acid group, a nitro group, a phosphonic acid group, a boronic acid group, a boronate group, a silyl group, and an alkoxy group, and an alkyl group, an aryl group, an aralkyl group, a halogen atom, a pyrrolyl group, a hydroxy group, a carbonyl group, a carboxyl group, an amino group, a nitro group, a boronic acid group, a boronate group, and an alkoxy group are preferable. Specific examples and preferable forms of these substituents include those equivalents to substituents exemplified for the substituent as R¹ to R⁴ and the like described later. Hereinafter, when the number of carbon atoms is defined in R¹ to R⁴ and the like, the number of carbon atoms contained in the substituent (1) is included.

Examples of the alkyl group in the substituent as R¹ to R⁴ and the like include a methyl group, an ethyl group, a propyl group, an isopropyl group, a butyl group, an isobutyl group, a tert-butyl group, a pentyl group, a hexyl group, a norbonyl group, a nonyl group, a decyl group, a 3,7-dimethyloctyl group, a dodecyl group, a pentadecyl group, an octadecyl group, and a docosyl group, and a methyl group and a tert-butyl group are preferable. The alkyl group may have the substituent (1), or does not have to have the substituent (1). The number of carbon atoms of the alkyl group is not particularly limited, but is preferably 1 or more and 20 or less, and more preferably 1 or more and 8 or less from a viewpoint of availability and cost.

Examples of the aryl group in the substituent as R¹ to R⁴ and the like include a phenyl group, a biphenyl group, a terphenyl group, a naphthyl group, a phenanthryl group, an anthracenyl group, a benzophenanthryl group, a benzanthracenyl group, a chrysenyl group, a pyrenyl group, a fluoranthenyl group, a triphenylenyl group, a benzofluoranthenyl group, a dibenzoanthracenyl group, a perylenyl group, and a helisenyl group, and a phenyl group is preferable. The aryl group may have the substituent (1), or does not have to have the substituent (1). The number of carbon atoms of the aryl group is not particularly limited, but is preferably 6 or more and 40 or less, and more preferably 6 or more and 20 or less.

Examples of the aralkyl group in the substituent as R¹ to R⁴ and the like include a benzyl group, a naphthylmethyl group, and an anthracenylmethyl group. Examples of the aralkyl group having a substituent include a (2-methylphenyl) methyl group, a (3-methylphenyl) methyl group, a (4-methylphenyl) methyl group, a (2,3-dimethylphenyl) methyl group, a (2,4-dimethylphenyl) methyl group, a (2,5-dimethylphenyl) methyl group, a (2,6-dimethylphenyl) methyl group, a (3,4-dimethylphenyl) methyl group, a (4,6-dimethylphenyl) methyl group, a (2,3,4-trimethylphenyl) methyl group, a (2,3,5-trimethylphenyl) methyl group, a (2,3,6-trimethylphenyl) methyl group, a (3,4,5-trimethylphenyl) methyl group, a (2,4,6-trimethylphenyl) methyl group, a (2,3,4,5-tetramethylphenyl) methyl group, (2,3,4,6-tetramethylphenyl) methyl group, a (2,3,5,6-tetramethylphenyl) methyl group, a (pentamethylphenyl) methyl group, an (ethylphenyl) methyl group, a (n-propylphenyl) methyl group, an (isopropylphenyl) methyl group, a (n-butylphenyl) methyl group, a (sec-butylphenyl) methyl group, a (tert-butylphenyl) methyl group, a (n-pentylphenyl) methyl group, a (neopentylphenyl) methyl group, a (n-hexylphenyl) methyl group, a (n-octylphenyl) methyl group, a (n-decylphenyl) methyl group, and a (n-decylphenyl) methyl group. The number of carbon atoms of the aralkyl group is not particularly limited, but is preferably 7 or more and 40 or less, and more preferably 7 or more and 20 or less.

Examples of the halogen atom in the substituent as R¹ to R⁴ and the like include a fluorine atom, a chlorine atom, a bromine atom, and an iodine atom, and a chlorine atom, a bromine atom, and an iodine atom are preferable, and a bromine atom and an iodine atom are more preferable.

The pyrrolyl group in the substituent as R¹ to R⁴ and the like is a monovalent group obtained by removing one hydrogen atom from pyrrole. The pyrrolyl group in the present embodiment may be a pyrrolyl group having a substituent. The substituent contained in the pyrrolyl group is the above-described substituent (1).

Note that, in the present specification, the pyrrolyl group is not included in a heteroaryl group described later.

The silyl group in the substituent as R¹ to R⁴ and the like may have a substituent of a hydrocarbon group, and examples thereof include: a monosubstituted silyl group having 1 to 20 carbon atoms, such as a methylsilyl group, an ethylsilyl group, or a phenylsilyl group; a disubstituted silyl group having a substituent of a hydrocarbon group having 2 to 20 carbon atoms, such as a dimethylsilyl group, a diethylsilyl group, or a diphenylsilyl group; and a trisubstituted silyl group having a substituent of a hydrocarbon group having 3 to 20 carbon atoms, such as a trimethylsilyl group, a triethylsilyl group, a tri-n-propylsilyl group, a triisopropylsilyl group, a tri-n-butylsilyl group, a tri-sec-butylsilyl group, a tri-tert-butylsilyl group, a tri-isobutylsilyl group, a tert-butyl-dimethylsilyl group, a tri-n-pentylsilyl group, a tri-n-hexylsilyl group, a tricyclohexylsilyl group, or a triphenylsilyl group, and a trimethylsilyl group, a tert-butyldimethylsilyl group, and a triphenylsilyl group are preferable.

Examples of the alkoxy group in the substituent as R¹ to R⁴ and the like include a methoxy group, an ethoxy group, a n-propoxy group, an isopropoxy group, a n-butoxy group, a sec-butoxy group, a tert-butoxy group, a n-pentyloxy group, a neopentyloxy group, a n-hexyloxy group, a n-octyloxy group, a n-nonyloxy group, a n-decyloxy group, a n-dodecyloxy group, a n-undecyloxy group, a n-dodecyloxy group, a tridecyloxy group, a tetradecyloxy group, a n-pentadecyloxy group, a hexadecyloxy group, a heptadecyloxy group, an octadecyloxy group, a nonadecyloxy group, and a n-eicosyloxy group, and a methoxy group, an ethoxy group, and a tert-butoxy group are preferable. The alkoxy group may have the substituent (1), or does not have to have the substituent (1).

The heteroaryl group in the substituent as R¹ to R⁴ and the like is a group in which a carbon atom constituting a ring of an aryl group is replaced with a heteroatom or a carbonyl group. The heteroaryl group having 4 to 36 carbon atoms contains: a monocyclic heteroaryl group; a fused ring heteroaryl group; a monovalent group in which two or more monocyclic rings and/or fused ring heteroaryl groups are directly bonded to each other or indirectly bonded to each other via a hetero atom (an oxygen atom, a nitrogen atom, a sulfur atom, or the like) or a carbonyl group (—CO—); and a monovalent group in which one or more monocyclic rings and/or fused ring heteroaryl groups and one or more monocyclic rings and/or fused ring aryl groups are directly bonded to each other or indirectly bonded to each other via a hetero atom (an oxygen atom, a nitrogen atom, a sulfur atom, or the like) or a carbonyl group (—CO—).

A remaining bond of a nitrogen atom that indirectly bonds heteroaryl groups to each other is bonded to, for example, an alkyl group optionally having the substituent (1) or an aryl group optionally having the substituent (1). Note that a fused ring contained in a heteroaryl group of the fused ring may be a fused ring of two or more heterocyclic rings or a fused ring of one or more heterocyclic rings and one or more aromatic rings.

Specific examples of the heteroaryl group include a group in which one hydrogen atom is removed from each of pyridine, pyrazine, pyrimidine, furan, thiophene, thiazole, imidazole, oxazole, benzofuran, benzothiophene, isoquinoline, and quinazoline, and pyridine, pyrazine, pyrimidine, furan, and thiophene are preferable, and pyridine, furan, and thiophene are more preferable.

Examples of the aryloxy group in the substituent as R¹ to R⁴ and the like include a phenoxy group, a naphthoxy group, and an anthracenoxy group. Examples of the aryloxy group having a substituent include a 2-methylphenoxy group, a 3-methylphenoxy group, a 4-methylphenoxy group, a 2,3-dimethylphenoxy group, a 2,4-dimethylphenoxy group, a 2,5-dimethylphenoxy group, a 2,6-dimethylphenoxy group, a 3,4-dimethylphenoxy group, a 3,5-dimethylphenoxy group, a 2,3,4-trimethylphenoxy group, a 2,3,5-trimethylphenoxy group, a 2,3,6-trimethylphenoxy group, a 2,4,5-trimethylphenoxy group, a 2,4,6-trimethylphenoxy group, a 3,4,5-trimethylphenoxy group, a 2,3,4,5-tetramethylphenoxy group, a 2,3,4,6-tetramethylphenoxy group, a 2,3,5,6-tetramethylphenoxy group, a pentamethylphenoxy group, an ethylphenoxy group, a n-propylphenoxy group, an isopropylphenoxy group, a n-butylphenoxy group, a sec-butylphenoxy group, a tert-butylphenoxy group, a n-hexylphenoxy group, a n-octylphenoxy group, a n-decylphenoxy group, and a n-tetradecylphenoxy group. The number of carbon atoms of the aryloxy group is not particularly limited, but is preferably 6 or more and 40 or less, and more preferably 6 or more and 20 or less.

Examples of the aralkyloxy group in the substituent as R¹ to R⁴ and the like include a benzyloxy group, a naphthylmethoxy group, and an anthracenylmethoxy group. Examples of the aralkyloxy group having a substituent include a (2-methylphenyl) methoxy group, a (3-methylphenyl) methoxy group, a (4-methylphenyl) methoxy group, a (2,3-dimethylphenyl) methoxy group, a (2,4-dimethylphenyl) methoxy group, a (2,5-dimethylphenyl) methoxy group, a (2,6-dimethylphenyl) methoxy group, a (3,4-dimethylphenyl) methoxy group, a (3,5-dimethylphenyl) methoxy group, a (2,3,4-trimethylphenyl) methoxy group, a (2,3,5-trimethylphenyl) methoxy group, a (2,3,6-trimethylphenyl) methoxy group, a (2,4,5-trimethylphenyl) methoxy group, a (2,4,6-trimethylphenyl) methoxy group, a (3,4,5-trimethylphenyl) methoxy group, a (2,3,4,5-tetramethylphenyl) methoxy group, a (2,3,4,6-tetramethylphenyl) methoxy group, a (2,3,5,6-tetramethylphenyl) methoxy group, a (pentamethylphenyl) methoxy group, an (ethylphenyl) methoxy group, a (n-propylphenyl) methoxy group, an (isopropylphenyl) methoxy group, a (n-butylphenyl) methoxy group, a (sec-butylphenyl) methoxy group, a (tert-butylphenyl) methoxy group, a (n-hexylphenyl) methoxy group, a (n-octylphenyl) methoxy group, and a (n-decylphenyl) methoxy group. Among these groups, a benzyloxy group is preferable. The number of carbon atoms of the aralkyloxy group is not particularly limited, but is preferably 7 or more and 40 or less, and more preferably 7 or more and 20 or less.

The silyloxy group in the substituent as R¹ to R⁴ and the like may have a substituent of a hydrocarbon group, and examples thereof include a trimethylsilyloxy group, a triethylsilyloxy group, a tri-n-butylsilyloxy group, a triphenylsilyloxy group, a triisopropylsilyloxy group, a tert-butyldimethylsilyloxy group, a dimethylphenylsilyloxy group, and a methyldiphenylsilyloxy group, and a trimethylsilyloxy group, a triphenylsilyloxy group, and a triisopropylsilyloxy group are preferable.

The amino group in the substituent as R¹ to R⁴ and the like may have a substituent of a hydrocarbon group, and examples thereof include a dimethylamino group, a diethylamino group, a di-n-propylamino group, a diisopropylamino group, a di-n-butylamino group, a di-sec-butylamino group, a di-tert-butylamino group, a di-isobutylamino group, a tert-butylisopropylamino group, a di-n-hexylamino group, a di-n-octylamino group, a di-n-decylamino group, a diphenylamino group, a bistrimethylsilylamino group, a bis-tert-butyldimethylsilylamino group, a pyrrolidinyl group, a piperidinyl group, a carbazolyl group, a dihydroindolyl group, and a dihydroisoindolyl group.

Examples of the carbonyl group in the substituent as R¹ to R⁴ and the like include a methoxycarbonyl group, a tert-butoxycarbonyl group, an ethoxycarbonyl group, and an aldehyde group.

Examples of the boronate group in the substituent as R¹ to R⁴ and the like include a boronic acid pinacol ester group, a boronic acid-1,3-propanediol ester group, a boronic acid catechol ester group, and a boronic acid dimethyl ester group.

Among the above-described structures, R¹ is preferably a phenyl group having (—OR⁵), (—R⁶), (—R⁷), (—R⁸), and (—R⁹) in the following formula (2) as substituents.

Two R¹s may be the same or different, but are preferably the same.

Two R²s may be the same or different, but are preferably the same.

Two R¹s may be the same or different, but are preferably the same.

Two R⁴s may be the same or different, but are preferably the same.

R² is preferably a hydrogen atom or an alkyl group having 1 or more and 20 or less carbon atoms, and more preferably a hydrogen atom.

R³ is preferably a hydrogen atom or an alkyl group having 1 or more and 20 or less carbon atoms, and more preferably a hydrogen atom.

R⁴ is preferably a hydrogen atom or an alkyl group having 1 or more and 20 or less carbon atoms.

When R¹ to R⁴ have a halogen atom, the number of halogen atoms contained in the compound represented by the formula (1) is preferably 1 to 4, and more preferably 1 or 2. A halogen atom is preferably contained in R¹ as the substituent (1) when R¹ is a substituent. R¹ to R⁴ do not have to have a halogen atom.

When R¹ to R⁴ have a pyrrolyl group optionally having a substituent, the number of pyrrolyl groups optionally having a substituent contained in the compound represented by the formula (1) is preferably 1 to 4, and more preferably 1 or 2. The pyrrolyl group optionally having a substituent is preferably contained in R¹ as the substituent (1) when R¹ is a substituent. R¹ to R⁴ do not have to have a pyrrolyl group optionally having a substituent.

Any two substituents of the R¹ to R⁴ may be bonded to each other to form a ring.

In particular, two R⁴s are preferably bonded to each other to form a ring, and two R⁴s are preferably bonded to each other to form a ring and are fused, whereby the compound represented by the formula (1) is a phenanthroline derivative represented by the following formula (7).

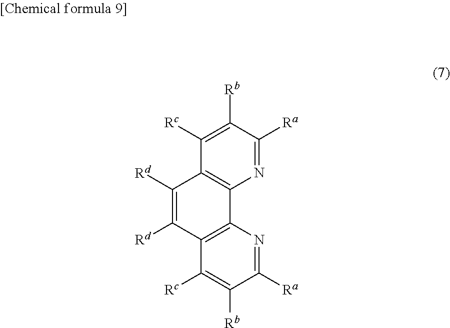

R^{a} to R^{d} each independently represent a hydrogen atom or a substituent, any two substituents of R^{a} to R^{c} are preferably not bonded to each other to form a ring, and two substituents of the R^{d} may be bonded to each other to form a ring. Examples of the substituent when the two substituents of R^{a} to R^{d} are not bonded to each other to form a ring are the same as those of the substituents of R¹ to R⁴.

Examples of the compound represented by the formula (1) include compounds represented by following formulas (A-1) to (A-43). Among these compounds, the compounds in which each of two R¹s is a phenyl group having a substituent, represented by (A-28) to (A-42) are preferable, and the compounds represented by (A-34) to (A-37) and (A-39) to (A-42), represented by the formula (7) are more preferable. Hereinafter, in chemical formulas described in the present specification, “Me” means a methyl group, “t-Bu” means a tert-butyl group, “Boc” means a tert-butoxycarbonyl group, “Bn” means a benzyl group, “dba” means dibenzylideneacetone, and “Cy” means a cyclohexyl group.

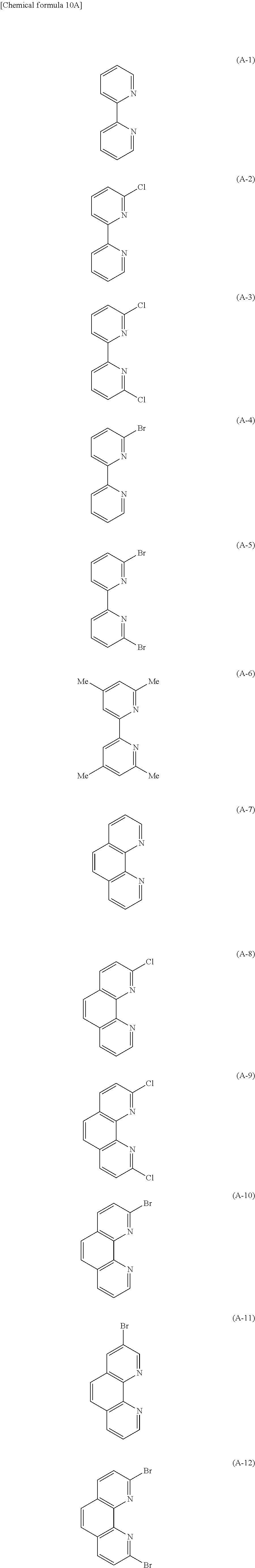

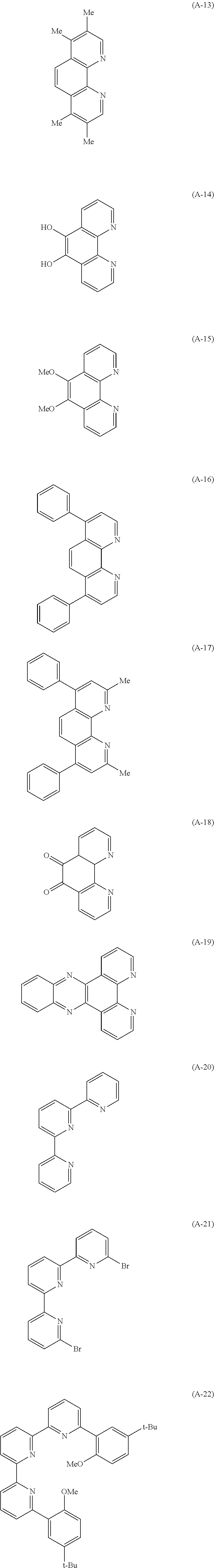

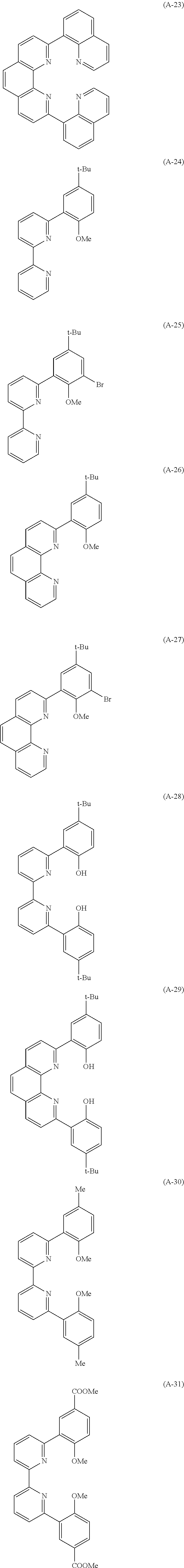

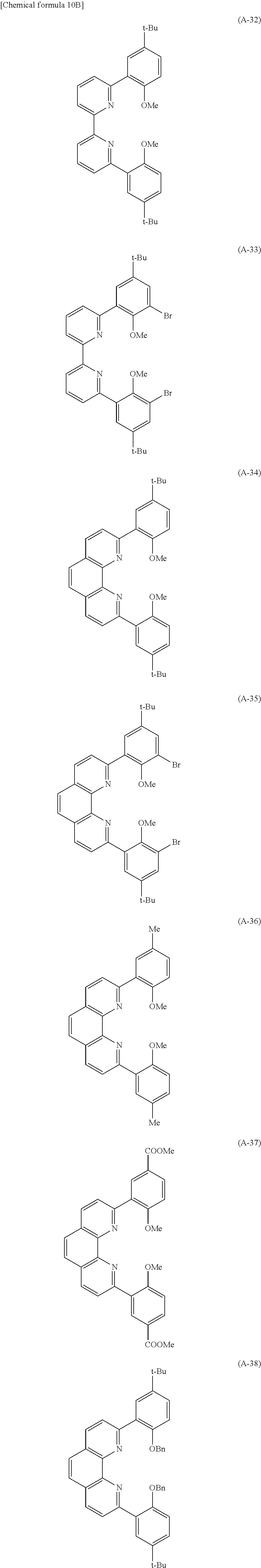

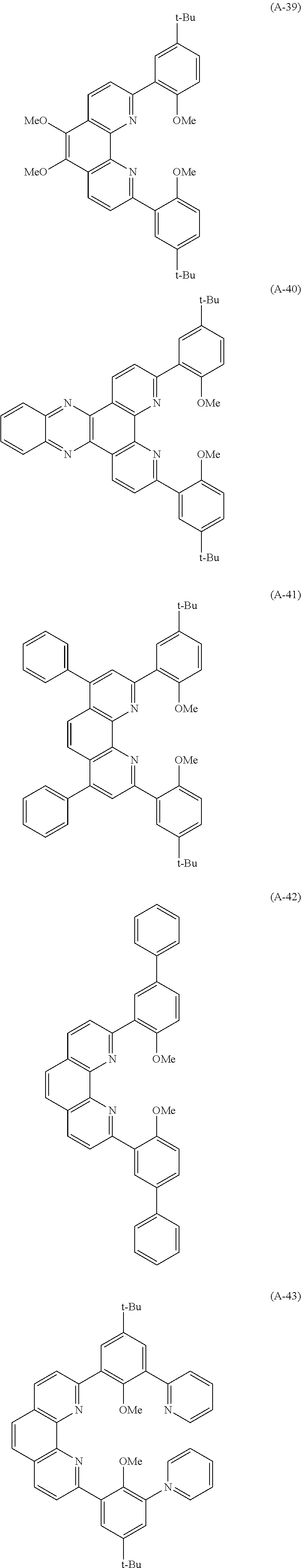

### <Metal Complex 1 Represented by Formula (2)>

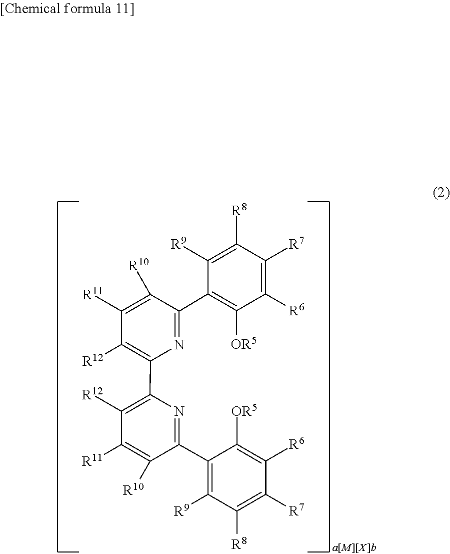

In the formula (2), R⁵ to R¹² each independently represent a hydrogen atom or a substituent, R⁵ to R¹² may be the same or different, two R⁵s may be the same or different, two R⁶s may be the same or different, two R⁷s may be the same or different, two R⁸s may be the same or different, two R⁹s may be the same or different, two R¹⁰s may be the same or different, two R¹¹s may be the same or different, two R¹²s may be the same or different, at least one of six Rs consisting of two R⁶s, two R⁷s, and two R⁸s represents a substituent, at least one of two R⁹s represents a hydrogen atom, any two substituents of R⁵ to R¹² may be bonded to each other to form a ring, R⁶ to R¹² may each contain a halogen atom or a pyrrolyl group optionally having a substituent, M represents any metal belonging to Groups 4 to 12 in the fourth period of the periodic table, X represents an anion species, a represents an integer of 1 to 3, and b represents 0 or more.

In the metal complex 1 represented by the formula (2), when R⁵ is a substituent, R⁵ is preferably a substituent capable of converting an —OR⁵ site into an —OH structure by converting the substituent into hydrogen, that is, a protecting group. Specific examples of the protecting group include a methyl group, an isopropyl group, a cyclohexyl group, a tert-butyl group, a benzyl group, a methoxymethyl group, a benzyloxymethyl group, a methoxyethoxymethyl group, a trimethylsilyl group, a tert-butyldimethylsilyl group, a triisopropylsilyl group, a methylcarbonyl group, a phenylcarbonyl group, and a tert-butoxycarbonyl group. Among these groups, a methyl group, a benzyl group, a methoxymethyl group, a trimethylsilyl group, a tert-butyldimethylsilyl group, and a tert-butoxycarbonyl group are preferable, and a methyl group is more preferable.

R⁶ to R⁸ each independently represent a hydrogen atom or a substituent, and examples of the substituent include those equivalents to the substituents exemplified as R¹ to R⁴ in the compound represented by the formula (1).

R⁶ is preferably a hydrogen atom, a halogen atom, an alkyl group having 1 or more and 20 or less carbon atoms, a heteroaryl group having 4 to 36 carbon atoms, or a pyrrolyl group optionally having a substituent, and more preferably a hydrogen atom, a bromine atom, or a pyrrolyl group optionally having a substituent.

R⁷ is preferably a hydrogen atom, a halogen atom, or an alkyl group having 1 or more and 20 or less carbon atoms, and more preferably a hydrogen atom.

R⁸ is preferably a substituent rather than a hydrogen atom.

R⁸ is preferably a hydrogen atom, a halogen atom, an alkyl group having 1 or more and 20 or less carbon atoms, a heteroaryl group having 4 to 36 carbon atoms, or a pyrrolyl group optionally having a substituent, and more preferably a tert-butyl group.

At least one of six Rs consisting of two R⁶s, two R⁷s, and two R⁸s is a substituent. Preferably, two to four of R⁶ to R⁸ are substituents. More preferably, two or four of R⁶ to R⁸ are substituents.

R⁹ represents a hydrogen atom or a substituent, and examples thereof include those equivalents to those described above as R³ in the compound represented by the formula (1).

At least one of two R⁹s is a hydrogen atom. Each of two R⁹s is preferably a hydrogen atom.

R¹⁰, R¹¹, and R¹² each independently represent a hydrogen atom or a substituent, and examples thereof include those equivalents to R², R³, and R⁴ in the compound represented by the formula (1), and preferable examples thereof are also the same.

The plurality of R⁵ to R¹² may be each independently the same or different. Preferably, two R⁵s are the same, two R⁶s are the same, two R⁷s are the same, two R⁸s are the same, two R⁹s are the same, two R¹⁰s are the same, two R¹¹s are the same, and two R¹²s are the same.

Any two of R⁵ to R¹² may be bonded to each other to form a ring.

Similarly to R⁴ described above, two R¹²s are preferably bonded to each other to form a ring, and two R¹²s are preferably bonded to each other to form a ring, whereby the metal complex 1 represented by the formula (2) is a phenanthroline derivative.

Each of R⁶ to R¹² may contain a halogen atom or a pyrrolyl group optionally having a substituent. That is, each of R⁶ to R¹² may be a halogen atom or a pyrrolyl group optionally having a substituent, and when each of R⁶ to R¹² is a substituent, the substituent (1) may be a halogen atom or a pyrrolyl group optionally having a substituent.

When R⁶ to R¹² have a halogen atom, the number of halogen atoms contained in the metal complex 1 represented by the formula (2) is preferably 1 to 4, and more preferably 1 or 2. When the metal complex 1 contains a halogen atom, R⁶ or R⁸ is preferably a halogen atom as described above. A halogen atom may be contained in R⁶ or R⁸ as the substituent (1) when R⁶ or R⁸ is a substituent. R⁶ to R¹² do not have to have a halogen atom.

When R⁶ to R¹² have a pyrrolyl group optionally having a substituent, the number of pyrrolyl groups optionally having a substituent contained in the metal complex 1 represented by the formula (2) is preferably 1 to 4, and more preferably 1 or 2. When the metal complex 1 contains a pyrrolyl group optionally having a substituent, R⁶ or R⁸ is preferably a pyrrolyl group optionally having a substituent. A pyrrolyl group optionally having a substituent may be contained in R⁶ or R⁸ as the substituent (1) when R⁶ or R⁸ is a substituent. R⁶ to R¹² do not have to have a pyrrolyl group optionally having a substituent.

a represents an integer of 1 to 3. That is, a is 1, 2, or 3, and preferably 1.

M is any metal belonging to Groups 4 to 12 in the fourth period of the periodic table.

Examples of M include aluminum, titanium, vanadium, chromium, manganese, iron, cobalt, nickel, copper, and zinc. Among these metals, cobalt, nickel, copper, and zinc capable of forming a water-soluble complex ion with a water-soluble amine exemplified as a preferable example in a water-soluble amine represented by the following formula (4) are preferable, and copper and zinc are more preferable.

M preferably has a positive charge, and the valence of the positive charge is more preferably 1 to 4, still more preferably 1 or 2, and particularly preferably 2.

The metal complex 1 represented by the formula (2) is preferably electrically neutral as a whole.

X represents an anion species, and examples thereof include an anion that electrically neutralizes the positive charge of M. Specific examples thereof include: an inorganic acid ion such as a fluoride ion, a chloride ion, a bromide ion, an iodide ion, a sulfide ion, an oxide ion, a hydroxide ion, a hydride ion, a sulfite ion, a phosphate ion, a hexafluorophosphate ion, a carbonate ion, a sulfate ion, a nitrate ion, a perchlorate ion, or a bicarbonate ion; and an organic acid ion such as an acetate ion, a 2-ethylhexanoate ion, a trifluoroacetate ion, a thiocyanate ion, a methanesulfonate ion, a trifluoromethanesulfonate ion, an acetylacetonate, a tetrafluoroborate ion, a tetraphenylborate ion, or a stearate ion. Among these ions, a chloride ion, a bromide ion, and an iodide ion are preferable.

b represents the number of Xs in the metal complex, represents a number of 0 or more, may be an integer or a decimal, and is determined such that the valence of a partial complex ion of the metal complex excluding [X]_{b} is equal to a number obtained by multiplying the valence of X by b. b is usually a number of 0 to 3, and preferably 2.

A partial structure surrounded by [ ]ₐ in the metal complex 1 represented by the formula (2) may be negatively charged due to elimination of a proton, and is preferably neutral.

b Xs may be formed of a plurality of species, and a combination of the plurality of species is preferably selected from the group consisting of a fluoride ion, a chloride ion, a bromide ion, an iodide ion, an acetate ion, a trifluoromethanesulfonate ion, a tetrafluoroborate ion, and a perchlorate ion, and more preferably selected from the group consisting of a chloride ion and a bromide ion.

Examples of the metal complex 1 represented by the formula (2) include metal complexes represented by following formulas (B-1) to (B-32). Among these metal complexes, examples in which the metal complex 1 represented by the formula (2) has a halogen atom are the metal complexes 1 represented by formulas (B-2) and (B-23) to (B-25), and examples in which the metal complex 1 represented by the formula (2) has a pyrrolyl group optionally having a substituent are the metal complexes represented by formulas (B-26) to (B-31). Among these metal complexes, formulas (B-1) to (B-13), (B-17), and (B-22) to (B-32) in which M is zinc are preferable.

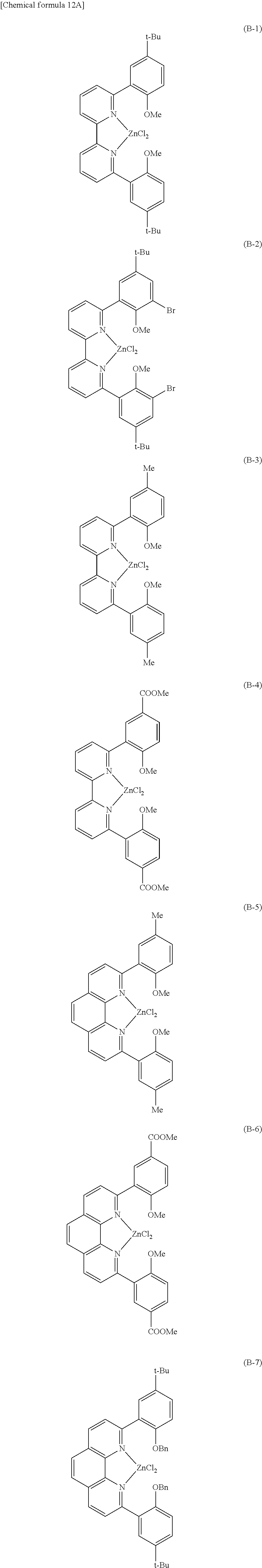

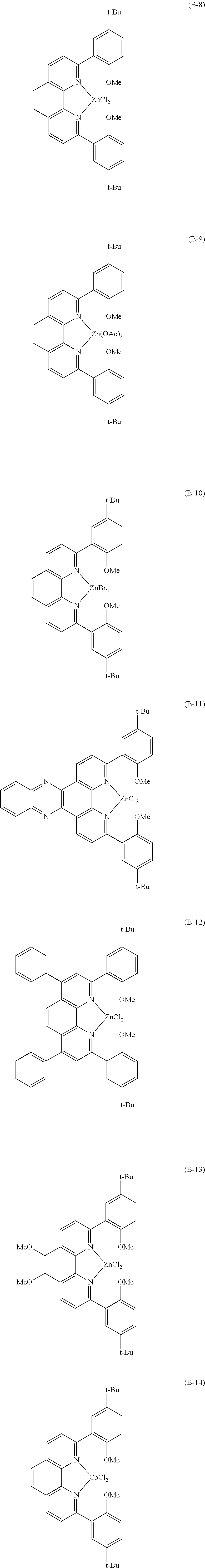

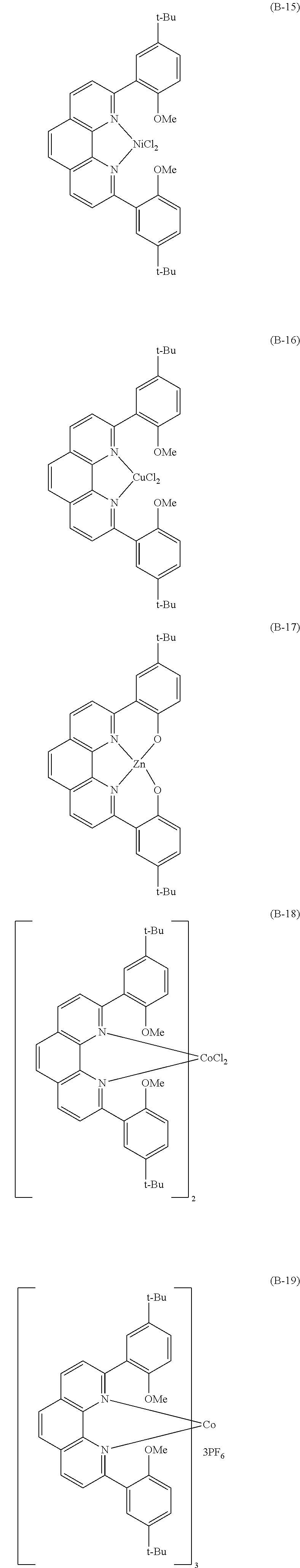

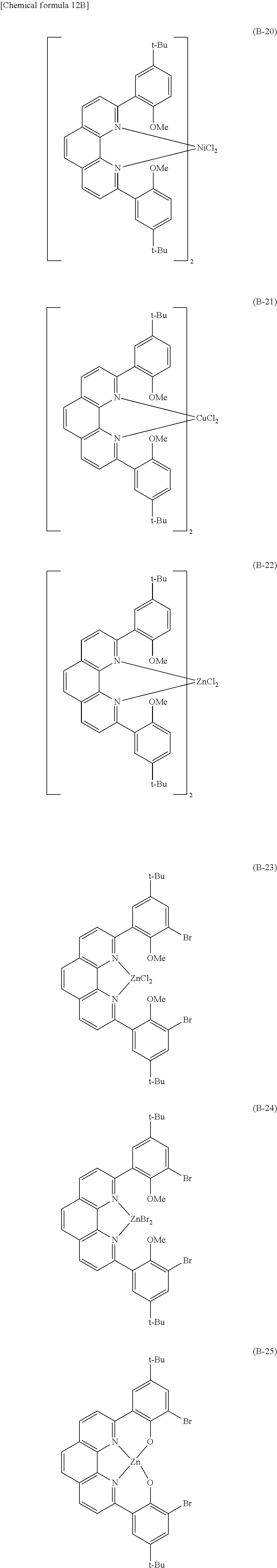

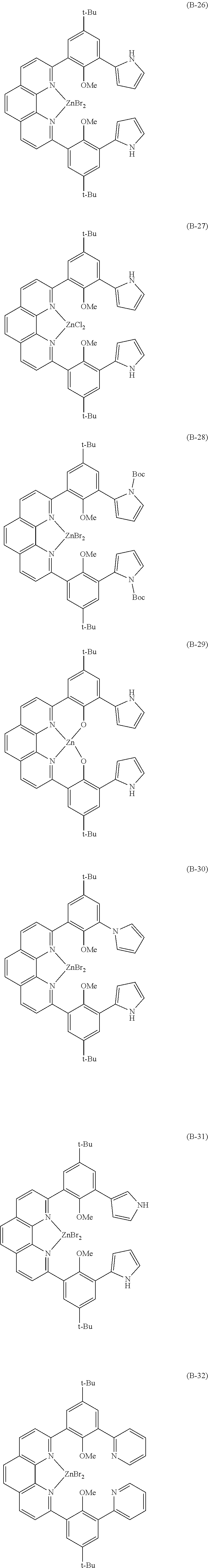

### <Metal Complex 2>

When the metal complex 2 is obtained by performing a halogenation reaction on the metal complex 1, the metal complex 2 is a metal complex in which any one or more of hydrogen atoms of R⁶ to R¹² of the metal complex 1 or any one or more of hydrogen atoms in the substituent (1) when each of R⁶ to R¹² is a substituent are replaced with halogen atoms. When the metal complex 2 is obtained by a pyrrole group introducing reaction on the metal complex 1, the metal complex 2 is a metal complex in which any one or more of halogen atoms of R⁶ to R¹² of the metal complex 1 or any one or more of halogen atoms in the substituent (1) when each of R⁶ to R¹² is a substituent are replaced with pyrrolyl groups optionally having a substituent. When the metal complex 2 is obtained by performing the halogenation reaction and the pyrrole group introducing reaction on the metal complex 1 in this order, the metal complex 2 is a metal complex in which any one or more of hydrogen atoms of R⁶ to R¹² of the metal complex 1 or any one or more of hydrogen atoms in the substituent (1) when each of R⁶ to R¹² is a substituent are replaced with pyrrolyl groups optionally having a substituent.

More specifically, the metal complex 2 is a metal complex generated by adding [M] and [X]_{b} to a bipyridine derivative represented by formula (3) described later.

### <Bipyridine Derivative Represented by Formula (3)>

The bipyridine derivative represented by formula (3) of the present embodiment is a bipyridine derivative (hereinafter, also referred to as a “demetallized product”) obtained by demetallizing a metal from the metal complex 2 in the second step or a bipyridine derivative (hereinafter, also referred to as a “deprotected product”) obtained by deprotecting the demetallized product.

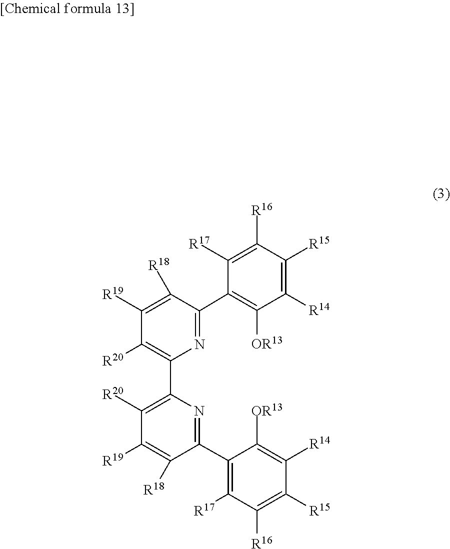

In the formula (3), R¹³ to R²⁰ each independently represent a hydrogen atom or a substituent, R¹³ to R²⁰ may be the same or different, two R¹³s may be the same or different, two R¹⁴s may be the same or different, two R¹⁵s may be the same or different, two R¹⁶s may be the same or different, two R¹⁷s may be the same or different, two R¹⁸s may be the same or different, two R¹⁹s may be the same or different, two R²⁰s may be the same or different, at least one of six Rs consisting of two R¹⁴s, two R¹⁵s, and two R¹⁶s represents a substituent, at least one of two R¹⁷s represents a hydrogen atom, any two substituents of R¹³ to R²⁰ may be bonded to each other to form a ring, each of R¹⁷ to R²⁰ may contain a halogen atom or a pyrrolyl group optionally having a substituent, and at least one of R¹⁴ to R¹⁶ contains a halogen atom or a pyrrolyl group optionally having a substituent.

Specific examples and preferable forms of R¹³ are equivalent to those exemplified for R⁵ in the metal complex 1 represented by the formula (2). Note that when the bipyridine derivative represented by formula (3) of the present embodiment is the deprotected product, two R¹³s are hydrogen atoms.

R¹⁴, R¹⁵, R¹⁶, and R¹⁷ each independently represent a hydrogen atom or a substituent, and specific examples and preferable forms of R¹⁴, R¹⁵, R¹⁶, and R¹⁷ include those exemplified for R⁶, R⁷, R⁸, and R⁹ in the metal complex 1 represented by the formula (2).

At least one of six Rs consisting of two R¹⁴s, two R¹⁵s, and two R¹⁶s is a substituent, and the preferable number of substituents is the form in the R⁶ to R⁸.

At least one of two R¹⁷s is a hydrogen atom. Each of two R¹⁷s is preferably a hydrogen atom.

R¹⁸, R¹⁹, and R²⁰ each independently represent a hydrogen atom or a substituent, and specific examples and preferable forms of R¹⁸, R¹⁹, and R²⁰ include those exemplified for R¹⁰, R¹¹, and R¹² in the metal complex 1 represented by the formula (2).

The plurality of R¹³ to R²⁰ may be the same or different. Any two of R¹³ to R²⁰ may be bonded to each other to form a ring. Preferable examples as to whether the plurality of R¹³, R¹⁴, R¹⁵, R¹⁶, R¹⁷, R¹⁸, R¹⁹, and R²⁰ are the same or different, and preferable examples when any two of R¹³, R¹⁴, R¹⁵, R¹⁶, R¹⁷, R¹⁸, R¹⁹, and R²⁰ are bonded to each other to form a ring are the same as those of the forms in the R⁵, R⁶, R⁷, R⁸, R⁹, R¹⁰, R¹¹, and R¹², respectively.

Each of R¹⁷ to R²⁰ may contain a halogen atom or a pyrrolyl group optionally having a substituent. That is, each of R¹⁷ to R²⁰ may be a halogen atom or a pyrrolyl group optionally having a substituent, and when each of R¹⁷ to R²⁰ is a substituent, the substituent (1) may be a halogen atom or a pyrrolyl group optionally having a substituent.

At least one of R¹⁴ to R¹⁶ contains a halogen atom or a pyrrolyl group optionally having a substituent. In particular, in the formula (3), preferably, any one of R¹⁴ and R¹⁶ is a bipyridine derivative containing a halogen atom or a pyrrolyl group optionally having a substituent, and more preferably, each of two R¹⁴s is a halogen atom or a pyrrolyl group optionally having a substituent.

When R¹⁴ to R²⁰ have a halogen atom, the number of halogen atoms contained in the bipyridine derivative represented by the formula (3) is preferably 1 to 4, and more preferably 1 or 2. When the bipyridine derivative contains a halogen atom, any one or more of R¹⁴ and R¹⁶ are preferably halogen atoms. A halogen atom may be contained in R¹⁴ or R¹⁶ as the substituent (1) when R¹⁴ or R¹⁶ is a substituent.

When R¹⁴ to R²⁰ have a pyrrolyl group optionally having a substituent, the number of pyrrolyl groups optionally having a substituent contained in the bipyridine derivative represented by the formula (3) is preferably 1 to 4, and more preferably 1 or 2. When the bipyridine derivative contains a pyrrolyl group optionally having a substituent, any one or more of R¹⁴ and R¹⁶ are preferably pyrrolyl groups optionally having a substituent. A pyrrolyl group optionally having a substituent may be contained in R¹⁴ or R¹⁶ as the substituent (1) when R¹⁴ or R¹⁶ is a substituent.

The bipyridine derivative represented by the formula (3) may contain a neutral molecule. Examples of the neutral molecule include a molecule that is solvated to form a solvate. Specific examples of the neutral molecule include water, methanol, ethanol, n-propanol, isopropyl alcohol, 2-methoxyethanol, 1,1-dimethylethanol, ethylene glycol, N,N′-dimethylformamide, N,N′-dimethylacetamide, N-methyl-2-pyrrolidone, dimethylsulfoxide, acetone, chloroform, acetonitrile, benzonitrile, triethylamine, pyridine, pyrazine, diazabicyclo [2,2,2] octane, 4,4′-bipyridine, tetrahydrofuran, diethyl ether, dimethoxyethane, methyl ethyl ether, methyl-tert-butyl ether, 1,4-dioxane, acetic acid, propionic acid, and 2-ethylhexanoic acid. Water, methanol, dimethyl sulfoxide, chloroform, tetrahydrofuran, and methyl-tert-butyl ether are preferable.

The bipyridine derivative represented by the formula (3) may form a salt by an acid-base reaction with an acid. Examples of the acid include a molecule that undergoes an acid-base reaction with the bipyridine derivative represented by the formula (3) to form a salt. Specific examples of the acid include hydrochloric acid, bromic acid, iodic acid, phosphoric acid, acetic acid, a sulfate ion, nitric acid, a perchlorate ion, trifluoroacetic acid, a trifluoromethanesulfonate ion, tetrafluoroboric acid, hexafluorophosphoric acid, and tetraphenylboric acid. Among these acids, hydrochloric acid and bromic acid are preferable.

Examples of the bipyridine derivative represented by the formula (3) include bipyridine derivatives represented by the following formulas (C-1) to (C-23) as a structure in which [M] and the [X]_{b} site are eliminated from the metal complexes represented by the formulas (B-2) and (B-23) to (B-25) and the metal complexes represented by the formulas (B-26) to (B-31). In particular, the bipyridine derivatives represented by (C-10) to (C-18) are preferable, and the bipyridine derivatives represented by (C-12) to (C-17) are more preferable.

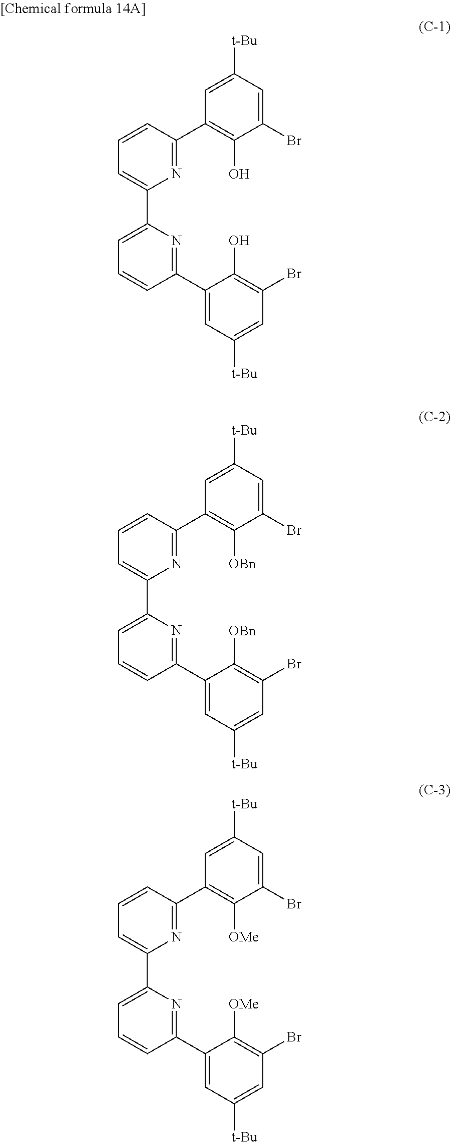

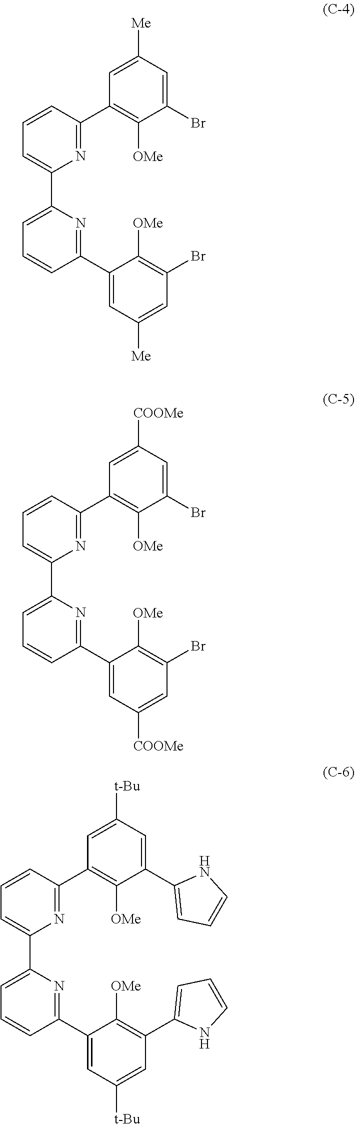

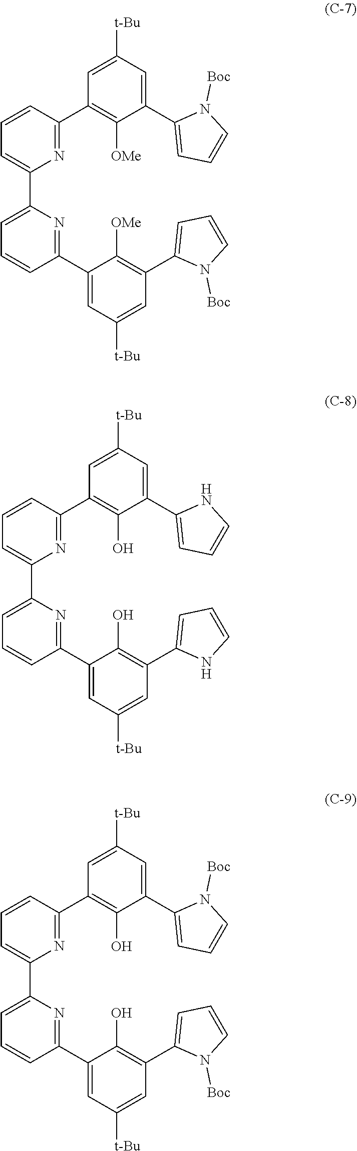 [SMILES: COc1c(-c2cccc(-c3cccc(-c4cc(C(C)(C)C)cc(-c5cccn5C(=O)OC(C)(C)C)c4OC)n3)n2)cc(C(C)(C)C)cc1-c1cccn1C(=O)OC(C)(C)C ; CC(C)(C)c1cc(-c2cccc(-c3cccc(-c4cc(C(C)(C)C)cc(-c5ccc[nH]5)c4O)n3)n2)c(O)c(-c2ccc[nH]2)c1 ; CC(C)(C)OC(=O)n1cccc1-c1cc(C(C)(C)C)cc(-c2cccc(-c3cccc(-c4cc(C(C)(C)C)cc(-c5cccn5C(=O)OC(C)(C)C)c4O)n3)n2)c1O]

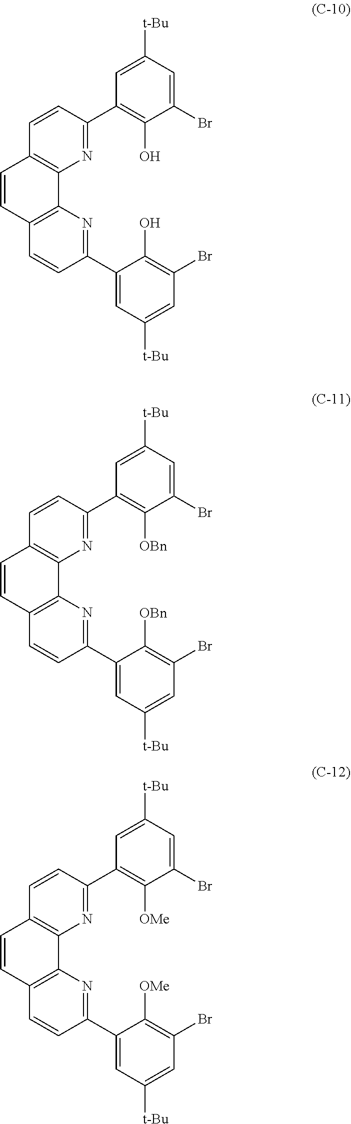

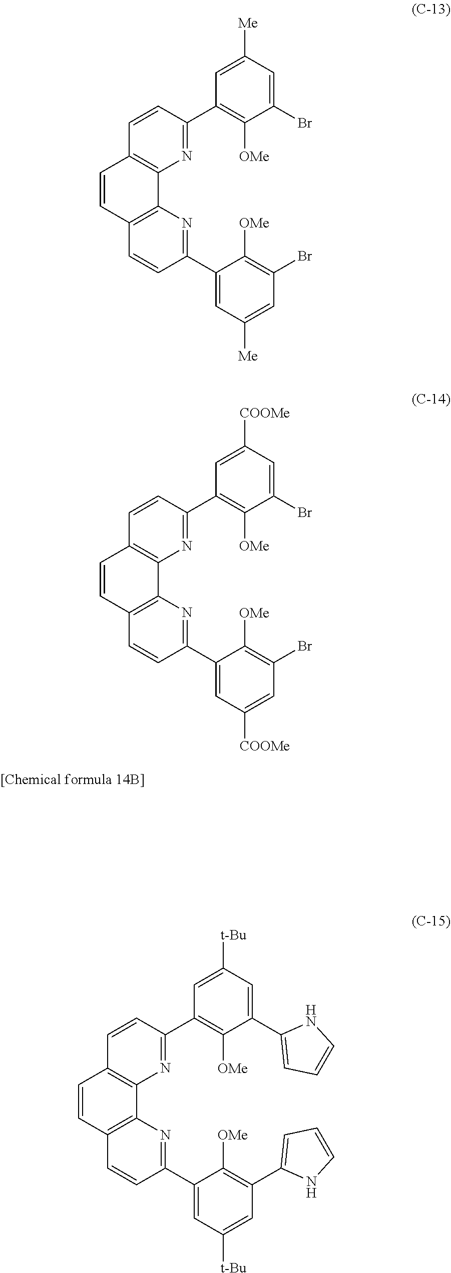 [SMILES: COc1c(Br)cc(C)cc1-c1ccc2ccc3ccc(-c4cc(C)cc(Br)c4OC)nc3c2n1 ; COC(=O)c1cc(Br)c(OC)c(-c2ccc3ccc4ccc(-c5cc(C(=O)OC)cc(Br)c5OC)nc4c3n2)c1 ; COc1c(-c2ccc3ccc4ccc(-c5cc(C(C)(C)C)cc(-c6ccc[nH]6)c5OC)nc4c3n2)cc(C(C)(C)C)cc1-c1ccc[nH]1]

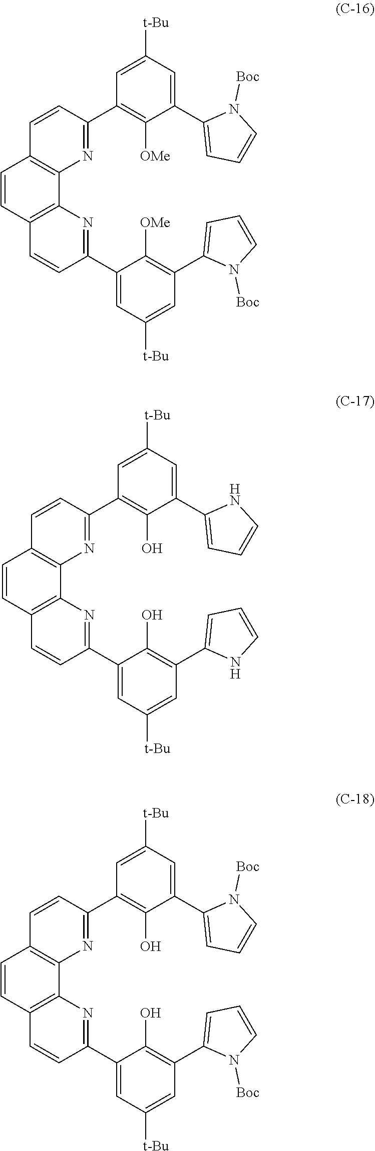 [SMILES: COc1c(-c2ccc3ccc4ccc(-c5cc(C(C)(C)C)cc(-c6cccn6C(=O)OC(C)(C)C)c5OC)nc4c3n2)cc(C(C)(C)C)cc1-c1cccn1C(=O)OC(C)(C)C ; CC(C)(C)c1cc(-c2ccc3ccc4ccc(-c5cc(C(C)(C)C)cc(-c6ccc[nH]6)c5O)nc4c3n2)c(O)c(-c2ccc[nH]2)c1 ; CC(C)(C)OC(=O)n1cccc1-c1cc(C(C)(C)C)cc(-c2ccc3ccc4ccc(-c5cc(C(C)(C)C)cc(-c6cccn6C(=O)OC(C)(C)C)c5O)nc4c3n2)c1O]

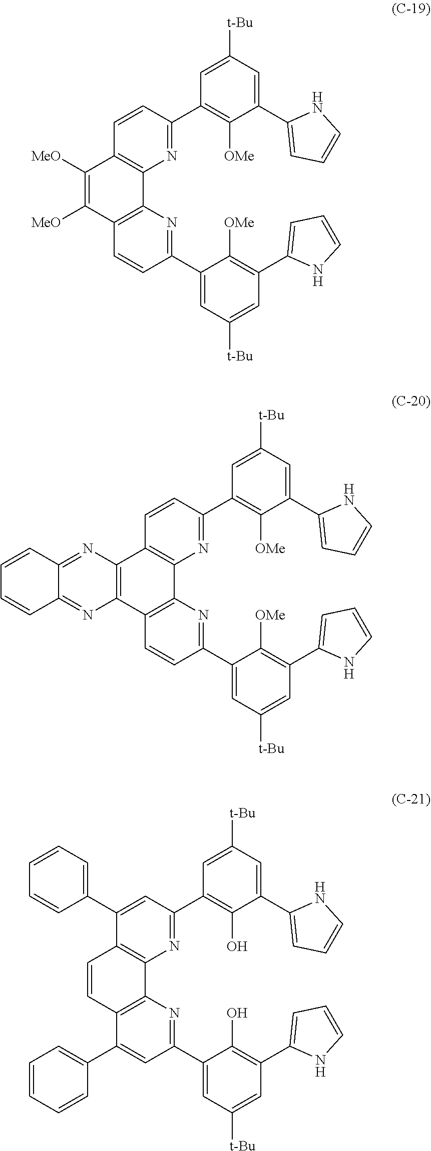 [SMILES: COc1c(-c2ccc3c(OC)c(OC)c4ccc(-c5cc(C(C)(C)C)cc(-c6ccc[nH]6)c5OC)nc4c3n2)cc(C(C)(C)C)cc1-c1ccc[nH]1 ; COc1c(-c2ccc3c(n2)c2nc(-c4cc(C(C)(C)C)cc(-c5ccc[nH]5)c4OC)ccc2c2nc4ccccc4nc32)cc(C(C)(C)C)cc1-c1ccc[nH]1 ; CC(C)(C)c1cc(-c2cc(-c3ccccc3)c3ccc4c(-c5ccccc5)cc(-c5cc(C(C)(C)C)cc(-c6ccc[nH]6)c5O)nc4c3n2)c(O)c(-c2ccc[nH]2)c1]

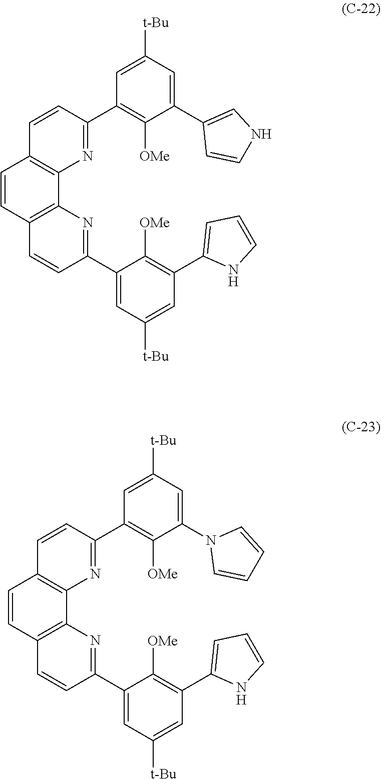 [SMILES: COc1c(-c2cc[nH]c2)cc(C(C)(C)C)cc1-c1ccc2ccc3ccc(-c4cc(C(C)(C)C)cc(-c5ccc[nH]5)c4OC)nc3c2n1 ; COc1c(-c2ccc3ccc4ccc(-c5cc(C(C)(C)C)cc(-n6cccc6)c5OC)nc4c3n2)cc(C(C)(C)C)cc1-c1ccc[nH]1]

In an embodiment of the present invention, the number of halogen atoms contained in the bipyridine derivative represented by the formula (3) is larger than the number of halogen atoms contained in the compound represented by the formula (1). The number of halogen atoms contained in the bipyridine derivative represented by the formula (3) is larger than the number of halogen atoms contained in the compound represented by the formula (1), preferably by 1 to 4, more preferably by 1 or 2. In this case, a halogenation reaction is performed in the second step.

As a preferable embodiment, the compound represented by the formula (1) does not contain a halogen atom, and the bipyridine derivative represented by the formula (3) contains 1 or 2 halogen atoms. In this case, a halogenation reaction is performed in the second step.

In an embodiment of the present invention, the number of pyrrolyl groups optionally having a substituent contained in the bipyridine derivative represented by the formula (3) is larger than the number of pyrrolyl groups optionally having a substituent contained in the compound represented by the formula (1). The number of pyrrolyl groups optionally having a substituent contained in the bipyridine derivative represented by the formula (3) is larger than the number of pyrrolyl groups optionally having a substituent contained in the compound represented by the formula (1), preferably by 1 to 4, more preferably by 1 or 2. In this case, a pyrrole group introducing reaction is performed in the second step, or a halogenation reaction and a pyrrole group introducing reaction are performed in this order.

As a preferable embodiment, the compound represented by the formula (1) does not contain a halogen atom or a pyrrolyl group optionally having a substituent, and the bipyridine derivative represented by the formula (3) contains 1 or 2 pyrrolyl groups optionally having a substituent. In this case, in the second step, the halogenation reaction and the pyrrole group introducing reaction are performed in this order.

As another preferable embodiment, the compound represented by the formula (1) contains 1 or 2 halogen atoms and does not contain a pyrrolyl group optionally having a substituent, and the bipyridine derivative represented by the formula (3) contains 1 or 2 pyrrolyl groups optionally having a substituent. In this case, in the second step, the pyrrole group introducing reaction is performed.

### <First Step>

The first step is a step of obtaining the metal complex 1 represented by the formula (2) from the compound represented by the formula (1).

Specifically, the first step includes a step (hereinafter, also referred to as a “metal complexing step”) of causing the compound represented by the formula (1) to react with a metal salt containing the metal represented by M and the anion species represented by X to obtain the metal complex 1 represented by the formula (2).

Note that the compound represented by the formula (1) can be obtained, for example, by causing a compound represented by the following formula (1′) synthesized by general organic synthesis to react with an oxidizing agent to oxidize an N—H bond in the formula (1′) to generate a bipyridine skeleton. Specifically, the compound represented by the formula (1) can be obtained by mixing the compound represented by the formula (1′) with an oxidizing agent such as manganese dioxide or benzoquinone in a solvent to cause a reaction.

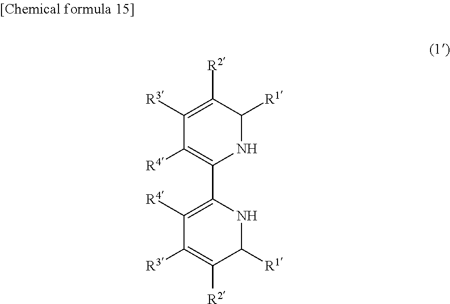

R^{1′} to R^{4′} in the formula (1′) are the same as R¹ to R⁴ in the formula (1), respectively.

### (Metal Complexing Step)

A method used in the metal complexing step is not particularly limited, and a method generally known as a method for converting a bipyridine derivative into a metal complex can be applied. Examples thereof include a method for mixing the compound represented by the formula (1) with a metal salt containing the metal represented by M and the anion species represented by X in a solvent to cause a reaction.

In the present embodiment, in addition to a general-purpose organic solvent, a solvent that is hardly concentrated under reduced pressure is also preferable as the solvent from a viewpoint of crystallinity of a metal complex to be obtained. Specific examples thereof include: an aromatic hydrocarbon-based solvent such as benzene, toluene, xylene, or mesitylene; an ether-based solvent such as diethyl ether, 1,2-dimethoxyethane, methyl ethyl ether, methyl-tert-butyl ether, 1,4-dioxane, tetrahydrofuran, 4-methyltetrahydropyran, or 4-tert-butylanisole; an alcohol-based solvent such as methanol, ethanol, n-propanol, isopropyl alcohol, 2-methoxyethanol, 1-butanol, 1,1-dimethylethanol, or ethylene glycol; a halogen-based solvent such as dichloromethane, chloroform, carbon tetrachloride, chlorobenzene, or 1,2-dichlorobenzene; an amide-based solvent such as N,N′-dimethylformamide, N,N′-dimethylacetamide, or N-methyl-2-pyrrolidone; and a polar solvent such as dimethyl sulfoxide, acetone, or water. A reaction solvent obtained by mixing two or more of these solvents may be used, but a solvent in which the compound represented by the formula (1) and the metal salt can be dissolved is preferable. Among these solvents, an ether-based solvent such as methyl-tert-butyl ether, 4-tert-butyl anisole, 1,4-dioxane, tetrahydrofuran, or 4-methyltetrahydropyran is preferable.

The use amount of the solvent is not particularly limited, and is usually 1 to 200 parts by mass and preferably 3 to 50 parts by mass with respect to 1 part by mass of the compound represented by the formula (1).

The metal salt is not particularly limited as long as it is a compound that can dissociate in a solvent to generate a metal ion.

Examples and preferable forms of the metal ion species capable of forming a metal complex with the compound represented by the formula (1) used in the first step are similar to those described in the M.

Examples of the metal salt that can be dissolved in a solvent to generate a metal ion include a metal salt formed of the metal represented by the M and the anion species represented by the X. Specific examples thereof include organic acid ions such as zinc chloride, zinc bromide, zinc iodide, zinc nitrate, zinc sulfate, zinc perchlorate, zinc acetate, zinc 2-ethylhexanoate, zinc trifluoroacetate, zinc thiocyanate, zinc methanesulfonate, zinc trifluoromethanesulfonate, zinc acetylacetone, zinc tetrafluoroborate, and zinc stearate. Among these salts, zinc chloride, zinc bromide, zinc iodide, and zinc acetate are preferable.

The present reaction may be performed by directly adding the metal salt to a solution in which the compound represented by the formula (1) is dissolved in a solvent, or may be performed by separately preparing a metal salt solution dissolved in a solvent in advance and mixing the metal salt solution with a solution in which the compound represented by the formula (1) is dissolved.

The addition amount of the metal salt is not particularly limited, and it is only required to adjust the amount of the metal salt according to a target metal complex. Usually, the addition amount is 1.0 equivalent or more and 20 equivalents or less, and preferably 1.0 equivalent or more and 5.0 equivalents or less with respect to the compound represented by the formula (1).

Reaction temperature is usually a solidification point of a solvent or higher and a boiling point of the solvent or lower. The reaction temperature is preferably −80 to 100° C., and more preferably −10 to 60° C.

Reaction time is usually one minute to one week, preferably five minutes to 24 hours, and more preferably 30 minutes to 12 hours. Note that the reaction temperature and the reaction time can be appropriately optimized depending on the types of the solvent, the compound represented by the formula (1), and the metal salt.

The metal complex 1 represented by the formula (2), obtained in the metal complexing step can be isolated by crystallization.

The metal complex 1 represented by the formula (2) has higher crystallinity than the compound represented by the formula (1). Therefore, the generated metal complex 1 is precipitated as a solid by appropriately combining operations such as stirring the reaction liquid obtained in the metal complexing step as it is, adding a metal complex as a seed crystal to the reaction liquid obtained in the metal complexing step, partially concentrating the reaction liquid obtained in the metal complexing step, and adding a poor solvent for a metal complex to the reaction liquid obtained in the metal complexing step. At this time, by setting a crystallization temperature to be lower than the reaction temperature in the metal complexing step, crystallization can be efficiently performed, and a target product can be taken out with a high yield.

The crystallization temperature only needs to be a temperature which is lower than the reaction temperature and at which solubility of a metal complex decreases in order to accelerate precipitation of the metal complex as a precipitate, is preferably −80 to 60° C., and more preferably −20 to 40° C.

A method for taking out the metal complex isolated by crystallization is not particularly limited, and examples thereof include solid-liquid separation by filtration or centrifugation. The obtained solid can be isolated and purified by performing a washing operation or a drying operation as necessary.

### <Second Step>

The second step includes: a step of performing one or both of a halogenation reaction (hereinafter, also referred to as a “halogenation step”) and a pyrrole group introducing reaction (hereinafter, also referred to as a “pyrrole group introducing” step) on the metal complex 1 represented by the formula (2) to obtain the metal complex 2; and a demetalation step of demetallizing a metal from the metal complex 2. In addition, a deprotection step may be included after the demetalation step. The case where the second step is a step of performing both the halogenation reaction and the pyrrole group introducing reaction to obtain the metal complex 2 means (i) a step of performing the halogenation reaction on the metal complex 1 to obtain a halogenated product of the metal complex 1, and performing the pyrrole group introducing reaction on the halogenated product of the metal complex 1, or (ii) a step of performing the pyrrole group introducing reaction on the metal complex 1 to obtain a pyrrole group introduced product of the metal complex 1, and halogenating the pyrrole group introduced product of the metal complex 1. Among these steps, (i) a step of performing the halogenation reaction on the metal complex 1 to obtain a halogenated product of the metal complex 1, and performing the pyrrole group introducing reaction on the halogenated product of the metal complex 1 is preferable. Hereinafter, the halogenation step, the pyrrole group introducing step, the demetalation step, and the deprotection step will be described.

### (Halogenation Step)

The halogenation step is a step of causing the metal complex 1 isolated by crystallization after the metal complexing step or the pyrrole group introduced product of the metal complex 1 (hereinafter, the metal complex 1 and the pyrrole group introduced product of the metal complex 1 are also collectively referred to as “metal complex 1-1 or the like”) to react with a halogenating agent to obtain the metal complex 2. When a desired site of the metal complex 1 or the pyrrole group introduced product of the metal complex 1 has already been halogenated, the halogenation step is not necessary.

As a method for causing the metal complex 1-1 or the like to react with a halogenating agent, a method generally known as a method for causing a bipyridine derivative to react with a halogenating agent can be applied. Although not particularly limited, examples thereof include a method for mixing a metal complex with a halogenating agent in a solvent to cause a reaction.

The reaction of the metal complex 1-1 or the like with the halogenating agent can be performed in the presence of an appropriate solvent. Examples of the solvent used in the reaction include: a halogen-based solvent such as dichloromethane, chloroform, or carbon tetrachloride; an ether-based solvent such as tetrahydrofuran or 1,4-dioxane; a nitrile-based solvent such as acetonitrile; an ester-based solvent such as ethyl acetate; an amide-based solvent such as dimethylformamide, and water. A reaction solvent obtained by mixing two or more of these solvents may be used, but a solvent in which a metal complex and a halogenating agent can be dissolved is preferable. Among these solvents, a halogen-based solvent such as dichloromethane, chloroform, or carbon tetrachloride is preferable.

The use amount of the solvent is not particularly limited, and is usually 1 to 200 parts by mass and preferably 3 to 50 parts by mass with respect to 1 part by mass of the metal complex 1-1 or the like.

Examples of the halogenating agent used in the halogenation step include: a halogenating agent that generates free halogen in a reaction system, such as N,N′-bromosuccinimide, N,N′-dibromo-5,5-dimethylhydantoin, or 4-dimethylaminopyridinium bromide perbromide; and bromine (Br₂), and bromine is particularly preferable.

The present reaction can be performed by directly adding a halogenating agent to a solution in which the metal complex 1-1 or the like is dissolved in a solvent. The present reaction can also be performed by separately preparing a halogenating agent solution dissolved in a solvent in advance, and mixing the halogenating agent solution with a solution in which the metal complex 1-1 or the like is dissolved.

The addition amount of the halogenating agent is not particularly limited, and it is only required to adjust the amount of the halogenating agent according to reactivity with the metal complex 1-1 or the like. Usually, the addition amount is 1.0 equivalent or more and 20 equivalents or less, and preferably 1.0 equivalent or more and 10 equivalents or less with respect to the metal complex 1-1 or the like.

Reaction temperature is usually a solidification point of a solvent or higher and a boiling point of the solvent or lower. The reaction temperature is preferably −20 to 100° C., and more preferably 20 to 60° C.

Reaction time is usually one minute to one week, preferably five minutes to 24 hours, and more preferably 30 minutes to 12 hours. Note that the reaction temperature and the reaction time can be appropriately optimized depending on the types of the solvent, the metal complex 1-1 or the like, and the halogenating agent.

It is known that when the halogenation reaction is performed under light irradiation, a bromine radical is generated by photoexcitation of bromine to provide a byproduct, and therefore the halogenation reaction is preferably performed in a dark place.

After completion of the reaction, the excessively added halogenating agent can be quenched by bringing an aqueous solution containing a reducing agent into contact with the solution containing the halogenating agent that has not reacted. Examples of the reducing agent include sodium thiosulfate. The addition amount of the reducing agent is 1.0 equivalent or more and 20 equivalents or less, and preferably 1.0 equivalent or more and 5.0 equivalents or less with respect to the addition amount of the halogenating agent.

Here, the aqueous phase contains hydrogen bromide generated by quenching of the halogenating agent and water-soluble impurities. By removing the aqueous phase by a liquid separation operation and recovering only the organic phase, a halogenated product of the metal complex 1-1 or the like can be taken out from the organic phase.

Usually, hydrogen bromide is produced as a byproduct in a halogenation reaction using a halogenating agent that generates free bromine or bromine. When the present reaction is performed on the bipyridine derivative represented by the formula (3), a nitrogen atom of the bipyridine derivative is protonated by hydrogen bromide produced as a byproduct, and therefore an electron density of the bipyridine derivative decreases. Since the halogenation reaction is an aromatic electrophilic substitution reaction, the bipyridine derivative in which electrons are insufficient due to protonation decreases reactivity and lowers a reaction rate.

Meanwhile, when the present reaction is performed on the metal complex 1-1 or the like, a nitrogen atom in the metal complex 1-1 or the like is coordinated to the [M] component in the formula (2), and an influence of the above-described decrease in the electron density of the bipyridine derivative due to the protonation is small. Therefore, halogenation can be efficiently performed.

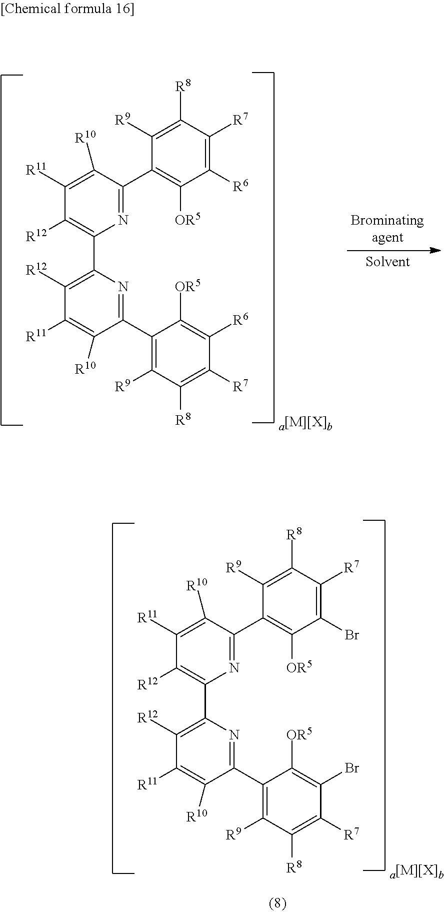

R⁵ to R¹², M, X, a, and b in the formula (8) are the same as those in the formula (2).

The metal complex 2 obtained in the halogenation step can be isolated by crystallization.

The generated metal complex 2 is precipitated as a solid by performing operations such as partially concentrating the organic phase of the reaction liquid obtained in the halogenation step, and adding a poor solvent for the obtained metal complex 2. At this time, crystallization temperature is a temperature which is lower than the reaction temperature and at which solubility of the metal complex 2 decreases in order to accelerate precipitation of the metal complex 2 as a precipitate, is preferably −80 to 60° C., and more preferably −20 to 40° C.

A method for taking out the metal complex 2 is not particularly limited, and examples thereof include solid-liquid separation by filtration or centrifugation. The obtained solid can be isolated and purified by performing a washing operation or a drying operation as necessary.

### (Pyrrole-Group Introducing Step)

The pyrrole group introducing step is a step of performing the pyrrole group introducing reaction on the metal complex 1 isolated by crystallization after the metal complexing step or the halogenated product of the metal complex 1 (hereinafter, the metal complex 1 and the halogenated product of the metal complex 1 are also collectively referred to as “metal complex 1-2 or the like”) to obtain the metal complex 2.

As the pyrrole group introducing reaction, a carbon-carbon and carbon-heteroatom bond formation reaction using a transition metal catalyst, which is known as a method for introducing an olefin such as an aromatic, an alkene, or an alkyne as a substituent into a general organohalogen compound and which is referred to as a cross-coupling reaction, can be applied.

When a halogen atom is present as a substituent in any of R⁶ to R¹² of the metal complex 1 represented by the formula (2), or when the pyrrole group introducing reaction is performed on a halogenated product of the metal complex 1, a pyrrolyl group optionally having a substituent can be introduced specifically by a coupling reaction using palladium as a catalyst typified by a Suzuki-Miyaura coupling reaction, a Mizoroki-Heck reaction, and a Negishi coupling reaction, a coupling reaction using nickel as a catalyst typified by a Yamamoto coupling reaction and a Kumada-Tamao coupling reaction, or a coupling reaction using copper as a catalyst typified by an Ullmann reaction. A coupling reaction using palladium and zinc is preferable.

In particular, as described in a known document (Organic Letters, 2004, 6, 3981), a coupling reaction using palladium and zinc typified by a Negishi coupling reaction is preferable. In the Negishi coupling reaction, by mixing an organohalogen compound and a pyrrole organozinc reagent in a solvent to cause a reaction, a pyrrolyl group optionally having a substituent can be directly introduced without being protected and deprotected.

The method for producing the metal complex 2 by the Negishi coupling reaction generally includes: a step of preparing a pyrrole organozinc reagent (hereinafter, also referred to as a “pyrrole organozinc reagent preparation step”); and a step of mixing the prepared pyrrole organozinc reagent and the metal complex 1-2 or the like in the presence of an appropriate solvent to cause a reaction using a palladium catalyst (hereinafter, also referred to as a “pyrrole reaction step”).

### (Pyrrole Organozinc Reagent Preparation Step)

The pyrrole organozinc reagent preparation step is a step of adding a base and a pyrrole optionally having a substituent to an appropriate solvent to generate a pyrrole anion species, and then adding a zinc salt to prepare a pyrrole organozinc reagent.

The reaction is generally performed under an inert atmosphere such as argon gas under exclusion of oxygen or air in an aprotic solvent until conversion is completed.

Examples of a preferable aprotic solvent include: an ether-based solvent such as diethyl ether, 1,2-dimethoxyethane, methyl ethyl ether, methyl-tert-butyl ether, 1,4-dioxane, tetrahydrofuran, 4-methyltetrahydropyran, or 4-tert-butylanisole; an aromatic hydrocarbon-based solvent such as benzene, toluene, xylene, or mesitylene; a halogen-based solvent such as dichloromethane, carbon tetrachloride, chlorobenzene, or 1,2-dichlorobenzene; an amide-based solvent such as N,N′-dimethylformamide, N,N′-dimethylacetamide, or N-methyl-2-pyrrolidone; and a polar solvent such as dimethyl sulfoxide. A reaction solvent obtained by mixing two or more of these solvents may be used, but a solvent in which the metal complex 1-2 or the like and the pyrrole organozinc reagent can be dissolved is preferable. In particular, tetrahydrofuran is preferable.

The use amount of the solvent is not particularly limited, and is usually 1 to 200 parts by mass and preferably 3 to 50 parts by mass with respect to 1 part by mass of the metal complex 1-2 or the like.

This reaction is preferably performed in the substantial absence of a protic solvent such as water. Unless stated otherwise, the solvent has been dried in order to minimize the presence of a protic solvent such as water. A reaction vessel, a reactant, and a solvent are preferably dried or distilled before use to ensure that no water is present during the reaction.

The base used for generating the pyrrole anion species is not particularly limited, but examples thereof include: a metal hydride such as sodium hydride or potassium hydride; and a metal alkoxide such as sodium methoxide or potassium butoxide, and sodium hydride is preferable.

The addition amount of the base is not particularly limited, and it is only required to adjust the addition amount of the base according to a reaction point of the target metal complex 1-2 or the like, but the addition amount of the base is 1.0 equivalent or more and 10 equivalents or less, and preferably 2.0 equivalents or more and 5.0 equivalents or less with respect to the metal complex 1-2 or the like represented by the formula (2).

The pyrrole optionally having a substituent, used in the pyrrole organozinc reagent preparation step is represented by the following formula (9).

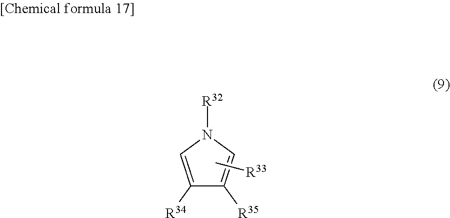

In the formula (9), R³² represents a hydrogen atom or a substituent. When R³² is a substituent, the substituent is preferably a substituent capable of converting an —NR₃₂ site to an —NH structure by converting R³² to hydrogen later, that is, a protecting group. Specific examples of the protecting group in R³² include a tert-butoxycarbonyl group.

R³² is preferably a hydrogen atom.

In the formula, R³³ represents a hydrogen atom or a group represented by “—B (—OY¹)₂”, and is preferably a hydrogen atom.

Y¹ represents a hydrogen atom, an alkyl group having 1 or more and 20 or less carbon atoms, or an aryl group having 6 or more and 20 or less carbon atoms. Two Y¹s may be the same or different, and may be bonded to each other to form a ring.

R³⁴ and R³⁵ each independently represent a hydrogen atom or a substituent. When each of R³⁴ and R³⁵ is a substituent, the substituent is preferably the substituent (1).

Each of R³⁴ and R³⁵ is preferably a hydrogen atom.

Examples of the pyrrole optionally having a substituent, represented by the above formula (9) include pyrroles optionally having a substituent, represented by the following formulas (E1) to (E10).

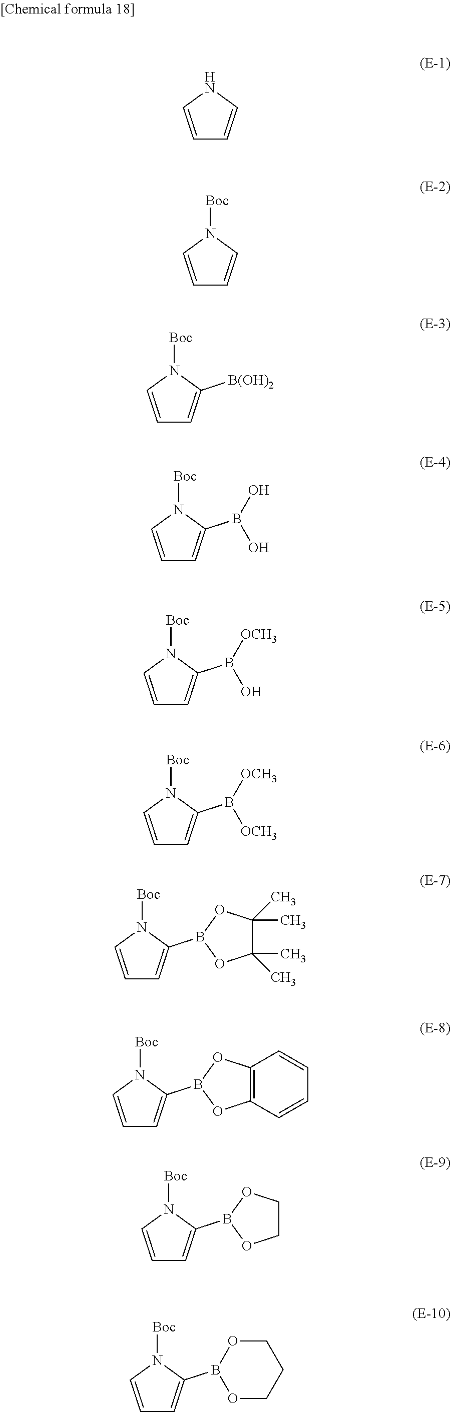

The addition amount of the pyrrole optionally having a substituent is not particularly limited, and it is only required to adjust the addition amount according to a reaction point of the target metal complex 1-2 or the like. The addition amount is 1.0 equivalent or more and 40 equivalents or less, preferably 5.0 equivalents or more and 20 equivalents or less, and more preferably 10 equivalents or more and 20 equivalents or less with respect to the metal complex 1-2 or the like.

Reaction temperature is usually a solidification point of a solvent or higher and a boiling point of the solvent or lower. The reaction temperature is preferably −20 to 100° C., and when a metal hydride is used as the base, the reaction temperature is preferably −20 to 60° C.

The zinc salt is a compound that can dissociate in a solvent to generate a zinc ion. Specifically, the zinc salt is zinc chloride, zinc bromide, or zinc iodide, and may be a hydrate thereof. In particular, zinc chloride or a hydrate thereof is preferable.

The addition amount of the zinc salt is not particularly limited, and it is only required to adjust the amount of the zinc salt according to a target metal complex. The addition amount is 1.0 equivalent or more and 20 equivalents or less, and preferably 2.0 equivalent or more and 8.0 equivalents or less with respect to the metal complex 1-2 or the like.

In the present reaction, the zinc salt may be directly added to a solution in which the pyrrole and the base are dissolved in a solvent to cause a reaction, or the zinc salt dissolved in a solvent prepared separately from the present reaction in advance may be mixed with a solution in which the pyrrole and the base are dissolved.

Reaction time is usually one minute to 24 hours, and preferably five minutes to one hour. Note that the reaction temperature and the reaction time can be appropriately optimized depending on the types of the solvent, the base, and the zinc salt.

### (Pyrrole Reaction Step)

The pyrrole reaction step is a step of mixing a solution containing the pyrrole organozinc reagent prepared in the pyrrole organozinc reagent preparation step with the metal complex 1-2 or the like in the presence of an appropriate solvent to cause a reaction using a palladium catalyst.

The reaction is generally performed under an inert atmosphere such as argon gas under exclusion of oxygen or air in an aprotic solvent until conversion is completed.

Preferable aprotic solvents used in the pyrrole reaction step are similar to those exemplified in the pyrrole organozinc reagent preparation step.

The use amount of the solvent is not particularly limited, and is usually 1 to 200 parts by mass and preferably 3 to 50 parts by mass with respect to 1 part by mass of the metal complex 1-2 or the like.

This reaction is preferably performed in the substantial absence of a protic solvent such as water. Unless stated otherwise, the solvent has been dried in order to minimize the presence of a protic solvent such as water. A reaction vessel, a reactant, and a solvent are preferably dried or distilled before use in order to ensure that no water is present during the reaction.

The palladium catalyst is preferably a complex in which a ligand is coordinated to palladium.

The ligand of palladium is not particularly limited as long as it is a ligand capable of being coordinated to a transition metal, but examples thereof include a phosphorus-based ligand, a nitrogen-based ligand, an oxygen-based ligand, a carbon-based ligand, and an anionic ligand.

The phosphorus-based ligand is not particularly limited as long as it is a ligand having a phosphorus atom capable of being coordinated to a transition metal, but a tertiary phosphine ligand is preferable. Specific examples thereof include triphenylphosphine, tris (2-methylphenyl) phosphine, tris (2-methoxyphenyl) phosphine, di-tert-butylphenylphosphine, tri-tert-butylphosphine, tricyclohexylphosphine, 1,1′-bis (diphenylphosphino) ferrocene (DPPF), 1,3-bis (diphenylphosphino) propane (DPPP), 1,2-bis (diphenylphosphino) ethane (DPPE), 2,2″-bis (diphenylphosphino) −1,1′-binaphthyl (BINAP), 2-dicyclohexylphosphino-2′,6′-dimethoxybiphenyl (SPhos), 2-(dicyclohexylphosphino)-2′,4′,6′-triisopropylbiphenyl (XPhos), 2-(dicyclohexylphosphino)-2′-methylbiphenyl (MePhos), 2-(dicyclohexylphosphino)-2′-(dimethylamino) biphenyl (DavePhos), and 2-(di-tert-butylphosphino) biphenyl (JohnPhos). Note that a quaternary phosphonium salt may be used as the phosphine ligand.

The nitrogen-based ligand is not particularly limited as long as it is a ligand having a nitrogen atom capable of being coordinated to a transition metal, but examples thereof include: a ligand containing a nitrogen-containing aromatic heterocyclic ring such as pyridine, dimethylpyridine, bipyridine, terpyridine, quinoline, isoquinoline, acridine, phenanthroline, N,N-dimethyl-4-aminopyridine (DMAP), or porphyrin, and a salt thereof; an amine-based ligand such as ammonia, aniline, diisopropylamine, 1,1,1,3,3,3-hexamethyldisilazane (HMDS), triethylamine, triphenylamine, 1,8-diazabicyclo [5.4.0] undeca-7-ene (DBU), or N,N,N′,N′-tetramethylethane-1,2-diamine (TMEDA), and a quaternary ammonium salt thereof; and a nitrile-based ligand such as acetonitrile or benzonitrile.

The oxygen-based ligand is not particularly limited as long as it is a ligand having an oxygen atom capable of being coordinated to a transition metal, but examples thereof include: an ether-based ligand such as dimethyl ether, diethyl ether, tetrahydrofuran, 1,4-dioxane, or dimethoxyethane; an alcohol-based ligand such as methanol, ethanol, phenol, or 1,1′-binaphthalene-2,2′-diol; an acyl-based ligand such as acetic acid or acetylacetone; and a phosphine oxide-based ligand such as a phosphate, a phenylphosphonate, a diphenylphosphinate, triphenylphosphine oxide, or trimethylphosphine oxide.

The carbon-based ligand is not particularly limited as long as it is a ligand having a carbon atom capable of being coordinated to a transition metal, but examples thereof include a ligand containing a carbon-carbon multiple bond, such as ethylene, 1-hexene, cyclopentadiene, dibenzylideneacetone (dba), 1,5-cyclooctadiene (COD), or 2-phenylethynylbenzene; an isocyanide-based ligand such as cyanomethyl isocyanide or phenyl isocyanide; a carbene ligand such as N-heterocyclic carbene; and carbon monoxide.

The anion ligand is not particularly limited as long as it is coordinate-bonded to a transition metal with an anionic atomic group. Specific examples of the anionic ligand include: an oxyanionic ligand such as a hydride, a halide ion, a cyanide ion, a methoxy group, a phenoxy group, a phosphate ion, a sulfate ion, a nitrate ion, a trifluoromethanesulfonate, an acetate, or acetylacetonate; and a carbanionic ligand obtained by eliminating a proton from methane, ethane, ethylene, benzene, or the like.

Examples of the palladium catalyst include: a palladium complex such as tetrakis (triphenylphosphine) palladium (0), tris (dibenzylideneacetone) dipalladium (0), palladium (II) acetate, dichlorobistriphenylphosphine palladium (II), or potassium hexachloropalladate (IV); and a complex in which the ligand is coordinated to the palladium complex.

As the palladium catalyst described above, a catalyst synthesized in advance may be used as it is, or a catalyst prepared by adding palladium and a ligand to a solvent prepared separately from the present reaction may be used. Alternatively, palladium and a ligand may be added directly into the reaction system. As the ligand, a phosphorus-based ligand is preferable, and a tertiary phosphine ligand is more preferable. Note that these catalysts may be used singly or in combination of two or more types thereof.

Specific examples of a preferable palladium catalyst used in the pyrrole reaction step include: one prepared by adding a tertiary phosphine ligand selected from the group consisting of 2-dicyclohexylphosphino-2′,6′-dimethoxybiphenyl (SPhos), 2-(dicyclohexylphosphino)-2′,4′,6′-triisopropylbiphenyl (XPhos), and 2-(di-tert-butylphosphino) biphenyl (JohnPhos) to a palladium complex selected from the group consisting of tetrakis (triphenylphosphine) palladium (0) and palladium (II) acetate; and PEPPSI (trademark) -iPr. One prepared by adding 2-(di-tert-butylphosphino) biphenyl (JohnPhos) to palladium (II) acetate is more preferable.

The use amount of the palladium catalyst is not particularly limited, and it is only required to adjust the amount of the palladium catalyst according to a metal complex having a halogen atom as a substituent in any of R⁶ to R¹² of the target metal complex 1-2 or the like, but the use amount of the palladium catalyst is preferably a catalyst amount with respect to the metal complex 1-2 or the like. Specifically, the use amount is 0.001 equivalents or more and 0.5 equivalents or less, and preferably 0.005 equivalents or more and 0.1 equivalents or less with respect to the metal complex 1-2 or the like.

The present reaction can be performed by mixing a solution containing the pyrrole organozinc reagent prepared in the pyrrole organozinc reagent preparation step with a compound having a halogen atom as a substituent in any of R⁶ to R¹² of the metal complex 1 represented by the formula (2) in the presence of an appropriate solvent, and adding the above-described palladium catalyst thereto to cause a reaction.

Reaction temperature is usually a solidification point of a solvent or higher and a boiling point of the solvent or lower. The reaction temperature is preferably −20 to 100° C., and more preferably 0 to 80° C.

Reaction time is usually one minute to 24 hours, and preferably five minutes to 12 hours. Note that the reaction temperature and the reaction time can be appropriately optimized depending on the solvent, the pyrrole organozinc reagent, a case where a halogen atom is present as a substituent in any of R⁶ to R¹² of the metal complex 1 represented by the formula (2) or the halogenated product of the metal complex 1, and the type of the palladium catalyst.

In the formula (2), a case where two R¹s are converted into pyrroles is represented by the following formula (10).

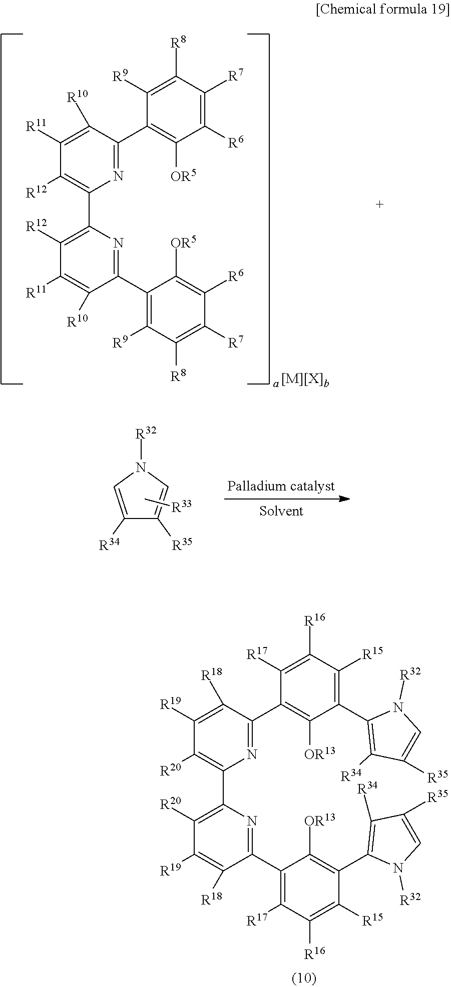

Definitions of R⁵ to R¹², M, X, a, and b in formula (10) are the same as those in the formula (2), definitions of R¹³ to R²⁰ are the same as those in the formula (3), and definitions of R³² to R³⁵ are the same as those in the formula (9). R⁶ is preferably a halogen atom.

The metal complex 2 obtained in the pyrrole group introducing step can be isolated by crystallization.

After completion of the reaction, the generated metal complex 2 is precipitated as a solid by performing operations such as partially concentrating the organic phase containing the metal complex 2, and adding a poor solvent for the obtained metal complex 2. At this time, crystallization temperature is a temperature which is lower than the reaction temperature and at which solubility of the metal complex 2 decreases in order to accelerate precipitation of the metal complex 2 as a precipitate, is preferably −80 to 60° C., and more preferably −20 to 40° C.

A method for taking out the metal complex 2 is not particularly limited, and examples thereof include solid-liquid separation by filtration or centrifugation. The obtained solid can be isolated and purified by performing a washing operation or a drying operation as necessary.

Note that isolation of the metal complex 2 does not have to be performed, and in this case, after completion of the reaction, the organic phase containing the metal complex 2 can be used as the solution in a subsequent step.

### (Demetalation Step)

The demetalation step is a demetalation step in which an acid or a base is added to the metal complex 2 to perform demetalation. By performing demetalation, the bipyridine derivative represented by the formula (3) can be obtained.

In the demetalation step, a method generally known as a method for removing a metal from a metal complex of a bipyridine derivative can be applied, and there is no limitation. However, for example, the demetalation step is a step of dissolving the metal complex 2 in an organic solvent that can be phase-separated from an aqueous phase, bringing the resulting solution into contact with an aqueous solution containing an acid or a base, separating the aqueous phase into which an eliminated metal ion has been extracted and the organic phase from each other by a liquid separation operation, and recovering the organic phase containing the bipyridine derivative represented by the formula (3). The organic phase containing the bipyridine derivative represented by the formula (3) thus obtained may be used as the solution in a subsequent step, or a crystal of the bipyridine derivative represented by the formula (3) may be grown from the organic phase, and a solid may be recovered.

The demetalation step is a step of dissolving the metal complex 2 in an organic solvent that can be phase-separated from an aqueous phase, and bringing the resulting solution into contact with an aqueous solution containing an acid or a base to perform demetalation.

When the solution is brought into contact an aqueous solution containing an acid, an eliminated metal forms a water-soluble metal salt with an anion of the acid, is extracted into the aqueous phase, and can be thereby removed.

When the solution is brought into contact an aqueous solution containing a base, an eliminated metal forms a water-soluble metal salt or a complex ion with an anion of the base, is extracted into the aqueous phase, and can be thereby removed. When an eliminated metal forms a salt that is poorly soluble in water with an anion of the base, the metal can be removed by filtering a precipitate.

Examples of the organic solvent that can be phase-separated from an aqueous phase include: an ether-based solvent such as diethyl ether, 1,2-dimethoxyethane, methyl ethyl ether, methyl-tert-butyl ether, 1,4-dioxane, tetrahydrofuran, or 4-methyltetrahydropyran; an ester-based solvent such as ethyl acetate or butyl acetate; and a halogen-based solvent such as dichloromethane, chloroform, carbon tetrachloride, chlorobenzene, or 1,2-dichlorobenzene, and tetrahydrofuran is preferable. The organic solvent that can be phase-separated from an aqueous phase may be a single solvent or a mixed solvent of a plurality of solvents.

The use amount of the organic solvent that can be phase-separated from an aqueous phase is not particularly limited, and is usually 1 to 200 parts by mass and preferably 3 to 50 parts by mass with respect to 1 part by mass of the metal complex 2.

When phase separation between the organic solvent and the aqueous phase is insufficient and extraction efficiency is poor, it is preferable to appropriately add a salt as a layer separation accelerator to the aqueous phase. Examples of the layer separation accelerator include water-soluble inorganic salts such as sodium chloride, potassium chloride, ammonium chloride, sodium bromide, ammonium bromide, sodium acetate, and ammonium acetate, and sodium chloride and ammonium chloride are preferable from a viewpoint of solubility in the aqueous phase and cost.

Examples of the acid used in the demetalation step include: a hydrogen halide such as hydrogen chloride, hydrogen bromide, or hydrogen iodide; an inorganic acid such as perchloric acid, sulfuric acid, fluorosulfonic acid, nitric acid, phosphoric acid, tetrafluoroboric acid, or hexafluorophosphoric acid; a sulfonic acid such as methanesulfonic acid, ethanesulfonic acid, benzenesulfonic acid, p-toluenesulfonic acid, or trifluoromethanesulfonic acid; and an organic acid such as acetic acid, citric acid, formic acid, gluconic acid, ethylenediaminetetraacetic acid, lactic acid, oxalic acid, tartaric acid, or ascorbic acid.

The amount of the aqueous solution containing an acid is not particularly limited as long as it is an amount by which the metal complex 2 is demetallized to obtain the bipyridine derivative represented by the formula (3), and may be excessive.

Examples of the base used in the demetalation step include: an amine compound such as ammonia, methylamine, N,N,N′,N′-tetramethylethylenediamine, or a water-soluble amine described in the following formula (4); an alkali metal hydroxide such as lithium hydroxide, sodium hydroxide, or potassium hydroxide; an alkaline earth metal hydroxide such as magnesium hydroxide or calcium hydroxide; a quaternary ammonium hydroxide such as tetramethylammonium hydroxide or tetrabutylammonium hydroxide; an alkali metal carbonate such as lithium carbonate, sodium carbonate, or potassium carbonate; an alkali metal bicarbonate such as lithium hydrogen carbonate, sodium hydrogen carbonate, or potassium hydrogen carbonate; and an organic acid alkali metal salt such as sodium citrate, sodium gluconate, sodium ethylenediamine tetraacetate, sodium lactate, sodium oxalate, sodium tartrate, or sodium ascorbate.

As the acid and the base used in the demetalation step, it is desirable to use only the base.

The amount of the aqueous solution containing a base is not particularly limited as long as it is an amount by which the metal complex 2 is demetallized to obtain the bipyridine derivative represented by the formula (3), and may be excessive. When an amine compound, an alkali metal hydroxide, or an alkaline earth metal hydroxide is used, a water-soluble complex ion is formed by using an excessive amount of the amine compound, the alkali metal hydroxide, or the alkaline earth metal hydroxide with respect to the metal complex, and therefore aqueous phase extraction is easy.

Reaction temperature is usually a solidification point of a solvent or higher and a boiling point of the solvent or lower. The reaction temperature is 0 to 100° C., and preferably 10 to 60° C.

Reaction time is usually one minute to 24 hours, and preferably five minutes to one hour. Note that the reaction temperature and the reaction time can be appropriately optimized depending on the types of the solvent, the metal complex 2, the acid, and the base.

Here, the aqueous phase contains a metal ion separated from the metal complex 2 and water-soluble impurities, and the bipyridine derivative represented by the formula (3) can be taken out from the organic phase by removing the aqueous phase by a liquid separation operation and recovering only the organic phase.

### (Water-Soluble Amine)

In the demetalation step, it is preferable to use a water-soluble amine represented by the following formula (4) as a base. By using the water-soluble amine, an eliminated metal ion forms a metal ammine complex having high water solubility, and therefore aqueous phase extraction of the metal ion is easy.

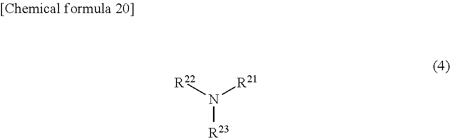

In the formula (4), R²¹ to R²³ each independently represent a hydrogen atom or a substituent.

The substituents are preferably one or more substituents selected from the group consisting of a methyl group, an ethyl group, a hydroxymethyl group, and a hydroxyethyl group. Each of R²¹, R²², and R²³ is preferably a hydrogen atom. R²¹, R²², and R²³ may be the same or different, but are preferably the same.

The amount of the aqueous solution containing the water-soluble amine is not particularly limited as long as it is an amount by which the metal complex 2 is demetallized to obtain the bipyridine derivative represented by the formula (3), and may be excessive. When an amine compound, an alkali metal hydroxide, or an alkaline earth metal hydroxide is used as the base, a water-soluble complex ion is formed by using an excessive amount of the amine compound, the alkali metal hydroxide, or the alkaline earth metal hydroxide with respect to the metal complex, and therefore aqueous phase extraction is easy.

Examples of the water-soluble amine represented by the formula (4) include water-soluble amines represented by following formulas (D-1) to (D-19). Among these amines, water-soluble amines represented by (D-1), (D-2), and (D-5) are preferable, and water-soluble amines represented by (D-1) and (D-2) are more preferable.

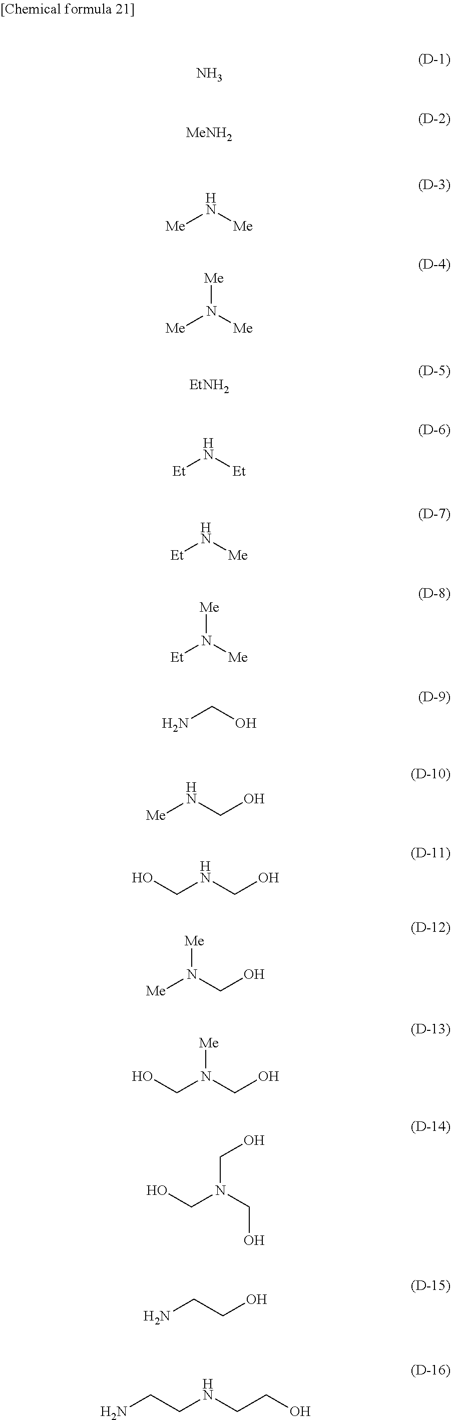

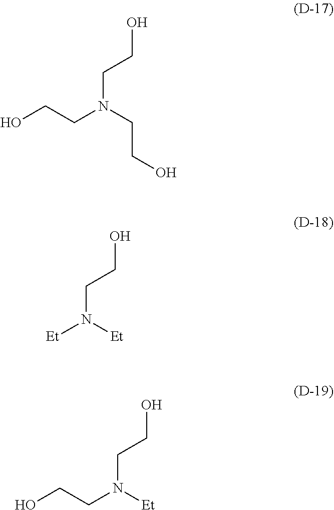 [SMILES: OCCN(CCO)CCO ; CCN(CC)CCO ; CCN(CCO)CCO]

The demetallized product obtained in the demetalation step can be isolated by crystallization.

The organic phase recovered in the demetalation step is concentrated under reduced pressure, a poor solvent is added thereto as necessary, crystallization is performed, and the demetallized product can be thereby recovered as a solid.

Crystallization temperature is a temperature at which solubility of the target product decreases in order to accelerate precipitation of the target product as a precipitate, and is preferably −80 to 60° C. and more preferably −20 to 40° C.

A method for taking out the target product is not particularly limited, and examples thereof include solid-liquid separation by filtration or centrifugation. The obtained solid can be isolated and purified by performing a washing operation or a drying operation as necessary.

Note that the isolation step of the demetallized product does not have to be performed, and in this case, after completion of the reaction, the organic phase containing the demetallized product can be used as the solution in a subsequent step.

### (Deprotection Step)

The deprotection step is a step of deprotecting a protecting group when the demetallized product has the protecting group. Specifically, the deprotection step is a step of causing the bipyridine derivative represented by the formula (3), obtained by the demetalation step to react with a deprotecting agent to convert an —OR¹³ site of the bipyridine derivative represented by the formula (3) into an —OH structure to obtain a deprotected product.

As described in JP-B2-5422159 and a known document (Arch. Pharm. Res. 2008, 31, 305), a method known as a method for deprotecting a general protecting group of an aryl hydroxy group can be applied to a method for producing the deprotected product.

In the formula (10), an N—R³² site may be deprotected to be converted into an N—H structure.

In the formula (10), when the protecting group in R³² is a tert-butoxycarbonyl group, boron tribromide or the like which is generally known as a method for deprotecting the tert-butoxycarbonyl group can be applied.

In the formula (3), either of R¹³s is preferably deprotected, and two R¹³s are more preferably deprotected. A case where two R¹³s are deprotected is represented by the following formula (11).

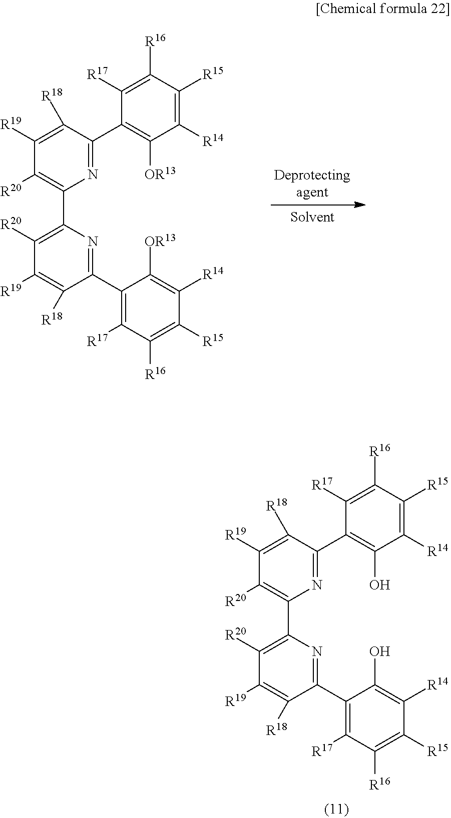

Definitions of R¹³ to R²⁰ in the formula (11) are the same as those in the formula (3). Note that, in this case, R¹³ is the protecting group described above.

The deprotected product obtained in the deprotection step can be isolated by crystallization.

The organic phase recovered in the deprotection step is concentrated under reduced pressure, a poor solvent is added thereto as necessary, crystallization is performed, and the deprotected product can be thereby recovered as a solid.

Crystallization temperature is a temperature at which solubility of the target product decreases in order to accelerate precipitation of the target product as a precipitate, and is preferably −80 to 60° C. and more preferably −20 to 40° C.

A method for taking out the target product is not particularly limited, and examples thereof include solid-liquid separation by filtration or centrifugation. The obtained solid can be isolated and purified by performing a washing operation or a drying operation as necessary.

### Action and Mechanism

According to the method for producing a bipyridine derivative of the present embodiment, an intermediate taken out in an intermediate step for producing the bipyridine derivative represented by the formula (3) using the compound represented by the formula (1) as a starting raw material can be isolated by crystallization and filtration purification without requiring purification by column chromatography. Therefore, a bipyridine derivative can be produced at a high yield. Furthermore, a bipyridine derivative can be produced with high purity by crystallization purification.

Meanwhile, as described above, in the method described in Patent Document 1, it is difficult to isolate an intermediate by crystallization and filtration purification. A reason for this is considered as follows. In the method described in Patent Document 1, when the bipyridine derivative represented by the formula (3) is synthesized, since an intermediate to be taken out in an intermediate step has low crystallinity, the intermediate is likely to be oily without being solidified even when concentrated in the presence of a reactant introduced in an excessive amount with respect to the compound represented by the formula (1) or solvent-soluble impurities produced as a byproduct by the reaction. In addition, even when a poor solvent is added after concentration and recrystallization is attempted, impurities are likely to be precipitated.

Meanwhile, when the amount of a good solvent to be introduced is increased in order to avoid precipitation of impurities, the target product having low crystallinity and high solubility is dissolved, and a yield decreases. In particular, this tendency is significant when a reactant itself introduced in an excess amount with respect to the compound represented by the formula (1) is a good solvent for the bipyridine derivative represented by the formula (3). Therefore, it is difficult to recover the target product with high purity at a high yield by crystallization and filtration purification, and purification by column chromatography is required.

On the other hand, according to the method for producing a bipyridine derivative of the present embodiment, crystallinity can be improved by adding a metal salt and using the metal complex 1 as an intermediate in the first step. Meanwhile, since the reactant introduced in an excessive amount and impurities hardly form a metal complex, the target product can be selectively metal-complexed. As a result, it is not necessary to add a poor solvent or a good solvent in the crystallization step, and it is possible to avoid a decrease in purity due to impurity precipitation and a decrease in yield due to dissolution of the target product, and to recover the target product with high purity at a high yield.

Next, the metal complex 1 generated in the first step is isolated, and isolation of the metal complex 1 can be usually performed by crystallization and solid-liquid separation by filtration. By separating the liquid by this isolation, it is possible to remove, into the liquid, a reactant that does not form a complex and introduced in an excessive amount, and solvent-soluble impurities produced as a byproduct by the reaction. In addition, isolation of the metal complex 2 similarly produced in the second step can be usually performed by crystallization and solid-liquid separation by filtration.

Next, in the demetalation step, an acid or a base is added to the metal complex 2 to demetallize the metal complex 2, and the bipyridine derivative represented by the formula (3) can be thereby obtained. Since the metal complex 2 is used as a starting raw material in the demetalation step, a high purity bipyridine derivative can be obtained with high purity by performing the demetalation step.

In the method for producing a bipyridine derivative of the present embodiment, selection of the metal M is important. In the present embodiment, the metal M is any metal belonging to Groups 4 to 12 in the fourth period of the periodic table. Such a metal M has characteristics that crystallinity of a metal complex is improved and the metal M is easily eliminated in the demetalation step because the valence of the metal M is hardly changed in the method for producing a bipyridine derivative of the present embodiment. Therefore, it is considered that a high-purity bipyridine derivative can be obtained with high purity only by selecting any metal belonging to Groups 4 to 12 in the fourth period of the periodic table as the metal M.

### <<Metal Complex>>

The metal complex of the present embodiment is a metal complex represented by the following formula (6).

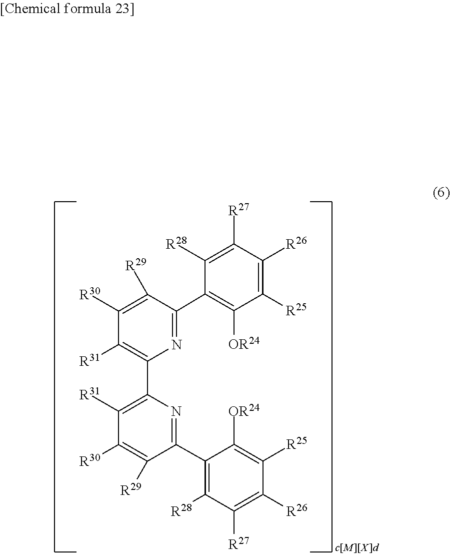

In the formula (6), R²⁴ represents a substituent, R²⁵ to R³¹ each independently represent a hydrogen atom or a substituent, R²⁴ to R³¹ may be the same or different, two R²⁴s may be the same or different, two R²⁵s may be the same or different, two R²⁶s may be the same or different, two R²⁷s may be the same or different, two R²⁸s may be the same or different, two R³⁰s may be the same or different, two R³¹s may be the same or different, at least one of six Rs consisting of two R²⁵s, two R²⁶s, and two R²⁷s represents a substituent, at least one of two R²⁸s represents a hydrogen atom, any two organic groups of R²⁴ to R³¹ may be bonded to each other to form a ring, M represents any metal belonging to Groups 4 to 12 in the fourth period of the periodic table, X represents an anion species, c represents an integer of 1 to 3, and d represents 0 or more.

A difference between the metal complex 1 represented by the formula (2) and the metal complex represented by the formula (6) is that R⁵ is a hydrogen atom or a substituent in the metal complex 1 represented by the formula (2), whereas R²⁴ is a substituent in the metal complex represented by the formula (6).

In the metal complex represented by the formula (6), the substituent is preferably a substituent capable of converting an —OR²⁴ site to an —OH structure by converting the substituent of R²⁴ to hydrogen, that is, a protecting group. Specific examples of the protecting group in R²⁴ are the same as those in R⁵ in the formula (2).

R²⁵ to R²⁷ each independently represent a hydrogen atom or a substituent. Specific examples and preferable forms of R²⁵, R²⁶, and R²⁷ are equivalent to those of R⁶, R⁷, and R⁸ in the metal complex 1 represented by the formula (2), respectively. At least one of six Rs consisting of two R²⁵s, two R²⁶s, and two R²⁷s is a substituent, and preferable forms of the number of substituents in R²⁵, R²⁶, and R²⁷ are also equivalent to those in R⁶, R⁷, and R⁸, respectively.

R²⁸ represents a hydrogen atom or a substituent, and specific examples and preferable forms of R²⁸ are equivalent to those of R⁹ in the metal complex 1 represented by the formula (2). At least one of two R²⁸s is a hydrogen atom, and preferable forms of the number of substituents in R²⁸ are also equivalent to those in the R⁹.

R²⁹ to R³¹ each independently represent a hydrogen atom or a substituent. Specific examples and preferable forms of R²⁹, R³⁰, and R³¹ are equivalent to those of R¹⁰, R¹¹, and R¹² in the metal complex 1 represented by the formula (2), respectively.

The plurality of R²⁹ to R³¹ may be the same or different. Any two of R²⁹ to R³¹ may be bonded to each other to form a ring. Preferable forms as to whether R²⁹, R³⁰, and R³¹ are the same and as to whether R²⁹, R³⁰, and R³¹ are bonded to each other to form a ring are also equivalent to those of R¹⁰, R¹¹, and R¹², respectively.

c represents an integer of 1 to 3. A preferable form of c is the same as that of a in the metal complex 1 represented by the formula (2).

M represents a metal. Specific examples and preferable forms of M are the same as those of M in the metal complex 1 represented by the formula (2).

d represents the number of Xs in the metal complex, and represents 0 or more. A preferable form of d is the same as that of b in the metal complex 1 represented by the formula (2).

The metal complex represented by the formula (6) can be produced by performing the operation of the first step using the compound represented by the formula (1) as a raw material. When the metal complex represented by the formula (6) contains a halogen atom and the number of halogen atoms in the metal complex represented by the formula (6) is larger than the number of halogen atoms contained in the compound represented by the formula (1), the metal complex represented by the formula (6) can be produced by performing an operation of a halogenation step in addition to the first step. When the metal complex represented by the formula (6) contains a pyrrolyl group optionally having a substituent and the number of pyrrolyl groups optionally having a substituent in the metal complex represented by the formula (6) is larger than the number of pyrrolyl groups optionally having a substituent contained in the compound represented by the formula (1), the metal complex represented by the formula (6) can be produced by performing an operation of a pyrrole group introducing step or performing a halogenation step and the pyrrole group introducing step in this order in addition to the first step.

Examples of the metal complex represented by the formula (6) include metal complexes represented by following formulas (F-1) to (F-32). Among these metal complexes, examples of the halogenated product of the metal complex represented by the formula (6) are metal complexes represented by formulas (F-2) and formulas (F-23) to (F-25), and examples of the pyrrole group introduced product of the metal complex represented by the formula (6) are metal complexes represented by formula (F-26) to (F-31). Among these metal complexes, formulas (F-1) to (F-13), (F-17), and (F-22) to (F-32) in which M is zinc are preferable.

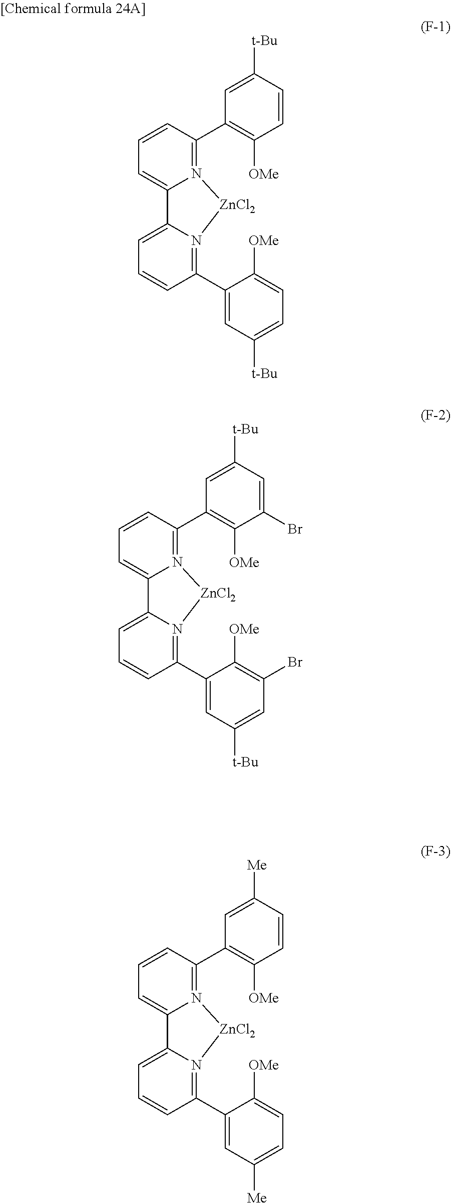

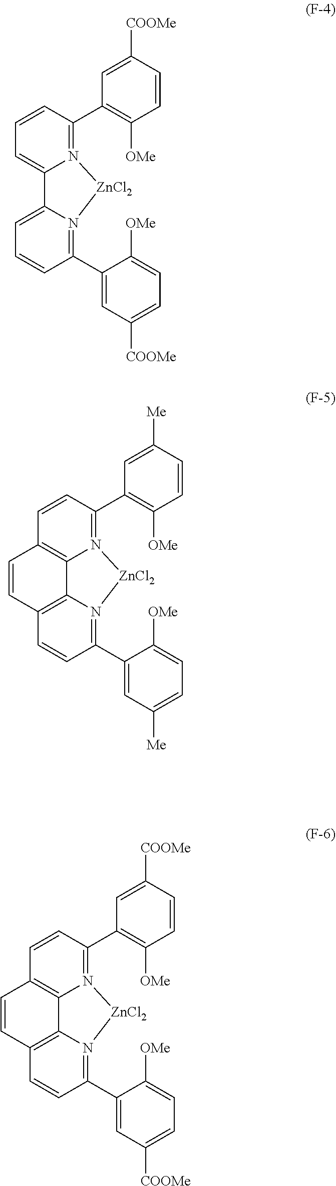

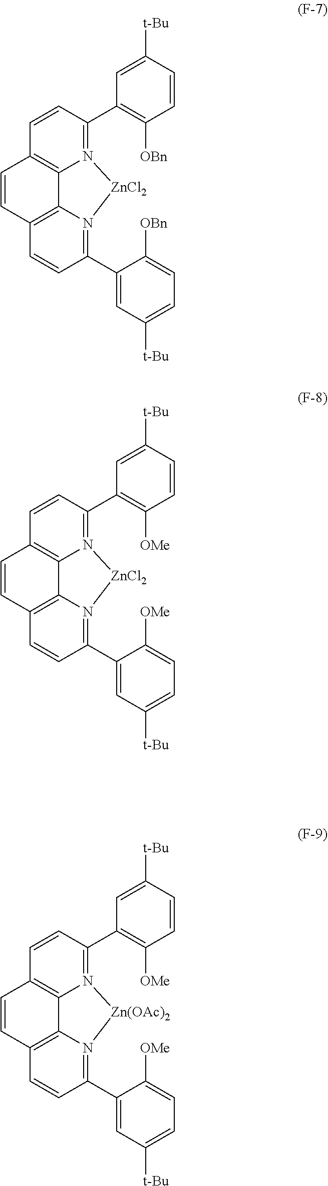

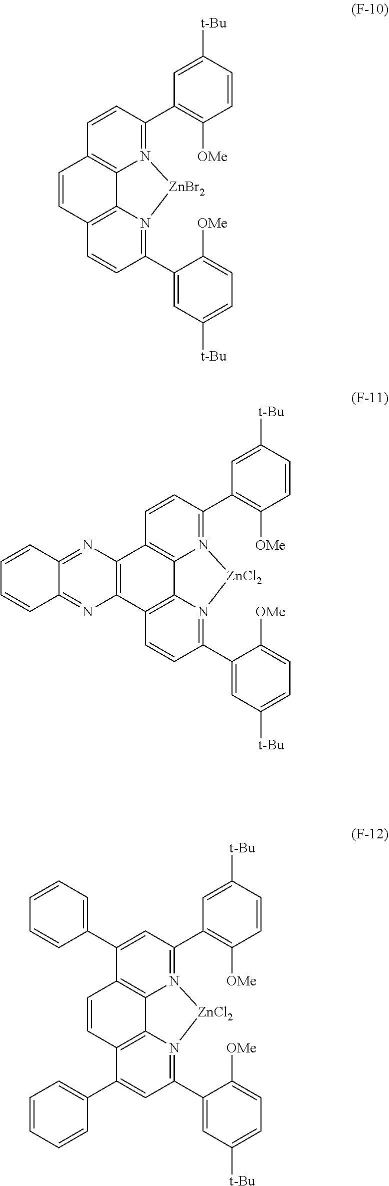

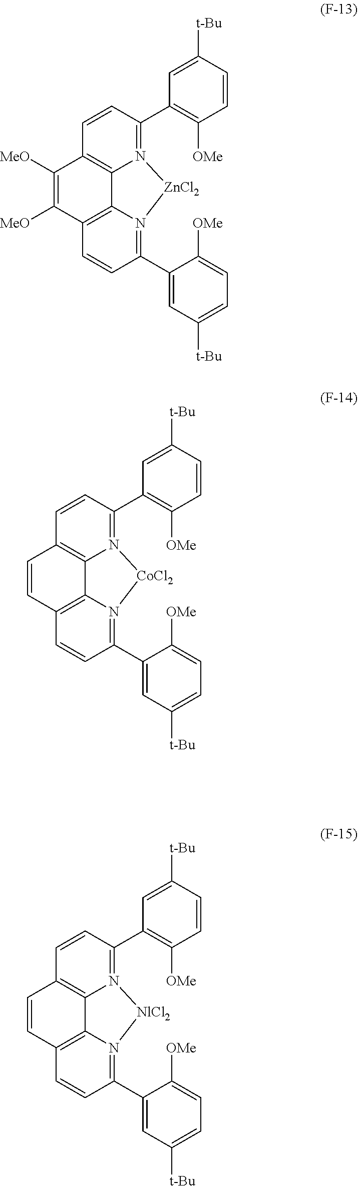

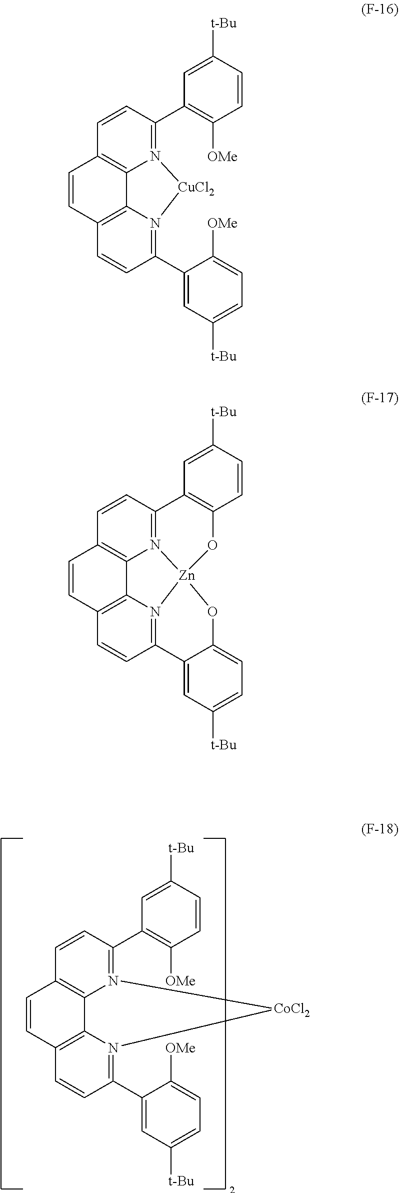

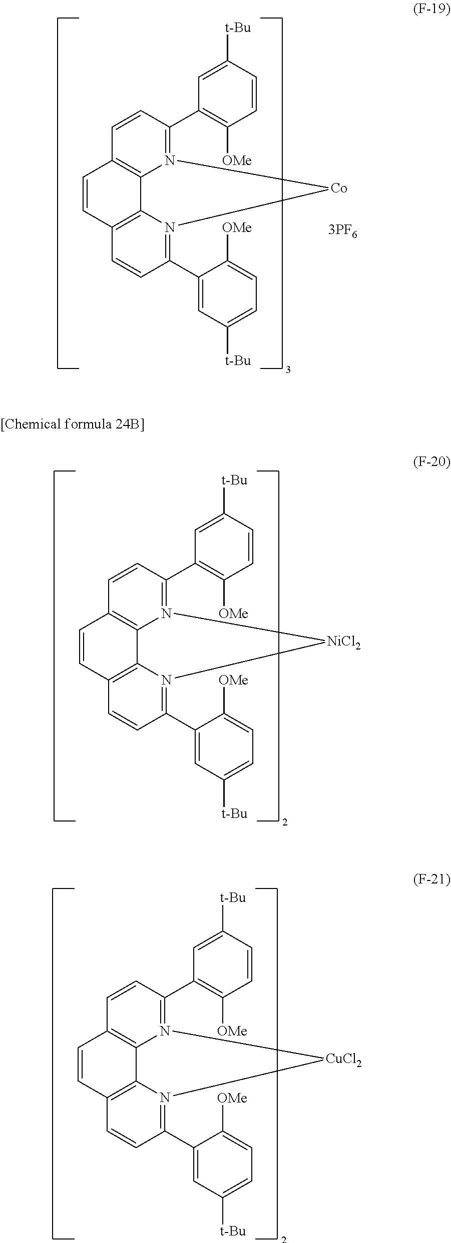

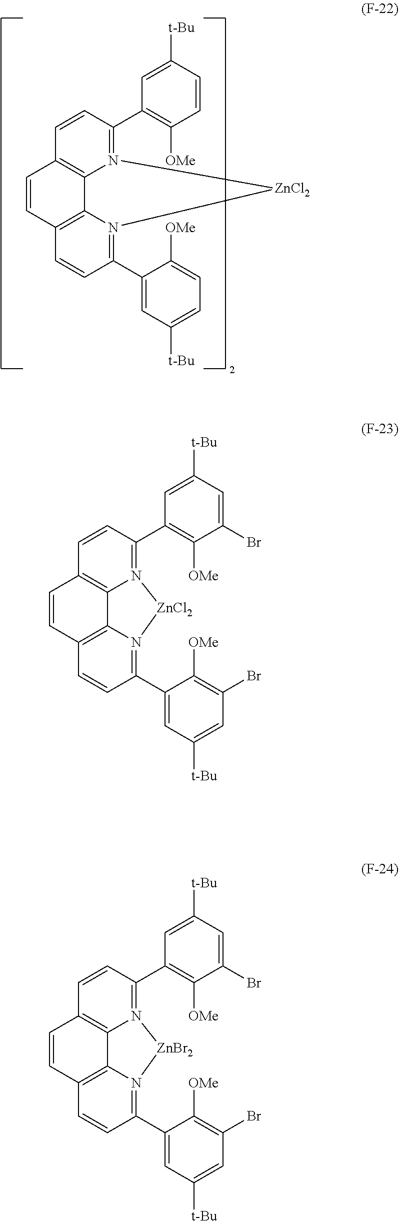

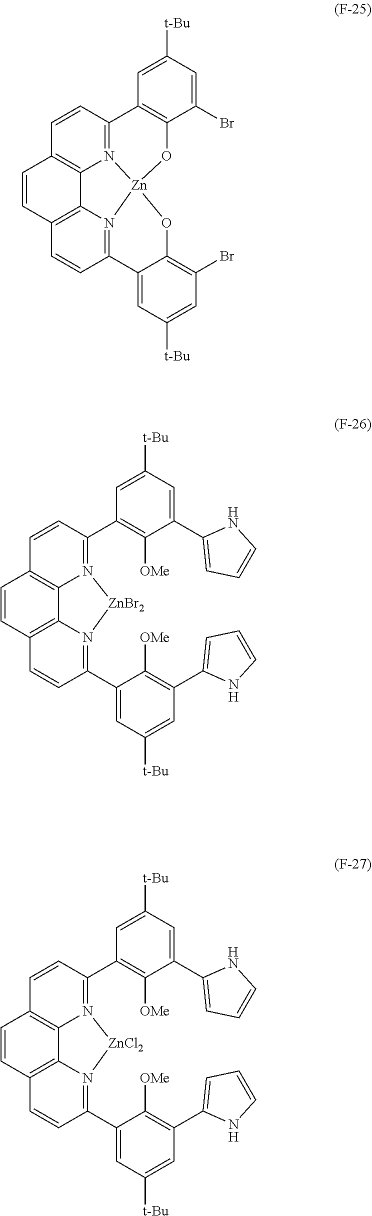

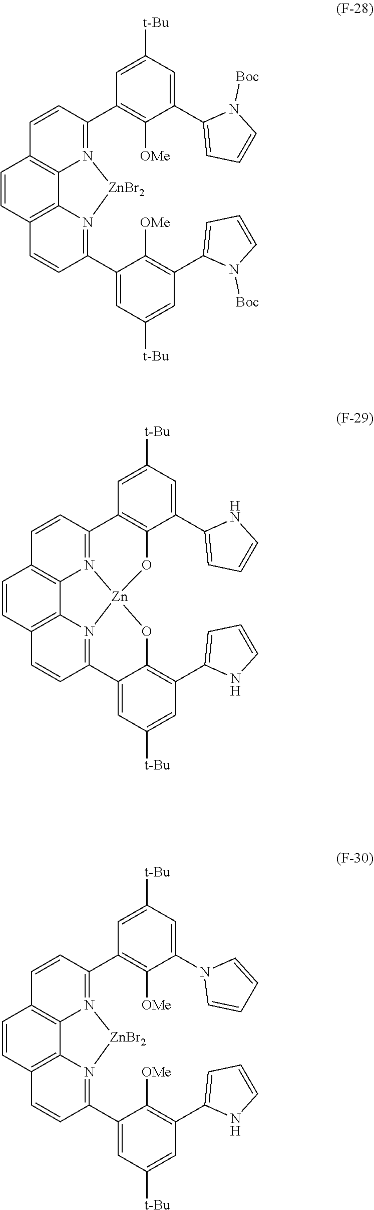

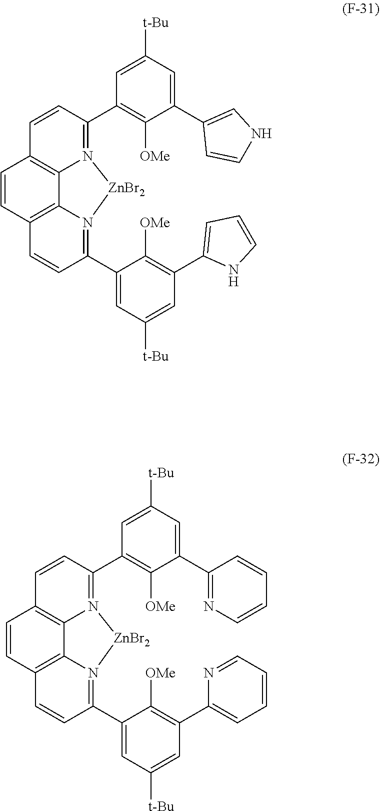

### <<Method for Producing Macrocyclic Compound>>

A method for producing a macrocyclic compound of the present embodiment is a method for producing a macrocyclic compound, the method including ring-closing the bipyridine derivative having two or more pyrrolyl groups optionally having a substituent, which has been produced by the above production method and is represented by the formula (3), to obtain a macrocyclic compound represented by the following formula (5).

In the present specification, the “macrocyclic compound” means a compound which has five or more aromatic rings and in which atoms constituting ring skeletons of these five or more aromatic rings further form a macrocyclic skeleton having a larger number of ring members (number of atoms constituting the ring skeleton) than the number of ring members of each of these aromatic rings. Here, the “atoms constituting the ring skeleton” are, for example, four carbon atoms and one nitrogen atom in a case of a pyrrole ring, and a total of five hydrogen atoms bonded to these carbon atoms and nitrogen atom are not atoms constituting the ring skeleton.

In the present specification, the “aromatic ring” includes a heteroaromatic ring in which at least one of atoms constituting a ring skeleton is a heteroatom (for example, a nitrogen atom).

In addition, in the present specification, as described above, the “macrocyclic skeleton” means not an aromatic ring having a smaller number of ring members than the aromatic ring but a ring skeleton constituted by the aromatic ring and having a larger number of ring members than the aromatic ring.

Note that, in the present specification, for example, a ring structure in which two or more aromatic rings are condensed, such as a benzotriazole ring, a naphthalene ring, or a phenanthroline ring, is treated as one aromatic ring. In a case of a phenanthroline ring, 12 carbon atoms and two nitrogen atoms are atoms forming a ring skeleton.

The method for producing a macrocyclic compound of the present embodiment includes a step (hereinafter, also referred to as “step 3-1”) of performing an intramolecular cyclization reaction by, for example, causing the bipyridine derivative having two or more pyrrolyl groups optionally having a substituent, represented by the formula (3) to react with a compound having an aldehyde group to obtain a precursor of a macrocyclic compound.

The method for producing a macrocyclic compound of the present embodiment further includes a step (hereinafter, also referred to as “step 3-2”) of performing an oxidation reaction by causing the precursor of the macrocyclic compound obtained in step 3-1 to react with an oxidizing agent or the like to obtain the macrocyclic compound.

Hereinafter, the macrocyclic compound represented by the following formula (5) in the present embodiment will be described. In addition, manufacturing conditions will be described.

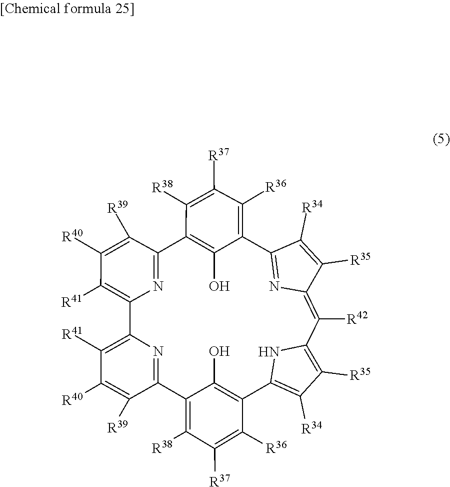

In the formula (5), R³⁴ to R⁴² each independently represent a hydrogen atom or a substituent, the plurality of R³⁴ to R⁴² may be the same or different, and any two substituents of R³⁴ to R⁴² may be bonded to each other to form a ring.

Specific examples and preferable forms of R³⁶, R³⁷, and R³⁸ are the same as those of the substituents described for R⁷, R⁸, and R⁹ in the metal complex 1 represented by the formula (2), respectively.

In the four Rs consisting of two R³⁶s and two R³⁷s, the total number of substituents is 0 to 4, preferably 0 to 2, and more preferably 2.

R³⁸ represents a hydrogen atom or a substituent, and specific examples and preferable forms of R³⁸ are equivalent to those exemplified for R⁹ in the metal complex 1 represented by the formula (2). In two R³⁸s, the possible number of substituents is 0 or 1, and two R³⁸s are preferably hydrogen atoms.

R³⁹, R⁴⁰, and R⁴¹ each independently represent a hydrogen atom or a substituent, and specific examples and preferable forms of R³⁹, R⁴⁰, and R⁴¹ are equivalent to those exemplified for R¹⁰, R¹¹, and R¹² in the metal complex 1 represented by the formula (2), respectively.

The plurality of R³⁶ to R⁴¹ may be the same or different. Any two of R³⁶ to R⁴¹ may be bonded to each other to form a ring. Preferable examples as to whether the plurality of R³⁶, R³⁷, R³⁸, R³⁹, R⁴⁰, and R⁴¹ are the same or different, and preferable examples when any two of R³⁶, R³⁷, R³⁸, R³⁹, R⁴⁰, and R⁴¹ are bonded to each other to form a ring are the same as those of the forms in the R⁷, R⁸, R⁹, R¹⁰, R¹¹, and R¹², respectively.

R⁴² represents a hydrogen atom or a hydrocarbyl group having 1 to 30 carbon atoms and optionally having a substituent. Examples of the hydrocarbyl group represented by the substituent R⁴² include an alkyl group, an aryl group, and an aralkyl group, and an alkyl group and an aryl group are preferable.

Exemplification of the alkyl group, the aryl group, and the aralkyl group are the same as that for R¹ to R⁴ in the formula (1).

R⁴² is preferably a phenyl group optionally having a substituent, more preferably a phenyl group optionally having a substituent of a hydrocarbyl group having 1 to 30 carbon atoms, and still more preferably a phenyl group optionally having a substituent of an alkyl group having 1 to 8 carbon atoms.

The macrocyclic compound represented by the formula (5) is preferably a compound in which the macrocyclic skeleton is constituted by 5 or more and 12 or less aromatic rings, and more preferably a compound in which the macrocyclic skeleton is constituted by five aromatic rings including a phenanthroline ring.

The macrocyclic compound represented by the formula (5) preferably has four or more nitrogen atoms as atoms capable of being coordinated, preferably has four or more and six or less nitrogen atoms as atoms capable of being coordinated, and more preferably has four nitrogen atoms and two oxygen atoms as atoms capable of being coordinated.

In the macrocyclic compound represented by the formula (5), the minimum number of atoms constituting the maximum ring skeleton (the number of atoms constituting an inner periphery of the macrocyclic skeleton) is preferably 9 to 50, more preferably 16 to 33, still more preferably 17 to 32, and particularly preferably 19 or 20.

Examples of the macrocyclic compound represented by the formula (5) include macrocyclic compounds represented by following formulas (G-1) to (G-16). Among these compounds, the macrocyclic compounds represented by (G-1) to (G-8) are preferable, and the macrocyclic compounds represented by (G-5) and (G-6) are more preferable.

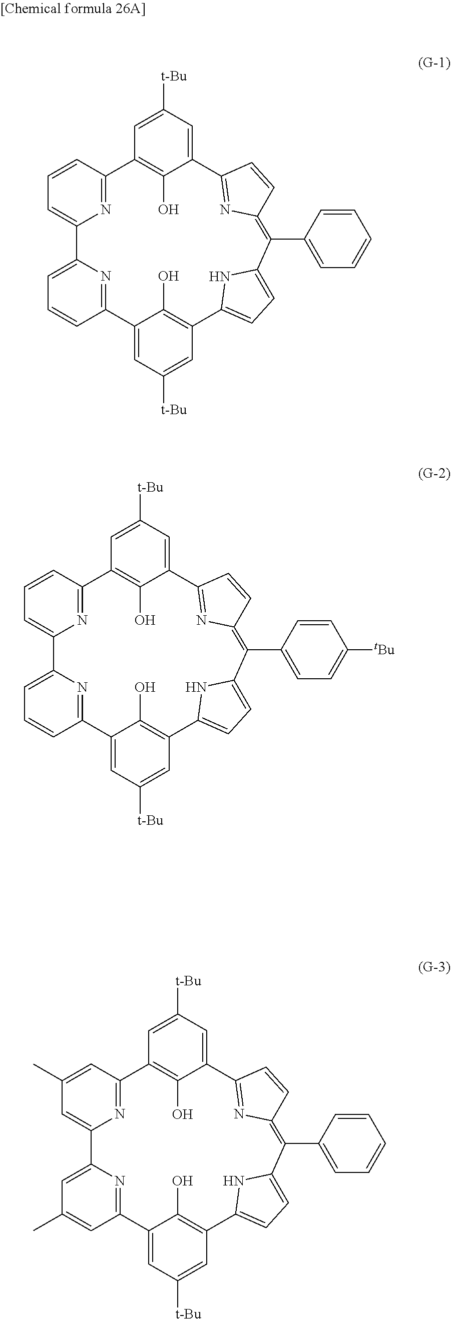 [SMILES: CC(C)(C)c1cc2c(O)c(c1)-c1cccc(n1)-c1cccc(n1)-c1cc(C(C)(C)C)cc(c1O)-c1ccc([nH]1)/C(c1ccccc1)=C1/C=CC2=N1 ; CC(C)(C)c1ccc(/C2=C3\C=CC(=N3)c3cc(C(C)(C)C)cc(c3O)-c3cccc(n3)-c3cccc(n3)-c3cc(C(C)(C)C)cc(c3O)-c3ccc2[nH]3)cc1 ; Cc1cc2nc(c1)-c1cc(C(C)(C)C)cc(c1O)C1=N/C(=C(/c3ccccc3)c3ccc([nH]3)-c3cc(C(C)(C)C)cc(c3O)-c3cc(C)cc-2n3)C=C1]

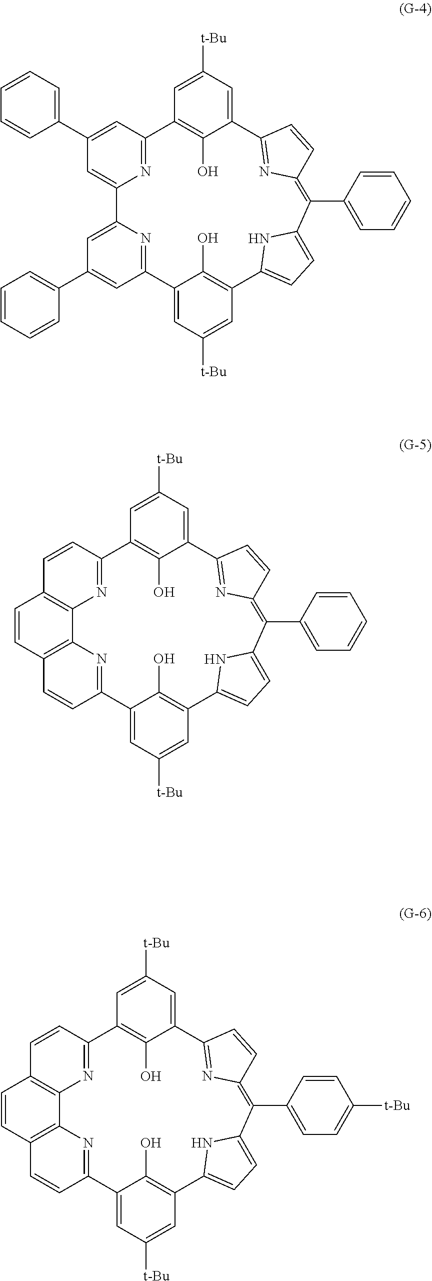 [SMILES: CC(C)(C)c1cc2c(O)c(c1)-c1cc(-c3ccccc3)cc(n1)-c1cc(-c3ccccc3)cc(n1)-c1cc(C(C)(C)C)cc(c1O)-c1ccc([nH]1)/C(c1ccccc1)=C1/C=CC2=N1 ; CC(C)(C)c1cc2c3nc(c(-c4ccccc4)c4ccc([nH]4)c4cc(C(C)(C)C)cc(c4O)c4ccc5ccc6ccc(nc6c5n4)c(c1)c2O)C=C3 ; CC(C)(C)c1ccc(-c2c3nc(c4cc(C(C)(C)C)cc(c4O)c4ccc5ccc6ccc(nc6c5n4)c4cc(C(C)(C)C)cc(c4O)c4ccc2[nH]4)C=C3)cc1]

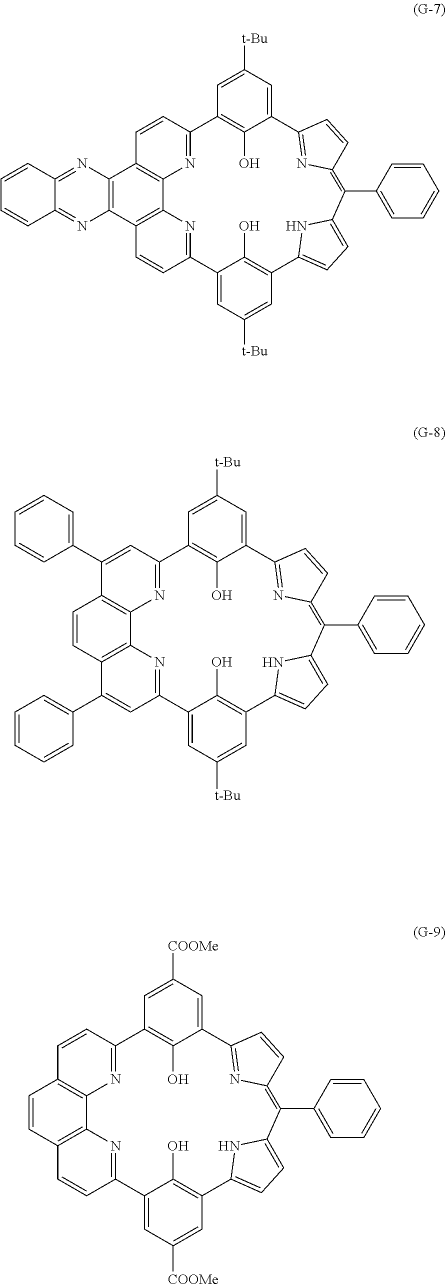 [SMILES: CC(C)(C)c1cc2c3nc(c(-c4ccccc4)c4ccc([nH]4)c4cc(C(C)(C)C)cc(c4O)c4ccc5c6nc7ccccc7nc6c6ccc(nc6c5n4)c(c1)c2O)C=C3 ; CC(C)(C)c1cc2c3nc(c(-c4ccccc4)c4ccc([nH]4)c4cc(C(C)(C)C)cc(c4O)c4cc(-c5ccccc5)c5ccc6c(-c7ccccc7)cc(nc6c5n4)c(c1)c2O)C=C3 ; COC(=O)c1cc2c3nc(c(-c4ccccc4)c4ccc([nH]4)c4cc(C(=O)OC)cc(c4O)c4ccc5ccc6ccc(nc6c5n4)c(c1)c2O)C=C3]

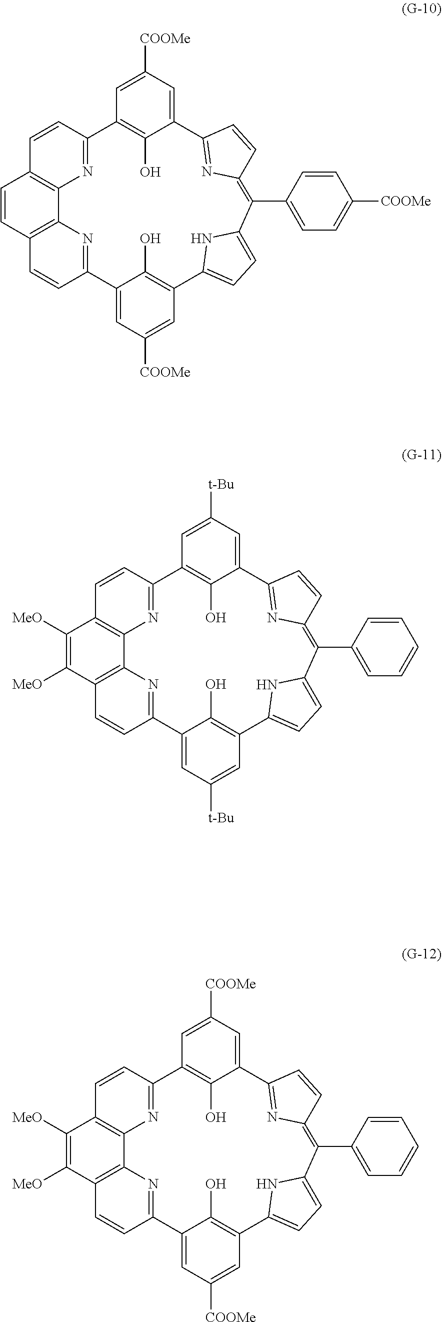 [SMILES: COC(=O)c1ccc(-c2c3nc(c4cc(C(=O)OC)cc(c4O)c4ccc5ccc6ccc(nc6c5n4)c4cc(C(=O)OC)cc(c4O)c4ccc2[nH]4)C=C3)cc1 ; COc1c(OC)c2ccc3nc2c2nc(ccc12)c1cc(C(C)(C)C)cc(c2nc(c(-c4ccccc4)c4ccc([nH]4)c4cc(C(C)(C)C)cc3c4O)C=C2)c1O ; COC(=O)c1cc2c3nc(c(-c4ccccc4)c4ccc([nH]4)c4cc(C(=O)OC)cc(c4O)c4ccc5c(OC)c(OC)c6ccc(nc6c5n4)c(c1)c2O)C=C3]

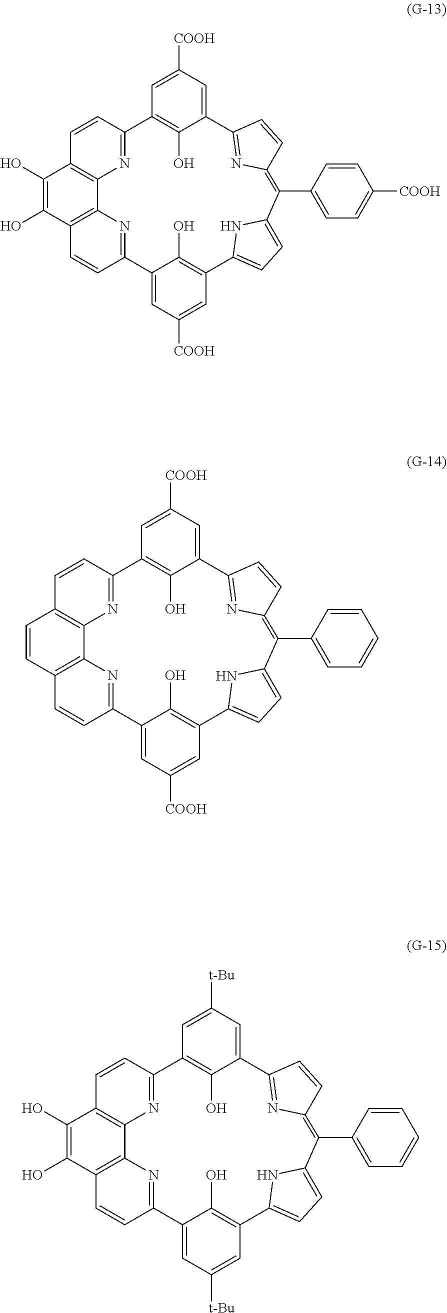 [SMILES: O=C(O)c1ccc(-c2c3nc(c4cc(C(=O)O)cc(c4O)c4ccc5c(O)c(O)c6ccc(nc6c5n4)c4cc(C(=O)O)cc(c4O)c4ccc2[nH]4)C=C3)cc1 ; O=C(O)c1cc2c3nc(c(-c4ccccc4)c4ccc([nH]4)c4cc(C(=O)O)cc(c4O)c4ccc5ccc6ccc(nc6c5n4)c(c1)c2O)C=C3 ; CC(C)(C)c1cc2c3nc(c(-c4ccccc4)c4ccc([nH]4)c4cc(C(C)(C)C)cc(c4O)c4ccc5c(O)c(O)c6ccc(nc6c5n4)c(c1)c2O)C=C3]

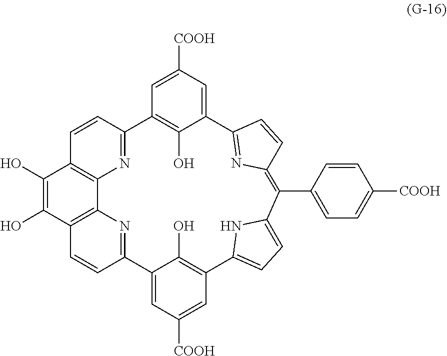 [SMILES: O=C(O)c1ccc(-c2c3nc(c4cc(C(=O)O)cc(c4O)c4ccc5c(O)c(O)c6ccc(nc6c5n4)c4cc(C(=O)O)cc(c4O)c4ccc2[nH]4)C=C3)cc1]

### (Step 3-1)

Step 3-1 is a step of performing an intramolecular cyclization reaction by, for example, causing the bipyridine derivative having two or more pyrrolyl groups optionally having a substituent, represented by the formula (3) and obtained by the operation of the deprotection step to react with a compound having an aldehyde group to obtain a precursor of a macrocyclic compound. When the bipyridine derivative represented by the formula (3) has two or more pyrrolyl groups optionally having a substituent, the bipyridine derivative undergoes a condensation reaction with a compound having an aldehyde group, and an intramolecular cyclization reaction thereby proceeds.

As a method for producing a precursor of the macrocyclic compound, as described in JP-B2-5422159 and WO-A-2019/026883, a method known as a method for causing a general pyrrole ring-containing compound and an aldehyde to undergo a condensation reaction can be applied.

In the bipyridine derivative having two or more pyrrolyl groups optionally having a substituent, represented by the formula (3), a precursor of a macrocyclic compound is preferably obtained by an intramolecular cyclization reaction between a pyrrolyl group optionally having a substituent and a compound having an aldehyde group. When the precursor of the macrocyclic compound is obtained by the intramolecular cyclization reaction, the precursor of the macrocyclic compound is represented by the following formula (12).

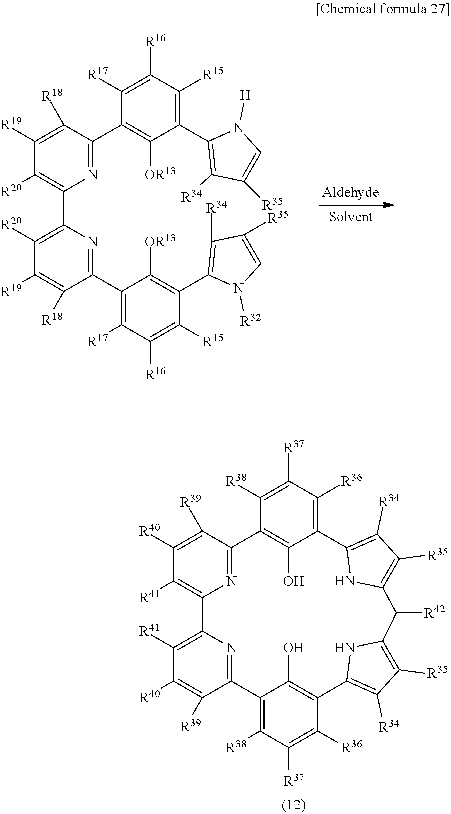

Definitions of R¹⁵ to R²⁰ in the formula (12) are the same as those in the formula (3), and definitions of R³⁴ to R⁴² are the same as those in the formula (5).

### (Step 3-2)

Step 3-2 is a step of causing the precursor of the macrocyclic compound represented by the formula (12), obtained by the operation of step 3-1 to react with an oxidizing agent or the like to perform an oxidation reaction to obtain the macrocyclic compound.

As described in JP-B2-5422159 and WO-A-2019/026883, a method known as a general method for oxidizing a dipyrromethine skeleton can be applied to the method for producing a macrocyclic compound.

A dipyrromethine skeleton is preferably oxidized, for example, by causing the precursor of the macrocyclic compound obtained by the operation of step 3-1 to react with an oxidizing agent. A case where the dipyrromethine skeleton is oxidized by an oxidizing agent is represented by the following formula (13).

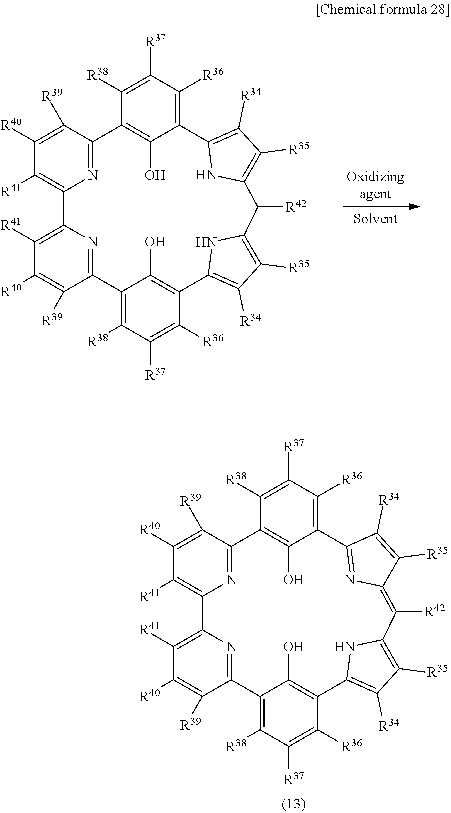

Definitions of R³⁴ to R⁴² in the formula (13) are the same as those in the formula (5).

### <<Method for Producing Metal Complex>>

A method for producing a metal complex of the present embodiment is a method for producing a metal complex containing a macrocyclic compound as a ligand, the method including causing the macrocyclic compound produced by the above production method and represented by the formula (5) as a ligand to react with a metal salt containing a metal belonging to the fourth to sixth periods of the periodic table.

A metal complex having the macrocyclic compound represented by formula (5) as a ligand will be described.

The metal complex forms a complex by interaction with a heteroatom in the macrocyclic compound. When there are two metal atoms, the metal atoms may be crosslinked and coordinated therebetween.

Among the metals belonging to the fourth to sixth periods of the periodic table, titanium, vanadium, manganese, iron, cobalt, nickel, copper, zinc, molybdenum, ruthenium, rhodium, palladium, silver, tantalum, tungsten, rhenium, osmium, iridium, platinum, and gold are preferable, titanium, vanadium, manganese, iron, cobalt, nickel, copper, zinc, molybdenum, rhodium, silver, and platinum are more preferable, and manganese, iron, cobalt, nickel, copper, and zinc are particularly preferable.

The metal complex may contain a neutral molecule or a counterion that make the metal complex electrically neutral. Examples of the neutral molecule include a molecule that is solvated to form a solvate. Examples of the neutral molecule include water, methanol, ethanol, n-propanol, isopropyl alcohol, 2-methoxyethanol, 1,1-dimethylethanol, ethylene glycol, N,N′-dimethylformamide, N,N′-dimethylacetamide, N-methyl-2-pyrrolidone, dimethylsulfoxide, acetone, chloroform, acetonitrile, benzonitrile, triethylamine, pyridine, pyrazine, diazabicyclo [2,2,2] octane, 4,4′-bipyridine, tetrahydrofuran, diethyl ether, dimethoxyethane, methyl ethyl ether, 1,4-dioxane, acetic acid, propionic acid, and 2-ethylhexanoic acid. Preferable examples of the neutral molecule include water, methanol, ethanol, isopropyl alcohol, ethylene glycol, N,N′-dimethylformamide, N,N′-dimethylacetamide, N-methyl-2-pyrrolidone, chloroform, acetonitrile, benzonitrile, triethylamine, pyridine, pyrazine, diazabicyclo [2,2,2] octane, 4,4′-bipyridine, tetrahydrofuran, dimethoxyethane, 1,4-dioxane, acetic acid, propionic acid, and 2-ethylhexanoic acid.

As for the counterion, since metals belonging to the fourth to sixth periods of the periodic table have positive charges, an anion that make the metals electrically neutral is selected. Examples of the anion include a fluoride ion, a chloride ion, a bromide ion, an iodide ion, a sulfide ion, an oxide ion, a hydroxide ion, a hydride ion, a sulfite ion, a phosphate ion, a cyanide ion, an acetate ion, a 2-ethylhexanoate ion, a carbonate ion, a sulfate ion, a nitrate ion, a perchlorate ion, a bicarbonate ion, a trifluoroacetate ion, a thiocyanate ion, a trifluoromethanesulfonate ion, an acetylacetonate, a tetrafluoroborate ion, a hexafluorophosphate ion, a tetraphenylborate ion, and a stearate ion, and a chloride ion, a bromide ion, a phosphate ion, a hexafluorophosphate ion, an acetate ion, a sulfate ion, a nitrate ion, a perchlorate ion, a trifluoromethanesulfonate ion, and a tetraphenylborate ion are preferable.

In addition, when there are a plurality of counterions, the counterions may be the same or different, and a neutral molecule and an ion may coexist.

As described in JP-B2-5422159 and WO-A-2019/026883, a method known as a method for coordinating a metal at the time of producing a general porphyrin derivative, phthalocyanine derivative, or the like can be applied to the method for producing a metal complex of the present embodiment.

The structure of the compound or the like obtained in the present invention can be confirmed by a known method such as single crystal X-ray analysis, nuclear magnetic resonance (NMR) spectroscopy, electron spin resonance (ESR) spectroscopy, mass spectrometry (MS), infrared spectroscopy (IR), or ultraviolet and visible absorption spectroscopy.

### <<Air Battery>>

The metal complex represented by the formula (6) can be used as an electrode catalyst for an air battery.

An air battery includes an electrode for air battery (cathode), an anode, and an electrolytic solution. The electrode for air battery includes a cathode current collector and a catalyst layer. The anode includes an anode current collector and an anode active material layer. The catalyst layer includes an electrode catalyst. As the electrode catalyst, the metal complex represented by the formula (6) can be used.

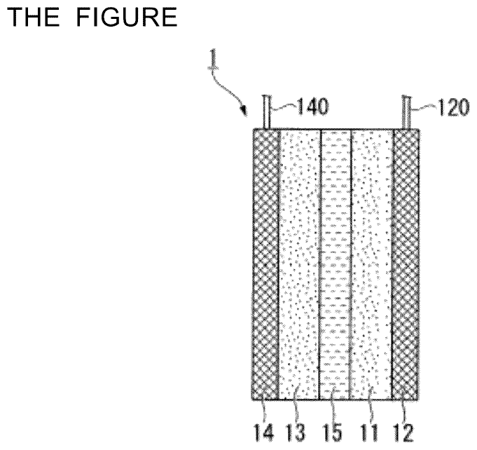

FIG. 1 is a schematic configuration diagram illustrating an embodiment of an air battery according to the present embodiment. An air battery 1 includes a catalyst layer 11, a cathode current collector 12, an anode active material layer 13, an anode current collector 14, an electrolytic solution 15, and a container (not illustrated) housing these components.

The cathode current collector 12 is disposed in contact with the catalyst layer 11, and these components constitute an electrode for air battery (cathode). The anode current collector 14 is disposed in contact with the anode active material layer 13, and these components constitute an anode. A cathode terminal (lead wire) 120 is connected to the cathode current collector 12, and an anode terminal (lead wire) 140 is connected to the anode current collector 14.

The catalyst layer 11 and the anode active material layer 13 are disposed so as to face each other, and the electrolytic solution 15 is disposed therebetween so as to be in contact therewith.

Note that the air battery is not limited to that illustrated in FIG. 1, and the configuration may be partially changed as necessary. For example, a separator may be disposed between the cathode and the anode, and an oxygen diffusion film may be disposed on a surface of the cathode current collector 12 on a side opposite to the catalyst layer 11.

### <<Electrode for Air Battery>>

The electrode for air battery is a cathode. The electrode for air battery includes a catalyst layer and a cathode current collector. The catalyst layer includes an electrode catalyst containing the metal complex represented by the formula (6). The catalyst layer preferably further includes a conductive material and a binder. As the conductive material and the binder, a conductive material and a binder described in JP-B2-5943194 or JP-B2-6830320 can be used, and the contents described in JP-B2-5943194 and JP-B2-6830320 can also be applied to the composition of the catalyst layer (contents and the like of an electrode catalyst, a conductive material, and a binder). As the cathode current collector, a cathode current collector described in JP-B2-5943194 or JP-B2-6830320 can also be used.

As a method for producing an electrode for air battery, as described in JP-B2-5943194 and JP-B2-6830320, a method for combining a catalyst layer prepared by mixing an electrode catalyst containing the metal complex represented by the formula (6), a conductive material, and a binder with a cathode current collector can be applied.

### (Anode)

The anode includes an anode active material layer containing an anode active material and an anode current collector. The anode active material preferably contains one or more selected from the group consisting of zinc, iron, aluminum, magnesium, lithium, hydrogen, and ions thereof, and more preferably contains one or more selected from the group consisting of magnesium and a magnesium ion.

When the anode active material contains one or more selected from the group consisting of magnesium (a magnesium simple substance or a magnesium compound) and a magnesium ion, the air battery is a so-called magnesium air battery.

As the anode current collector, an anode current collector described in JP-B2-5943194 or JP-B2-6830320 can be used.

As the electrolytic solution, an electrolytic solution (electrolyte) described in JP-B2-5943194 or JP-B2-6830320 can be used.

Configurations described in JP-B2-5943194 and JP-B2-6830320 can be applied to other components of the air battery (a container, a separator, an oxygen diffusion film, and the like, and the shape of the air battery and the like).

As a method for manufacturing an air battery, a method described in JP-B2-5943194 or JP-B2-6830320 can be applied.

### EXAMPLES

Hereinafter, the present invention will be described in more detail with reference to Examples, but the present invention is not limited thereto.

Hereinafter, “TMEDA” means N,N,N′,N′-tetramethylethylenediamine, “MTBE” means tert-butyl methyl ether, “THF” means tetrahydrofuran, “OAc” means an acetic acid anion, “DMSO” means dimethyl sulfoxide, “Boc” means a tert-butoxycarbonyl group, “dba” means dibenzylideneacetone, “Cy” means a cyclohexyl group, “PhCHO” means benzaldehyde, and “PhNH⁺Me₂B (C₆F₅)₄⁻” means N,N-dimethylanilinium tetrakis (pentafluorophenyl) borate.

For NMR measurement, an AV NEO 300 MHZ NMR spectrometer manufactured by BRUKER was used.

### Example 1

### Synthesis of Metal Complex (B-8)

A metal complex (B-8) was synthesized according to the following reaction formula.

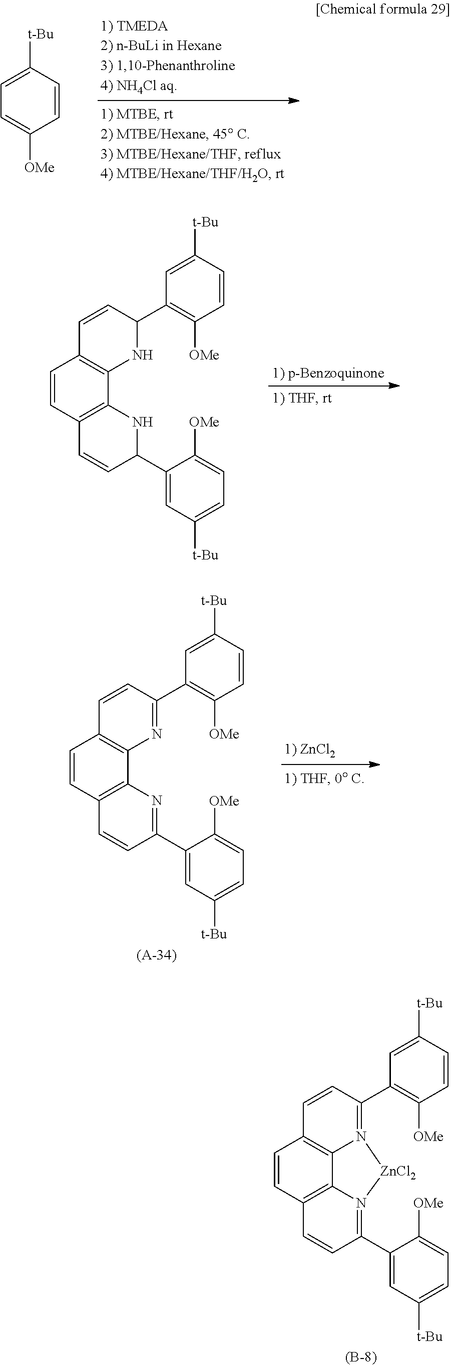

The inside of a reaction vessel was brought into a nitrogen gas atmosphere. Thereafter, 135 mL of MTBE, 63.80 g (388 mmol) of 4-tert-butyl anisole, and 38.69 g (333 mmol) of TMEDA were added dropwise into the reaction vessel, and the mixture was cooled to 0° C. To the mixture, 212.07 mL (1.6 mol/L, 333 mmol as n-butyllithium) of a hexane solution of n-butyllithium was added dropwise. The temperature was raised to 45° C. Thereafter, the mixture was stirred for 1.5 hours to obtain a lithiation reaction liquid. The inside of another reaction vessel was brought into a nitrogen gas atmosphere. Thereafter, 10.00 g (55.5 mmol) of anhydrous 1,10-phenanthroline was suspended in 113 mL of THE at room temperature. This suspension was added dropwise to the lithiation reaction liquid. Thereafter, the temperature was raised to 65° C., and the mixture was stirred for two hours while being refluxed to obtain an arylation reaction liquid. To the arylation reaction liquid cooled to room temperature, 100.00 g of a 20% by mass ammonium chloride aqueous solution was added dropwise.

The mixture was stirred for 30 minutes to be washed, and the aqueous phase was removed. Thereafter, the organic phase was concentrated under reduced pressure. The inside of another reaction vessel was brought into a nitrogen gas atmosphere. Thereafter, 12.00 g (111 mmol) of p-benzoquinone was dissolved in 113 mL of THF at room temperature. This solution was added dropwise to the concentrated organic phase, and the mixture was stirred at room temperature for 30 minutes to obtain an oxidation reaction liquid containing a compound (A-34).

The inside of another reaction vessel was brought into a nitrogen gas atmosphere. Thereafter, 11.35 g (83.2 mmol) of zinc chloride was suspended in 113 mL of THF at room temperature. This suspension was added dropwise to the oxidation reaction liquid at room temperature. The obtained suspension was cooled to 0° C. and then stirred for four hours. Thereafter, the suspension was filtered at 0° C., washed with THF, and then dried under reduced pressure to obtain the metal complex (B-8) at a yield of 55%. Identification data of the obtained metal complex (B-8) is described below. The metal complex (B-8) corresponds to the metal complex 1 in the present invention.

¹H-NMR (300 MHz, CDCl₃): δ (ppm)=1.37 (s, 18H), 3.76 (s, 6H), 6.98 (d, J=9.0 Hz, 2H), 7.52 (dd, J=9.0 Hz, 2.4 Hz, 2H), 7.87 (d, J=2.4 Hz, 2H), 8.02 (d, J=8.4H z, 2H), 8.02 (s, 2H), 8.50 (d, J=8.4 Hz, 2H)

### Synthesis of Metal Complex (B-24)

A metal complex (B-24) was synthesized according to the following reaction formula.

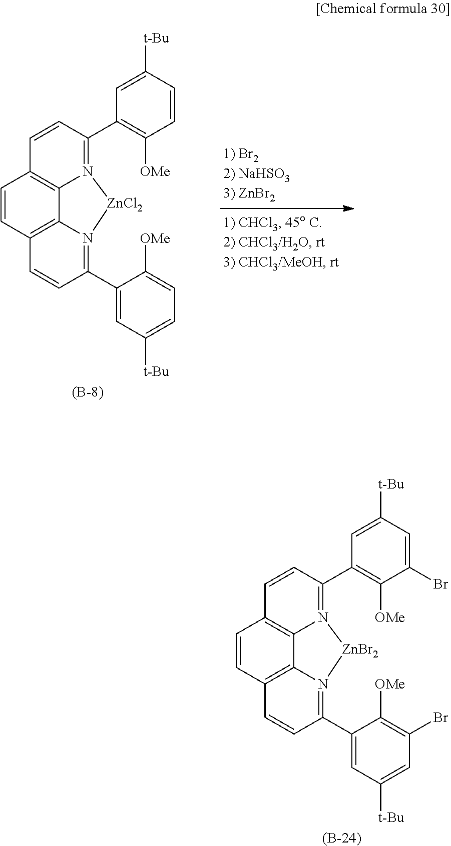

The inside of a reaction vessel was brought into a nitrogen gas atmosphere. Thereafter, 8.00 g (12.48 mmol) of the metal complex (B-8) was added to and dissolved in 108 mL of chloroform at room temperature. To the solution, 15.96 g (99.85 mol) of bromine was added dropwise with stirring, and the temperature was raised to 45° C. Thereafter, the mixture was stirred for six hours to obtain a bromination reaction liquid. The inside of another reaction vessel was brought into a nitrogen gas atmosphere. Thereafter, 10.39 g (99.85 mmol) of sodium thiosulfate was dissolved in 160 mL of water at room temperature. This aqueous solution was added dropwise to the bromination reaction liquid cooled to 0° C. The mixture was stirred for one hour to be washed. Thereafter, the aqueous phase was removed. The inside of another reaction vessel was brought into a nitrogen gas atmosphere. Thereafter, 2.81 g (12.48 mmol) of zinc bromide was dissolved in 151 mL of methanol at room temperature. This solution was added to the organic phase after washing. Thereafter, the temperature was raised to 75° C., and the mixture was concentrated. To the concentrate, 202 mL of methanol was added, and the mixture was stirred for one hour while being refluxed at 75° C. The mixture was cooled to 0° C., stirred for one hour, then filtered, washed with methanol, and then dried under reduced pressure to obtain the metal complex (B-24) at a yield of 88%. Identification data of the obtained metal complex (B-24) is described below. The metal complex (B-24) corresponds to the metal complex 2 (halogenated product of the metal complex 1) in the present invention.

¹H-NMR (300 MHz, CDCl₃): δ (ppm)=1.36 (s, 18H), 3.65 (s, 6H), 7.63 (d, J=2.4 Hz, 2H), 7.87 (s, 2H), 7.93 (d, J=2.4 Hz, 2H), 8.17 (d, J=8.1 Hz, 2H), 8.31 (d, J=8.1 Hz, 2H)

### Synthesis of Compound (C-17)

A compound (C-17) was synthesized according to the following reaction formula.

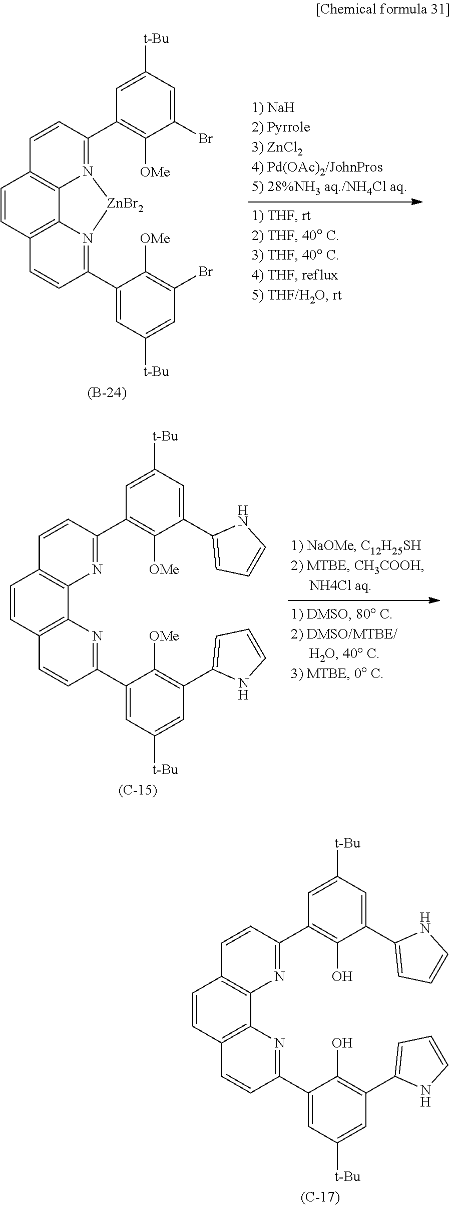

The inside of a reaction vessel was brought into a nitrogen gas atmosphere. Thereafter, 106 mL of THE was added to 4.39 g (110 mmol) of sodium hydride to suspend the mixture. The temperature of the suspension was raised to 40° C., 32.67 g (487 mmol) of pyrrole was added dropwise to the suspension over 20 minutes, and the mixture was stirred for 30 minutes to obtain a reaction liquid. The inside of another reaction vessel was brought into a nitrogen gas atmosphere, and 19.96 g (146 mmol) of zinc chloride was suspended in 137 mL of THE at room temperature. The suspension was added dropwise to the reaction liquid. The mixture was stirred for 30 minutes and then cooled to room temperature. To the mixture, 32.50 g (36.6 mmol) of the metal complex (B-24) was added. The inside of another reaction vessel was brought into a nitrogen gas atmosphere. Thereafter, 0.082 g (0.37 mmol) of palladium acetate and 0.219 g (0.73 mmol) of 2-(di-tert-butylphosphino) biphenyl were dissolved in 6.5 mL of THF at room temperature to obtain a catalyst solution. The catalyst solution was added dropwise to the reaction liquid. Thereafter, the temperature was raised to 75° C., and the mixture was stirred for six hours while being refluxed, and then cooled to room temperature.

The inside of another reaction vessel was brought into a nitrogen gas atmosphere. Thereafter, 86.23 g of ammonium chloride and 178.15 g of an ammonia aqueous solution (28%, 2930 mmol) were dissolved in 217 mL of water at room temperature. The aqueous solution was added dropwise to the reaction liquid. The mixture was stirred at room temperature for 30 minutes to be washed, and the aqueous phase was removed. To the obtained organic phase, 216 g of a 24.8% by mass ammonium chloride aqueous solution was added dropwise, the mixture was stirred for 15 minutes to be washed, and the aqueous phase was removed.

To the obtained organic phase, 79 mL of DMSO was added, the temperature was raised to 82° C., and THF was removed by concentration under reduced pressure. To the resulting liquid, 6.18 g (30.5 mmol) of 1-dodecanethiol and 7.06 g (28%, 36.6 mmol as sodium methoxide) of a methanol solution of sodium methoxide were added dropwise, and the mixture was stirred at 82° C. for 6.5 hours. The reaction liquid was cooled to 40° C., and 58.6 mL of MTBE was added to the reaction liquid. The inside of another reaction vessel was brought into a nitrogen gas atmosphere. Thereafter, 23.50 g of ammonium chloride and 2.93 g (48.8 mmol) of acetic acid were dissolved in 86.7 mL of water at room temperature. The aqueous solution was added dropwise to the reaction liquid. The mixture was stirred at 40° C. for 30 minutes to be washed, and the aqueous phase was removed. The obtained organic phase was cooled to 0° C., stirred for two hours, and then filtered. The obtained crystals were washed with MTBE and methanol in this order and dried under reduced pressure to obtain the compound (C-17) at a yield of 76%. Identification data of the obtained metal complex (C-17) is described below. Note that a compound (C-15) and the compound (C-17) correspond to the bipyridine derivative in the present invention. The compound (C-17) is a deprotected product.

¹H-NMR (300 MHz, CDCl₃): δ (ppm)=1.40 (s, 18H), 6.25 (m, 2H), 6.44 (m, 2H), 6.74 (m, 2H), 7.84 (s, 2H), 7.89 (s, 2H), 7.92 (s, 2H), 8.35 (d, J=8.4 Hz, 2H), 8.46 (d, J=8.4 Hz, 2H), 10.61 (s, 2H), 15.88 (s, 2H)

### Synthesis of Compound (G-5)

A compound (G-5) was synthesized according to the following reaction formula by a method described in WO-A-2019/026883. Note that the compound (G-5) corresponds to the macrocyclic compound in the present invention.

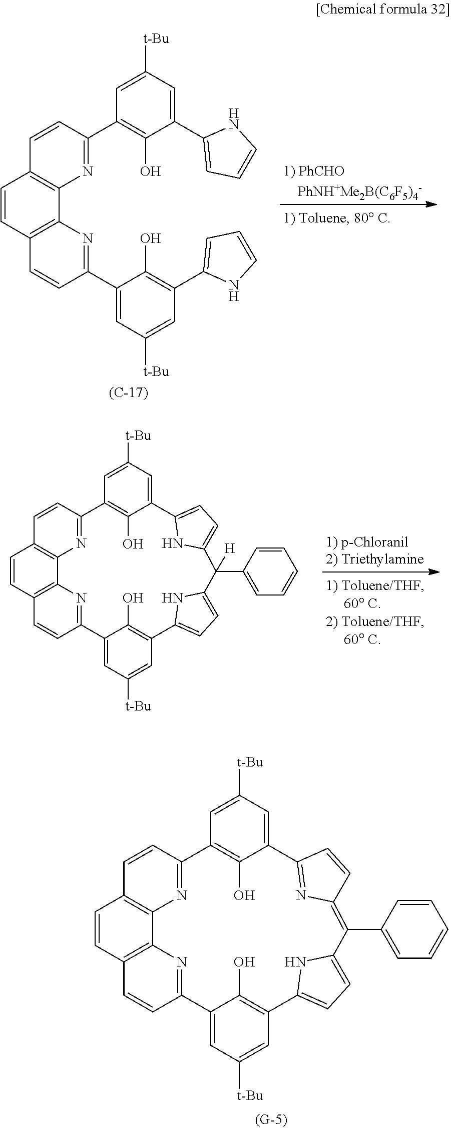

### Synthesis of Metal Complex Having Macrocyclic Compound (G-5) as Ligand

A metal complex having a macrocyclic compound (G-5) as a ligand was synthesized according to the following reaction formula by a method described in WO-A-2019/026883.

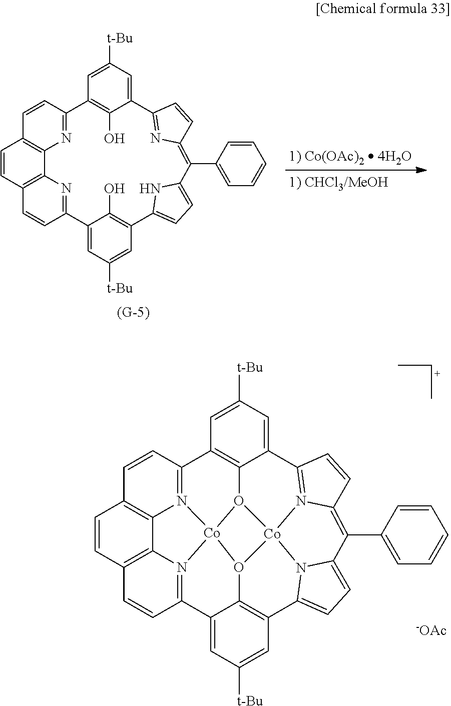

### Comparative Example 1

### Synthesis of Compound (A-34)

A compound (A-34) was synthesized according to the following reaction formula.

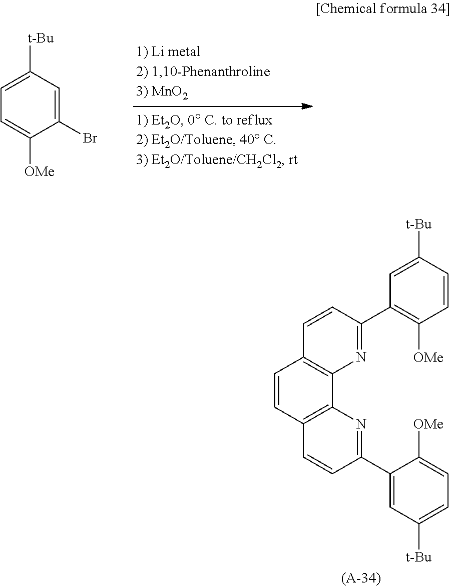 [SMILES: COc1ccc(C(C)(C)C)cc1Br ; COc1ccc(C(C)(C)C)cc1-c1ccc2ccc3ccc(-c4cc(C(C)(C)C)ccc4OC)nc3c2n1 ; "(A-34)"]

The inside of a reaction vessel was brought into a nitrogen gas atmosphere. Thereafter, 1.00 g (143 mmol) of metallic lithium was suspended in 10 mL of anhydrous diethyl ether, and the suspension was cooled to 0° C. To the suspension, a liquid prepared by dissolving 15.50 g (63.8 mmol) of 2-bromo-4-(1,1-dimethylethyl)-1-methoxybenzene (synthesized according to the description of Tetrahedron; 1999, 55, 8377.) in 10 mL of anhydrous diethyl ether was added dropwise, the temperature was raised, and the mixture was stirred for three hours while being refluxed. The mixture was cooled to room temperature to obtain a lithiation reaction liquid. The inside of another reaction vessel was brought into a nitrogen gas atmosphere. Thereafter, 1.44 g (7.97 mmol) of anhydrous 1,10-phenanthroline was suspended in 15 mL of anhydrous toluene at room temperature. The lithiation reaction liquid was added dropwise to the suspension at room temperature, the temperature was raised to 40° C., and the mixture was stirred for 48 hours while being refluxed. To the mixture, 150 mL of water was added dropwise while the mixture was cooled to −20° C. The temperature was returned to room temperature. Thereafter, dichloromethane was added to the mixture, extraction was performed, and the aqueous phase was removed. To the obtained organic phase, 5.00 g (57.0 mmol) of manganese dioxide was added, and the mixture was stirred at room temperature for eight hours. The obtained suspension was filtered through a celite-filled funnel.

Anhydrous sodium sulfate was added to the filtrate, and the mixture was allowed to stand and then filtered. The obtained organic phase was concentrated. The residue was purified with a silica gel column using a mixed liquid of ethyl acetate and petroleum ether as a developing solvent to obtain the compound (A-34) at a yield of 64%. Identification data of the obtained compound (A-34) is described below.

¹H-NMR (300 MHz, CDCl₃): δ (ppm)=1.38 (s, 18H), 3.83 (s, 6H), 6.97 (d, J=8.7 Hz, 2H), 7.42 (dd, J=8.7 Hz, 2.7 Hz, 2H), 7.80 (s, 2H), 8.05 (d, J=2.7 Hz, 2H), 8.08 (d, J=8.4 Hz, 2H), 8.22 (d, J=8.4 Hz, 2H)

### Synthesis of Compound (C-12)

Compound (C-12) was synthesized according to the following reaction formula.

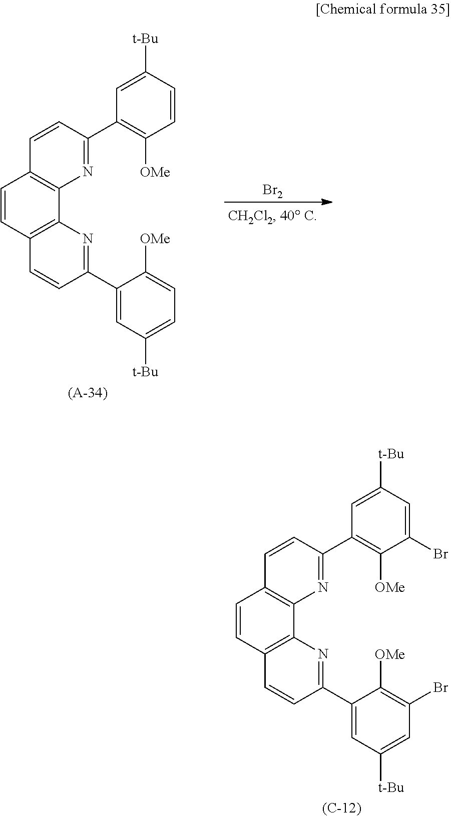 [SMILES: COc1ccc(C(C)(C)C)cc1-c1ccc2ccc3ccc(-c4cc(C(C)(C)C)ccc4OC)nc3c2n1 ; "Br2" ; "(A-34)" ; COc1c(Br)cc(C(C)(C)C)cc1-c1ccc2ccc3ccc(-c4cc(C(C)(C)C)cc(Br)c4OC)nc3c2n1 ; "(C-12)"]

The inside of a reaction vessel was brought into a nitrogen gas atmosphere. Thereafter, 260.00 g (0.515 mol) of the compound (A-34) was dissolved in 15 L of dichloromethane at room temperature. To the solution, 658.66 g (4.12 mol) of bromine was added dropwise with stirring. The temperature was raised to 40° C., and the mixture was stirred for 48 hours. To the mixture, 658.66 g (4.12 mol) of bromine was additionally added dropwise while the temperature was kept at 40° C., and the mixture was further stirred for 48 hours.

The mixture was cooled to 0° C., and 500 mL of a sodium thiosulfate aqueous solution having a concentration of 10% was added to the mixture.

The aqueous phase was removed. A sodium thiosulfate aqueous solution was added to the organic phase, and the mixture was stirred. Thereafter, the aqueous phase was removed. A sodium bicarbonate aqueous solution was added to the organic phase, and the mixture was stirred. Thereafter, the aqueous phase was removed. A saline solution was added to the organic phase, and the mixture was stirred. Thereafter, the aqueous phase was removed. Anhydrous sodium sulfate was added to the organic phase, and the mixture was allowed to stand. Thereafter, the mixture was filtered, and the obtained organic phase was concentrated. The residue was purified with a silica gel column using a mixed liquid of hexane and ethyl acetate as a developing solvent to obtain the compound (C-12) at a yield of 52%. Identification data of the obtained metal complex (C-12) is described below.

¹H-NMR (300 MHz, CDCl₃): δ (ppm)=1.36 (s, 18H), 3.65 (s, 6H), 7.63 (d, J=2.4 Hz, 2H), 7.87 (s, 2H), 7.93 (d, J=2.4 Hz, 2H), 8.17 (d, J=8.1 Hz, 2H), 8.31 (d, J=8.1 Hz, 2H)

### Synthesis of Compound (C-16)

Compound (C-16) was synthesized according to the following reaction formula.

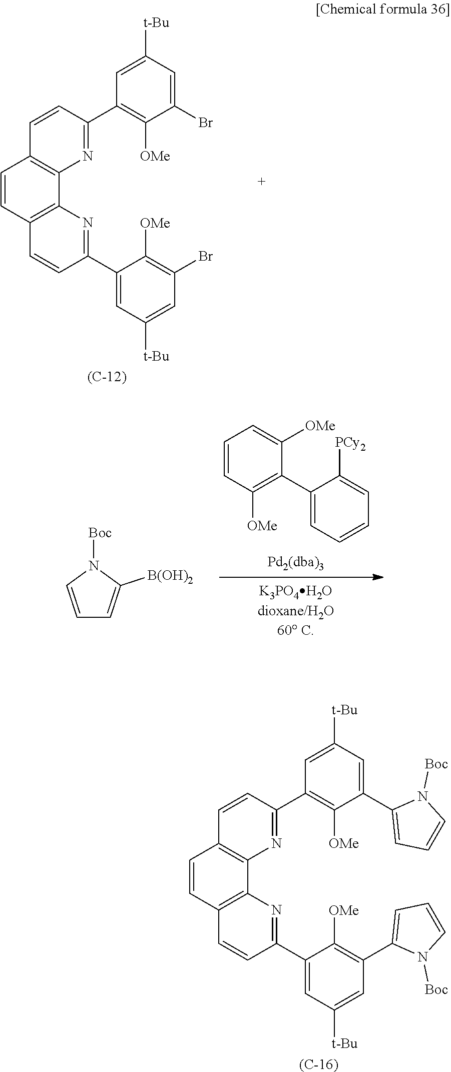

The inside of a reaction vessel was brought into a nitrogen gas atmosphere. Thereafter, 150.00 g (0.226 mol) of the compound (C-12), 119.45 g (0.566 mmol) of 1-N-Boc-pyrrole-2-boronic acid, 5.18 g (5.66 mmol) of tris (benzylideneacetone) dipalladium, 9.30 g (22.6 mmol) of 2-dicyclohexylphosphino-2′,6′-dimethoxybiphenyl, and 210.00 g (0.989 mol) of potassium phosphate were added to a mixed solvent of 7500 mL of dioxane and 750 ml of water to be dissolved in the mixed solvent. The temperature was raised to 60° C., and the mixture was stirred for six hours. The reaction liquid was cooled to room temperature and filtered through a celite-filled funnel. Distilled water and chloroform were added to the filtrate, the mixture was liquid-separated, and the aqueous phase was removed. Anhydrous sodium sulfate was added to the obtained organic phase, and the mixture was allowed to stand and then filtered. The obtained organic phase was concentrated. The residue was purified with a silica gel column to obtain the compound (C-16) at a yield of 63%. Identification data of the obtained metal complex (C-16) is described below.

¹H-NMR (300 MHz, CDCl₃): δ (ppm)=1.34 (s, 18H), 1.37 (s, 18H), 3.30 (s, 6H), 6.2 1 (m, 2H), 6.27 (m, 2H), 7.37 (m, 2H), 7.41 (s, 2H), 7.82 (s, 2H), 8.00 (s, 2H), 8.19 (d, J=8.6 Hz, 2H), 8.27 (d, J=8.6 Hz, 2H)

### Synthesis of Compound (C-17)

A compound (C-17) was synthesized according to the following reaction formula.

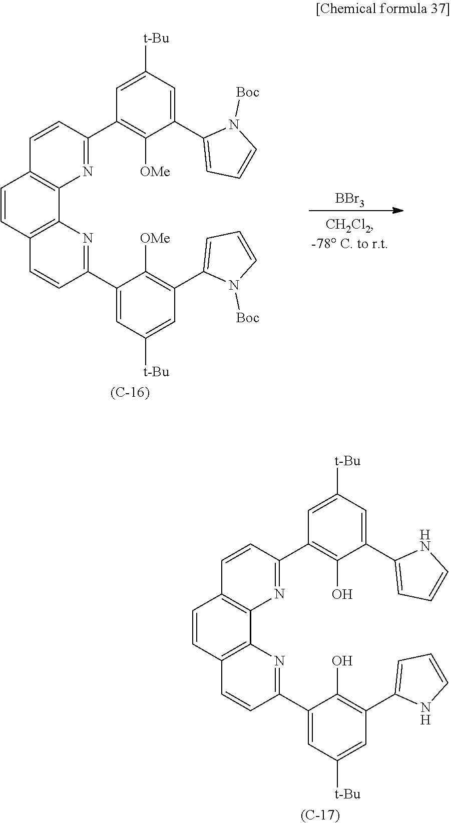

The inside of a reaction vessel was brought into a nitrogen gas atmosphere. Thereafter, 74.0 g (88.6 mmol) of the compound (C-16) was dissolved in 740 mL of anhydrous dichloromethane. To the obtained dichloromethane solution, 740 mL of a 1.0 M dichloromethane solution of boron tribromide (740 mmol as boron tribromide) was added dropwise while the dichloromethane solution was cooled to −78° C. After the dropwise addition, the mixture was stirred for 30 minutes. Thereafter, the temperature was gradually raised over two hours to room temperature. The reaction liquid was cooled to −20° C., and 1600 mL of water was added to the reaction liquid. The temperature of the mixture was raised to room temperature. A saturated sodium bicarbonate aqueous solution was added to the mixture, and the mixture was stirred. Thereafter, the aqueous phase was removed, hydrochloric acid was added to the organic phase, and the mixture was stirred. Thereafter, the aqueous phase was removed. Anhydrous sodium sulfate was added to the obtained organic phase, and the mixture was allowed to stand and then filtered. The obtained organic phase was concentrated. The residue was purified with a silica gel column using a mixed liquid of chloroform and hexane as a developing solvent to obtain the compound (C-17) at a yield of 46%. Identification data of the obtained metal complex (C-17) is described below.

¹H-NMR (300 MHz, CDCl₃): δ (ppm)=1.40 (s, 18H), 6.25 (m, 2H), 6.44 (m, 2H), 6.74 (m, 2H), 7.84 (s, 2H), 7.89 (s, 2H), 7.92 (s, 2H), 8.35 (d, J=8.4 Hz, 2H), 8.4 6 (d, J=8.4 Hz, 2H), 10.61 (s, 2H), 15.88 (s, 2H)

Table 1 below presents purification methods, yields, and overall yields in Example in which purification was performed by crystallization filtration via a metal complex as an intermediate and Comparative Example in which purification was performed by column chromatography without via a metal complex. Note that an organic phase containing the compound (C-15) was used as a solution in a subsequent step. Note that the yield was determined by measuring the mass of a target product, dividing the mass by a theoretical yield (mass in a case where the yield is 100%), and multiplying the calculated value by 100%.

TABLE 1

| | | | | | Overall |
|---|---|---|---|---|---|
| | Yield (Taking-out method) | | | | yield |
| Example 1 | Metal complex (B-8) | Metal complex (B-24) | Compound (C-15) | Compound (C-17) | 37% |
| | 55% (Crystallization | 88% (Crystallization | — | 76% (Crystallization | |
| | filtration) | filtration) | | filtration) | |
| Comparative | Compound (A-34) | Compound (C-12) | Compound (C-16) | Compound (C-17) | 10% |
| Example 1 | 64% (Column) | 52% (Column) | 63% (Column) | 46% (Column) | |

Note that the compound (C-17) was isolated by crystallization filtration in Example 1 and by column in Comparative Example 1. In Example 1, deprotection was performed using dodecanethiol and sodium methoxide, and a reaction yield was high. Therefore, it is considered that isolation by crystallization filtration was possible. Meanwhile, in Comparative Example 1, deprotection was performed using boron tribromide, and a reaction yield was low (that is, a ratio of impurities was high). Therefore, it is considered that isolation by crystallization filtration was impossible. Note that, when a yield up to the compound (C-15) in Example 1 is compared with a yield up to the compound (C-16) in Comparative Example 1, the yield in Example 1 is 48%, and the yield in Comparative Example 1 is 21%. Even when deprotection is performed by the same method, a yield of the compound (C-17) in Example 1 is higher than that in Comparative Example 1.

From the above, it has been found that by using the production method of the present invention, purification by crystallization filtration is possible, and a bipyridine derivative can be produced at a higher yield than that in a case where a production method without via a metal complex as an intermediate is used.

### DESCRIPTION OF REFERENCE SIGNS

1 Air battery
11 Catalyst layer
12 Cathode current collector
13 Anode active material layer
14 Anode current collector
120 Cathode terminal
140 Anode terminal
15 Electrolyte

1. A method for producing a bipyridine derivative, comprising:
a first step of obtaining a metal complex 1 represented by the following formula (2) from a compound represented by the following formula (1); and
a second step of obtaining a bipyridine derivative represented by the following formula (3) from the metal complex 1, wherein
the second step includes one or both step of a halogenation reaction and a pyrrole group introducing reaction on the metal complex 1 to obtain a metal complex 2, and a demetalation step of the metal complex 2, and
the number of halogen atoms contained in the bipyridine derivative is larger than the number of halogen atoms contained in the compound, or the number of pyrrolyl groups optionally having a substituent, contained in the bipyridine derivative is larger than the number of pyrrolyl groups optionally having a substituent, contained in the compound: 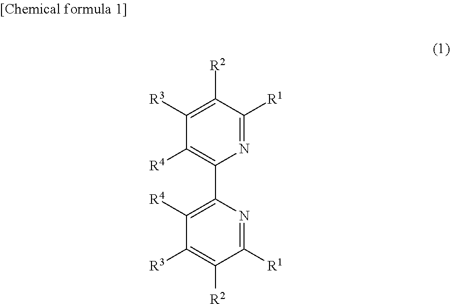
(in the formula (1), R¹ to R⁴ each independently represent a hydrogen atom or a substituent, R¹ to R⁴ may be the same or different, two R¹s may be the same or different, two R²s may be the same or different, two R³s may be the same or different, two R⁴s may be the same or different, any two substituents of R¹ to R⁴ may be bonded to each other to form a ring, and each of R¹ to R⁴ may contain a halogen atom or a pyrrolyl group optionally having a substituent), 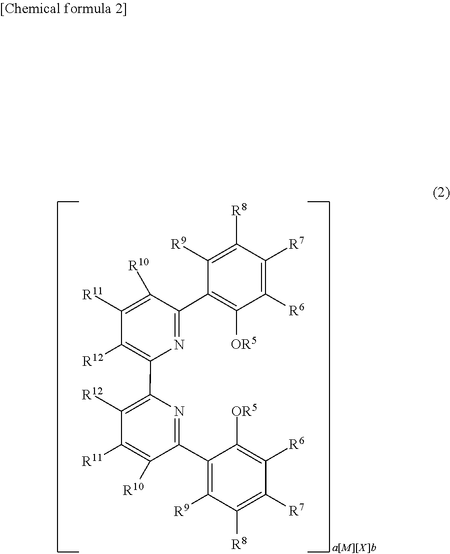
(in the formula (2), R⁵ to R¹² each independently represent a hydrogen atom or a substituent, R⁵ to R¹² may be the same or different, two R⁵s may be the same or different, two R⁶s may be the same or different, two R⁷s may be the same or different, two R⁸s may be the same or different, two R⁹s may be the same or different, two R¹⁰s may be the same or different, two R¹¹s may be the same or different, two R¹²s may be the same or different, at least one of six Rs consisting of two R⁶s, two R⁷s, and two R⁸s represents a substituent, at least one of two R⁹s represents a hydrogen atom, any two substituents of R⁵ to R¹² may be bonded to each other to form a ring, R⁶ to R¹² may each contain a halogen atom or a pyrrolyl group optionally having a substituent, M represents any metal belonging to Groups 4 to 12 in fourth period of periodic table, X represents an anion species, a represents an integer of 1 to 3, and b represents 0 or more), and 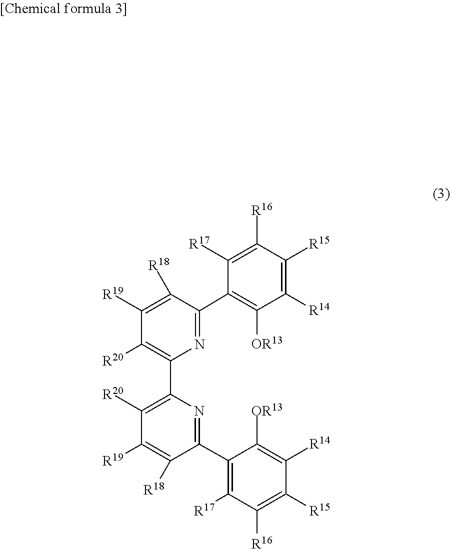
(in the formula (3), R¹³ to R²⁰ each independently represent a hydrogen atom or a substituent, R¹³ to R²⁰ may be the same or different, two R¹³s may be the same or different, two R¹⁴s may be the same or different, two R¹⁵s may be the same or different, two R¹⁶s may be the same or different, two R¹⁷s may be the same or different, two R¹⁸s may be the same or different, two R¹⁹s may be the same or different, two R²⁰s may be the same or different, at least one of six Rs consisting of two R¹⁴s, two R¹⁵s, and two R¹⁶s represents a substituent, at least one of two R¹⁷s represents a hydrogen atom, any two substituents of R¹³ to R²⁰ may be bonded to each other to form a ring, each of R¹⁷ to R²⁰ may contain a halogen atom or a pyrrolyl group optionally having a substituent, and at least one of R¹⁴ to R¹⁶ contains a halogen atom or a pyrrolyl group optionally having a substituent).

2. The method for producing a bipyridine derivative according to claim 1, wherein the demetalation step is performed by using an amine represented by the following formula (4): 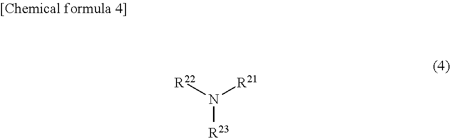
(in the formula (4), R²¹ to R²³ each independently represent a hydrogen atom or a substituent).

3. The method for producing a bipyridine derivative according to claim 1, wherein the first step includes a step for reacting the compound and a metal salt containing a metal represented by the M and an anion species represented by the X.

4. The method for producing a bipyridine derivative according to claim 1, wherein the second step includes a deprotection step after the demetalation step.

5. The method for producing a bipyridine derivative according to claim 1, comprising a step of isolating the metal complex 1, the metal complex 2, or the bipyridine derivative by crystallization.

6. A method for producing a macrocyclic compound, comprising ring-closing reaction of the bipyridine derivative having two or more pyrrolyl groups optionally having a substituent, which has been produced by the method for producing a bipyridine derivative according to claim 1 to obtain a macrocyclic compound represented by the following formula (5): 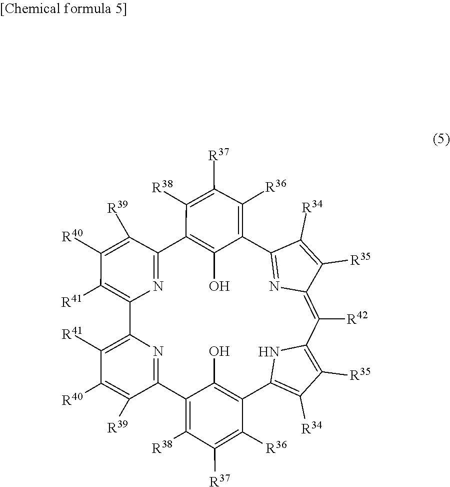
(in the formula (5), R³⁴ to R⁴² each independently represent a hydrogen atom or a substituent, R³⁴ to R⁴² may be the same or different, two R³⁴s may be the same or different, two R³⁵s may be the same or different, two R³⁶s may be the same or different, two R³⁷s may be the same or different, two R³⁸s may be the same or different, two R³⁹s may be the same or different, two R⁴⁰s may be the same or different, two R⁴¹s may be the same or different, and any two substituents of R³⁴ to R⁴² may be bonded to each other to form a ring).

7. A method for producing a metal complex containing a macrocyclic compound as a ligand, the method comprising the reaction of the macrocyclic compound produced by the method for producing a macrocyclic compound according to claim 6 as a ligand with a metal salt containing a metal belonging to fourth to sixth periods of periodic table.

8. A metal complex represented by the following formula (6): 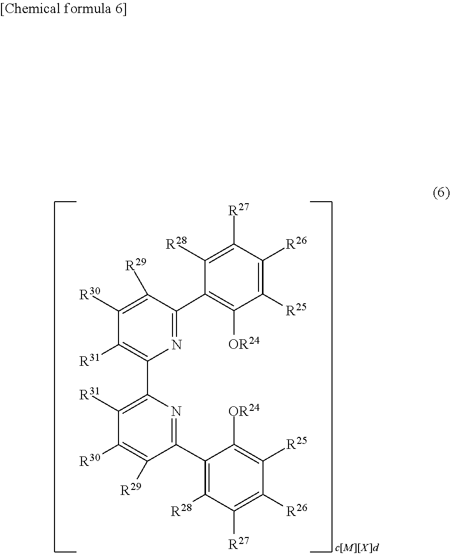
(in the formula (6), R²⁴ represents a substituent, R²⁵ to R³¹ each independently represent a hydrogen atom or a substituent, R²⁴ to R³¹ may be the same or different, two R²⁴s may be the same or different, two R²⁵s may be the same or different, two R²⁶s may be the same or different, two R²⁷s may be the same or different, two R²⁸s may be the same or different, two R³⁰s may be the same or different, two R³¹s may be the same or different, at least one of six Rs consisting of two R²⁵s, two R²⁶s, and two R²⁷s represents a substituent, at least one of two R²⁸s represents a hydrogen atom, any two substituents of R²⁴ to R³¹ may be bonded to each other to form a ring, M represents any metal belonging to Groups 4 to 12 in fourth period of periodic table, X represents an anion species, c represents an integer of 1 to 3, and d represents 0 or more).

9. An electrode for air battery, comprising a catalyst layer including an electrode catalyst containing the metal complex according to claim 8, a conductive material, and a binder.

10. An air battery comprising: the electrode for air battery according to claim 9; and an anode, wherein the anode contains an anode active material, and the anode active material contains one or more selected from the group consisting of zinc, iron, aluminum, magnesium, lithium, hydrogen, and ions thereof.

11. The air battery according to claim 10, wherein the anode active material contains one or more selected from the group consisting of magnesium and magnesium ions.
